(19) United States
(12) Patent Application Publication
(10) Pub. No.: US 2026/0157674 A1
(43) Pub. Date: Jun. 11, 2026

(54) Equipment for increasing of leads of ECG devices

(71) Applicant: Vladimir Kranz, Melville, NY (US)

(72) Inventor: Vladimir Kranz, Melville, NY (US)

(21) Appl. No.: 18/652,872

(22) PCT Filed: Mar. 15, 2021
(86) PCT No.: PCT/CZ2021/000010

§ 371 Date: May 2, 2024

(30) Foreign Application Priority Data
Mar. 13, 2020 (CZ) PV2020-141

(51) Int. Cl. A61B 5/256; A61B 5/00; A61B 5/304; A61B 5/339

(52) U.S. Cl. CPC A61B 5/256; A61B 5/304; A61B 5/339; A61B 5/681

(57) ABSTRACT

This invention relates to a monitor of the cardiac signals with the possibility to increase the number of the measured leads and to go over from the basic short-term measuring to the long-term one. The monitor is formed by the two-channel or multi-channel monitor, and from the basic sensing of the cardiac signals with the primary electrodes, for example by one lead, it is possible to go over to the multi-lead sensing with further secondary electrodes, which are located on the sensing unit. The electrodes are switched over individually or in sets with the connecting field. The cooperating unit evaluates and displays the processed signals.

### FIELD OF THE INVENTION

This equipment relates to monitoring of cardiac activity.

### BACKGROUND OF THE INVENTION

The small and the handheld equipment for short-term operative monitoring of the cardiac activity by the monitored person by attaching the fingers to the monitoring equipment, or by attaching the monitoring equipment to the chest according to the prior art are designed only for sensing and displaying of the one ECG lead and they do not allow sensing of any of the V1 to V6 chest leads for finding of the possible elevation of the ST segment for detection of an acute myocardial infarction (AIM) and they do not have automatic evaluation of AIM for alarm activation in such case to warn the monitored person. This means that for the general population there is not any available handheld device, which persons would carry with them, so that with such device they could confirm or exclude an AIM, especially in case such symptoms exist, and transmit the results of such test to the rescue service team to speed up some life-saving action.

But the ischemic heart disease, which causes AIM, is the worldwide most frequent cause of death, and it is currently responsible for almost 1.8 million deaths a year, thus, for 20% of all deaths in Europe. In European countries, its incidence ranged from 43 to 144 per 100,000 inhabitants per year. Mortality in patients with AIM is affected by the time required to diagnose AIM, and the time it takes to begin treatment. A handheld device that would allow simple monitoring of cardiac activity, such as for example by a single lead, but which would switch to monitoring of up to 21 leads, if necessary, to exclude or confirm an infarct, is not available on the market. The ECG devices measure 12 leads and for confirming of a suspected infarct, the electrodes are reshuffled in a complex way. The ECG for a simple switching from 12 to 21 leads is not available on the market. The Holters or the monitors available at a hospital bed monitor up to 7 leads. There is not any possibility to measure up to 21 leads by a simple switching over, to exclude an infarct. But there is not any handheld cardio first-aid kit that people, especially those at high risk of AIM, could continuously have on hand, and in the event of a symptom, they could make a preliminary diagnosis of AIM, and to pass on the results to a long-distance to be evaluated by medical personnel, and then, in the case such instructions were given they could take the appropriate anti-AIM medicament from the first-aid kit and to make the chemical test for AIM, whereby, they could speed up the healing process substantially. Such person could use the cardio first-aid kit also for prevention.

Devices for sensing of a multi-lead ECG, allowing monitoring of the chest leads, are not available in some handheld variant, they are bulky, in some stationary variant, or they are complicated in their permanent installation of electrodes that are fixed to the body by gluing, clips, belts, or suction pads, and setup of such device, as well as evaluation are complicated, they are designed for medical facilities and they are not suitable for use as handheld monitoring devices carried by the monitored person. There are not any simple handheld devices for short-term sensing of the pulse rate curve by some one-lead ECG device for preliminary diagnosis, which diagnosis would be understandable also for a layman, for example also for the monitored person, which, in case that some arrhythmia, or elevation of the ST segment, or other deviations from the normal ECG have been found, especially for detection of possible infarct, would allow to enlarge such device so that it would be able to provide multi-lead, for example up to twelve-lead, ECG record, to refine the diagnosis, preferably with the assistance of some professional medical personnel.

In the multi-lead ECG, there is not any device available, which could be applied simply and quickly to the body only by holding it with fingers during a short-term sensing of ECG, to avoid fixing of the electrodes to the body permanently, for example by suction pads, wristbands, braces, glue, belt, or by other mechanical means, what makes the ECG sensing longer, and makes it more complicated, more expensive and causes that the installation, uninstallation, and storage of a greater number of bulky parts are more complicated.

There is not any simple equipment for monitoring of cardiac activity, for example for sensing of the pulse rate curve, which could be enlarged to serve also for sensing of ECG, or enlarged to such an equipment for sensing of the less-lead ECG, for example of the one-lead ECG, which could be modified to an equipment for sensing of a multi-lead ECG when it is necessary, for example for the purpose to find deviations from the normal state. In most cases, a simple monitoring of the cardiac activity is sufficient, and it would be unnecessary to use a multi-lead ECG right at the beginning, because the initial detection of ECG deviations from the normal state, especially of the arrhythmia, can usually be detected with the curve of the pulse rate and from the elevation of the ST segment for detection of an infarct with the method described in this application, already with the one-lead ECG. To use a multi-lead ECG already for determination of the preliminary diagnosis would be inappropriate due to complexity, energy demands, larger dimensions of such equipment because a larger accumulator is needed, and due to time-consuming operation, despite the fact that it would be difficult for a layman, i.e. an ordinary monitored person, to operate it.

If necessary, for example, to exclude or confirm an infarct, recording of chest leads, preferably on a multi-lead ECG, is necessary, and its application is necessary immediately at occurrence of the first symptoms to save life. A multi-lead ECG, provided with the feature of recording the chest leads that could serve as an enlargement of a simple one-lead ECG, or of an equipment for monitoring of the pulse rate, and which would be of a handheld (pocket) design, does not exist in the prior art. Also, there is not any handheld design of a multi-lead ECG device for sensing of the chest leads V1 to V6 that persons with risk factors could always have with them, and if necessary, perform a multi-lead ECG with chest leads, and then send it from wherever they are right now, for example through a network of a mobile operator, to be evaluated by medical personnel, for example to the rescue service team, or to the monitoring center.

Also, there is not any equipment, which would allow some simple monitoring of the cardiac activity, for example of the curve of cardiac pulse and/or of a less-lead ECG, which could be, in case of necessity, able to provide a more detailed diagnoses, modified rapidly to a multi-lead ECG, especially to monitor the chest leads in case of a suspected infarct. Also, there is not any simple equipment of handheld design for monitoring of the cardiac activity, for example with a single-lead or two-lead ECG, which would allow to provide ECG of a chest lead, and which could be observed live by the monitored person or by medical personnel.

For a full assessment of the threat or occurrence of an infarct, it is necessary to provide a complete ECG record, what that is one of 21 leads. For such sensing, it is necessary for a device according to the state of the art to provide at least 10 conductors, by which the signals are fed from the body of the monitored person to the ECG device. Connection of these wires to the patient's body with suction cups, clamps and/or glued probes is uncomfortable and restrictive in movement, i.e. unsuitable for longer measurements and measurements at home or in the field. However, one-lead ECG devices are available on the market, which, if adjusted for the multi-lead ECG option, could correct the above shortcomings. For monitoring of more leads than as it is up to now possible, a 12-lead ECG could also be adjusted to a 21-lead one. This is necessary to exclude the myocardial infarction. Furthermore, 7-lead Holters or hospital bed monitors could be supplemented with additional leads, which would speed up the diagnosis of a possible infarction. Such devices are not known from the prior art.

### SUMMARY OF THE INVENTION

The shortcomings of the prior art are solved by the device according to the invention. The substance of the invention is a device for a multi-lead ECG, which uses a low-lead monitor, which, to provide sensing of a greater number of leads, is switched from the primary electrodes allowing sensing of less leads, to the secondary electrodes, what allows sensing of more leads. Preferably, the low-lead monitor is connected to the primary electrodes allowing to sense cardiac signals for processing by the monitor to data for a certain number of leads, which number is limited by the number of electrodes and/or by the monitor performance, which performance is determined mainly by the number of input amplifiers, the so called Front ends. For processing of a higher number of leads, the monitor is switched from sensing from the primary electrodes to sensing from the secondary electrodes, or it is moved into a sensing unit with secondary electrodes for preferably successive sensing from a greater number of electrodes and/or circuits, preferably located in the sensing unit, which electrodes are connected to the monitor successively, preferably in sets, through a connecting field and/or a relocateable electrode/electrodes is/are used for successive sensing of cardiac signals in spots determined for sensing by the chest leads.

The equipment for increasing of the number of ECG leads comprises circuits, preferably for forming of one of the following: a Wilson's central terminal, a strengthened Kranz's central terminal, and a pseudo-Kranz's central terminal.

The equipment for increasing of the number of ECG leads preferably comprises electrodes for sensing of cardiac signals, preferably it is formed by at least two primary electrodes, which are connected to the monitor, and by secondary electrodes, to which the monitor is connected from the primary electrodes, or which are connected to the primary electrodes. Further it comprises a sensing unit, comprising at least two electrodes, preferably formed by secondary electrodes, for sensing of cardiac signals and/or circuits, which unit is adapted for connection to the monitor instead of the primary electrodes, and further it comprises connecting elements and/or switching elements for switching between sensing from the primary electrodes and sensing from secondary electrodes, which are located in the sensing unit, further it comprises a monitor for processing of cardiac signals sensed at least from two primary electrodes to digital data for displaying of at least one ECG lead, which is adapted for switching to sensing from secondary electrodes, which are located in the sensing unit, and further it comprises a memory for storing of digital data, processed by the monitor from the analog cardiac signals sensed by the basic electrodes and/or by electrodes, which are located in the sensing unit, communication circuits for wired or wireless transmission of digital data, which transmission is live, or from the memory, to at least one cooperating unit, in which, or on which, the monitor is located, or cooperating units located remotely from the monitor, at least one such cooperating unit located remotely from the monitor, or a cooperating unit in which or on which the monitor is located, which unit is adapted for displaying of the ECG curves produced from the transmitted digital data via the communication circuit live or from the memory and for successive or simultaneous data displaying.

The electrodes are located on one of the following: on the monitor, on the cooperating unit, in which or on which the monitor is located, on a cooperating unit, which unit is located remotely to the monitor and is connected by a cable and by a connecting element to the monitor, on the sensing unit.

The equipment comprises a connecting field, preferably remotely controllable, which field successively connects the sets of electrodes and/or circuits of the sensing unit to the monitor with the objective to increase the number of ECG leads, which can be obtained from the cardiac signals sensed with electrodes against the leads, which can be obtained by sensing from the basic electrodes, wherein, the increase is reached by successive sensing of cardiac signals from sets of electrodes and/or circuits, located in the sensing unit, successively connected through the connecting field for successive processing by monitor.

Further, the equipment according to this invention preferably comprises one relocateable electrode for its successive application to the places designated for sensing of the chest leads, for their successive sensing and subsequent processing of the cardiac signals by the monitor.

The sensing unit is adapted for successive relocation of at least one relocateable electrode to spots determined for sensing of cardiac signals from the chest leads for their processing by monitor to digital data for displaying of the chest ECG leads.

Preferably, the monitor is connected to electrodes located on the monitor or on the cooperating unit, in which the monitor is located, what allows one-time sensing of cardiac signals and switching over to the electrodes located in the sensing unit, preferably designed in the form of a chest belt, so that long term or continuous sensing is reached.

Preferably, the monitor is located independently or in/on the cooperating unit for one-time or short-term monitoring with sensing from such electrodes, which are distant from the monitor and which are connected to the monitor by a cable and by connecting elements, which electrodes are designed for attaching to the chest, hands or fingers of the patient's body and it is adapted for long term or continuous monitoring by disconnection of the connecting element and by connecting of another connecting element by a cable, which leads to the electrodes located in the sensing unit, preferably designed in the form of a belt.

Preferably, the monitor is adapted for relocation with the connecting elements between the sensing unit with electrodes and the sensing units with a higher number of electrodes, than what is the number of primary electrodes, and for sensing of a higher quantity of cardiac signals by a higher number and/or type of electrodes allowing to process cardiac signals by the monitor to a higher number of the ECG leads, than what allow the primary electrodes, or in case of a monitor located in the sensing unit with electrodes adapted only for the one-time or the short-term testing by attaching of electrodes to the body, by relocation of the monitor into the sensing unit with electrodes, preferably in the form of a belt, the long term or continuous sensing is made possible.

Further, the equipment for multi-lead ECG according to the invention comprises a memory, which is adapted for successive storing of digital data, thereafter processed successively by monitor from the analog cardiac signals, which are sensed by the electrodes connected successively to the monitor via the interconnection field.

The equipment for increasing of the number of ECG leads, using a low-lead monitor, comprises circuits formed by one of the following: by circuits for forming of a Wilson's central terminal, what are preferably resistors; by a module for a Kranz's central terminal, preferably formed by an electrode for the Kranz's central terminal; by an electrode for chest leads; by circuits for a strengthened Kranz's central terminal, preferably formed by three resistors, which are connected to RA, LA, the Kranz's central terminal; by circuits for a pseudo-Kranz's central terminal, preferably formed by two resistors; by circuits for a pseudo-Wilson's central terminal, preferably formed by resistors connected to RA, LA, LL.

The connecting field is adapted for disconnecting of the primary electrodes from the monitor and for connection of sensing unit to the monitor for switching of the sets of electrodes and/or of circuits of the sensing unit to the monitor.

The connecting field is adapted for connection of electrodes comprised in the sensing unit to the monitor, which electrodes are a set of the primary electrodes and/or circuits, which are connected to the monitor primarily and a set of secondary electrodes and/or circuits, which are connected to the monitor secondarily, and preferably of the tertiary and further sets of electrodes and circuits, which are successively connected to the monitor, wherein, the sets have optional, preferably different number of electrodes and/or circuits for sensing of cardiac signals for processing by the monitor.

The sensing unit is adapted for successive relocation of at least one relocateable electrode to places determined for sensing of cardiac signals for the chest leads for their processing by the monitor to digital data for displaying of the chest ECG leads.

The connecting elements are adapted for switching over of the monitor from sensing with primary electrodes, which are located on the monitor or on a cooperating unit, in which the monitor is located, to sensing from electrodes or circuits in the sensing unit, wherein, preferably, the connecting elements are formed by spring contacts and are connected to electrodes and/or, preferably, the connecting elements are formed by a connector connected to the monitor and the connecting elements are connected by conductors to the electrodes in the sensing unit, preferably through a connecting field.

The connecting field is adapted for switching over of the monitor from the set of the primary electrodes to the set of the secondary electrodes or circuits, and preferably, to a tertiary or further set of electrodes comprised in the sensing unit, and the connecting field is connected between the monitor and the sensing unit, and it is controlled by a controlling element of the connecting field, preferably remotely.

The connecting field is adapted for initial interconnection with a set of a selected number of primary electrodes and/or circuits to the monitor for sensing of analog cardiac signals for the initial processing to digital data to at least one ECG lead, and after the initial processing of the cardiac signals by the monitor and after securing of the digital data by saving the data in memory and/or transmission of the data via a communication module into the cooperating units. Preferably, the connecting field is adapted for the second interconnection, and this of the second set of secondary electrodes, and/or of circuits to the monitor for the second sensing of cardiac signals for the second processing to digital data by the monitor to at least one ECG lead and after the second processing of the cardiac signals by the monitor to the ECG leads and securing of digital data. Preferably, the connecting field is adapted for the third and further interconnection of the third and further sets of electrodes and/or circuits to the monitor.

The switching elements, preferably formed by a switch, are adapted for switching over of the monitor from the primary electrodes to the electrodes in the sensing unit by disconnection the primary electrodes and connecting of the monitor to the electrodes in the sensing unit.

The connecting field is adapted for switching over of the monitor from the primary electrodes to the secondary electrodes in the sensing unit, and preferably also for switching of the sets of electrodes of the sensing unit to the monitor, wherein, the connecting field is connected between the monitor and the sensing unit and it is controlled by a controlling element.

The memory is adapted for successive storage of digital data, successively processed by the monitor from the analog cardiac signals sensed by the primary, secondary, and preferably further sets of electrodes for subsequent transmission via communication circuits for transmitting of digital data successively stored in the memory, for their evaluation and/or for displaying of a ECG curve in at least one cooperating unit.

The communication circuits are adapted for live transmission of digital data processed by monitor or for transmission from the memory of digital data, successively processed by monitor and stored in memory, to at least one cooperating unit, which is adapted for live displaying of a ECG curve corresponding to live transmitted digital data processed by the monitor, or the cooperating unit is adapted for successive or simultaneous displaying of an ECG curve corresponding to the transmitted digital data transmitted successively or simultaneously via the communication circuits.

The independently located monitor is connected to at least two primary electrodes located on the monitor or the monitor located on/in a cooperating unit is connected to at least two primary electrodes located on the cooperating unit. The switching over from sensing from the primary electrodes, which are located on the monitor or on the cooperating unit, for sensing from electrodes of the sensing unit is carried out by connecting elements directly to the monitor or via the electrodes located on the monitor or on the cooperating unit.

The connecting elements for interconnection via the connecting field directly to the monitor are formed by a connector and for interconnection via electrodes, the connecting elements are formed by flexible contacts on the electrodes, which are located on the monitor or on the cooperating unit, in which the monitor is located.

The monitor is adapted for connection of primary electrodes for sensing of cardiac signals for at least one lead, and it is adapted for switching over to the sensing unit, which is adapted for successive sensing from the sets of electrodes for displaying of more leads, than what is the number of leads for which the primary electrodes are adapted.

The monitor is connected to the primary electrodes located externally with regard to the monitor, or on the monitor, or on the cooperating unit, on which or in which the monitor is located, wherein, the monitor is adapted for switching over of the monitor to the electrodes of the sensing unit via the connecting field.

The connecting field, which is located within the monitor or the cooperating unit, in which or on which the monitor is located, is adapted for switching of the monitor for sensing of cardiac signals from the primary electrodes for sensing from electrodes of the sensing unit via the connecting elements.

The monitor is located in the cooperating unit, preferably formed by smart watch, for displaying of ECG from the digital data processed by the monitor from the cardiac signals sensed by primary electrodes, which are located on the smart watch. The monitor is adapted for cooperation with the smart watch, and switching over from sensing from the primary electrodes to sensing from the electrodes, which are located in the sensing unit via the connecting field with the connecting elements connected to the primary electrodes located on the cooperating unit, preferably formed by the smart watch.

The monitor, which is located in the cooperating unit formed by smart watch, is inter connected for sensing of cardiac signals to two primary electrodes located on the smart watch, and to electrodes for a wrist for sensing from a wrist, and the electrodes for sensing from a finger, and the monitor is adapted for connecting via the connecting elements and the connecting field for sensing from the sensing unit for successive connecting of sets of electrodes, which are located in the sensing unit for increasing of the number of two primary electrodes, which are primary connected to the monitor, to a higher number of electrodes successively connected to the monitor via the connecting field, connected from the sensing unit for an increased number of the ECG leads, which the monitor can process successively from the cardiac signals from the connected electrodes of the sensing unit, in comparison to the original one ECG lead, which the monitor is able to process from two primary electrodes.

Preferably, the connecting field is located on the base, which base is placed under the smart watch during sensing by the sensing unit for fixing of the base by pushing the smart watch by the force exerted by the strap and for isolating of the electrodes from the wrist, or the connecting field is located in/on the sensing unit and to the electrodes of the smart watch it is connected by a cable, preferably via the base.

The connecting element for connection of the electrode for a wrist is formed by a flexible contact and for connection of the electrode for a finger it is formed by the contact of the base, wherein, connecting elements are located on the base, which base for connecting of electrodes is placed temporarily under smart watch.

For fixing, the base is inserted under the smart watch temporarily, where it isolates the electrode for a wrist from this wrist, and connects it to the connecting field with a connecting element together with an electrode for a finger with another connecting element for sensing from the sensing unit. For sensing from the electrodes located on the smart watch, this base is removed.

The other contact of the base is located on the sliding part of the base, which base is shifted to the smart watch, for switching over to one from the electrodes of the smart watch, whereby, preferably, it is connected to a contact of the base, which contact contacts the other contact of the base by connecting it to the connecting field, or is connected firmly with the base, and the smart watch is put on the base, preferably from above.

For displaying of the ECG leads, the cardiac signals sensed by electrodes are processed to the digital signals by the monitor, at least one lead from the leads: I, II, V1 to V6, V1R to V6R, V7 to V9, wherein, optionally, four leads, III, aVR, aVL, and aVF, are calculated.

Preferably, the monitor is formed by the monitor for attaching, which is provided with electrodes formed by the primary electrodes, which are formed by the electrodes located on the surface, and the monitor is adapted for sensing from the primary electrodes by attaching of fingers to the monitor or of the monitor to chest. The monitor is adapted for sensing from electrodes of the sensing unit by interconnecting to the external connecting field with connecting elements formed by a connector and a cable, or it is connected by connecting elements formed by spring contacts, preferably located on a holder, which contacts are connected to the primary electrodes and they are connected by the spring contacts with a cable to the external connecting field, or they are located on the holder, and the connecting field is interconnected to the electrodes of the sensing unit, from which electrodes, preferably, they interconnect the sets of electrodes successively for successive sensing of cardiac signals for their processing by monitor to more ECG leads, than what is the number of them the monitor is able to process at one time from the primary electrodes. Preferably, the sensing unit comprises an electrode formed by a relocateable electrode, which is used for successive sensing of the chest leads.

The sensing unit is formed by at least one part of the following ones: a base, a base for the chest leads, a multi-electrode base, a monitor base of the monitor for the multi-lead sensing ECG with and small-plate of two electrodes or a plate of leg electrodes with three electrodes, a monitor base, abdominal, three-channel, with an electrode of a small-plate of two electrodes the second, a multi-channel base with and small-plate of two electrodes chest or a plate of the chest electrodes, a base of the universal monitor, a base for RL, a base for RL and twice V, an elongated base, a base with the reference, a complete base, a base in the form of a belt, a base, a base for LL, RL with glued electrode.

The monitor is located in the unit with external electrodes formed by external primary electrodes, which are connected by a cable via the connecting elements formed by a connector and the connection to the sensing unit will be carried out by disconnection of the primary electrodes by disconnecting of the connecting element, through which they are connected to the unit, and by connecting of another connecting element with the connected sensing unit via the connecting field for successive sensing of cardiac signals for their processing by the monitor to more ECG leads, than what the monitor processes from the primary electrodes.

The unit with external electrodes is formed by an ECG device or by a Holter device according to the prior art.

The connecting elements are adapted for switching over between sensing from the primary electrodes and sensing from the secondary electrodes of the sensing unit by connecting of the sensing unit to the monitor instead of the primary electrodes by connecting elements, preferably formed by a connector or by spring contacts.

The monitor is adapted for relocation from one sensing unit, which preferably senses cardiac signals with primary electrodes into a second sensing unit, preferably another sensing unit with secondary electrodes for sensing of cardiac signals for more or for different leads, than what allow to sense the primary electrodes.

Preferably, the monitor is moved from the sensing unit with the primary electrodes on the unit, which allows continuous sensing, preferably formed by a chest belt or on a sensing unit, which comprises a relocateable electrode for sensing of the chest leads, or into a base formed by a chest belt, which unit allows sensing of cardiac signals for more leads, than what allows the sensing unit with primary electrodes.

Further drawbacks of the state of the art are removed also with equipment according to the invention, which equipment is provided with electrodes. For short-term sensing the equipment is applied to body, preferably in the area, which is recommended for sensing of the chest leads V1 to V6, and preferably it is handheld, preferably it is held by fingers. Preferably, a pocket ECG for preliminary diagnosis is adapted for sensing of a simple, low-lead, preferably one-lead ECG and in case of need, for example when deviation from the normal state is found, or in case of heart problems of the monitored person, it is possible to adapt in a simple way the monitoring for a multi-lead one, preferably up to 12-lead ECG, preferably for the detailed diagnoses of the AMI.

This equipment is adapted for optional enlargement, preferably from the one-lead up to a multi-lead, preferably up to the twelve-lead ECG.

For the one-lead ECG sensed from fingers or from chest, preferably a simple, cheap mono-channel monitor is used, which monitor is preferably fastened to the sensing base of a universal monitor with two electrodes, to which the chest is applied or to which both hands are applied, preferably fingers of both hands.

For sensing of the chest leads with the so called Kranz's central terminal, the base is applied to the chest, preferably centrally, in a spot under the nipples, with one electrode applied to the chest for sensing of the pseudo-V4 signal in the spot for sensing of the V4 signal, and the other electrode is applied in the area for sensing of the pseudo-Wilson's central terminal, called the Kranz's central terminal, which is located on the chest centrally, where cardiac signals from the places of the right (RA), the left hand (LA) and the left leg (LL) are led by resistance of the human body, and these signals form imaginary area of their electric center, centrally on the chest.

For sensing of the pseudo-V5 lead the base is shifted in the direction to the left, so that the left side electrode of the base is located in the place, which is recommended for sensing of the lead V5, and the right side electrode of the base is located in the area of the Kranz's central terminal.

To make possible sensing of the chest leads V1 to V6 with the base without using a calculation or resistors of the Wilson's central terminal, one of the electrodes of the base, the so called Kranz's electrode is placed on the chest to the plane below the nipples, where a new area is created in a natural way, named after its inventor as the area of the Kranz's central terminal, where the Kranz's central terminal is placed, which senses through the resistance of the human body the signal from the sensing points for RA, LA and LL, and it is formed by resistances of the human body, approximately in the center from the places for placing of the RA, LA, LL, from which voltage is applied to the Kranz's central terminal.

The other electrode of the base, the electrode for sensing of the pseudo-V leads, is placed in the plane under the left nipple, for example to the place for sensing of the required lead V5. Preferably, it is possible to place the pseudo-V electrode for sensing of other chest leads V1 to V6 successively to places recommended for sensing of V1 to V6, wherein, the first clip of the monitor is placed into the position in the area of the Kranz's central terminal.

The so obtained pseudo-V4 and pseudo-V5 leads are not accurate, but they are sufficient for a quick orientation, whether the ST segment is elevated to determine the possible risk of an infarct.

In case of preliminary diagnosis, that an infarct is suspected, or in case of subjective symptoms of an infarct felt by the monitored person, the monitoring equipment is adaptable to be modified for a more accurate sensing of the chest leads with the Wilson's central terminal. For short-term sensing of leads V1 to V6 from the chest electrodes V1 to V6, the signal of the Wilson's central terminal is applied to the input circuit (Front-end) together with the signal from the electrodes located in the places for sensing of the leads V1 to V6. The signal, in the case of the mono-channel monitor from the Wilson's central terminal, which terminal is formed as the electric center from the signals sensed by electrodes in the places recommended for sensing of the leads RA, LA, and LL, and brought together into one point through the resistors.

The places for sensing of the leads V1 to V6 from the chest electrodes are distributed in a standard way in the area of the chest, and to the V1 and V2 symmetrically on the edges of the sternum at the level of the fourth ribs and the V3 to V6 are located in the curve to the left, falling below the level of the fifth rib.

For forming of a conventional Wilson's central terminal resistors connected to the electrodes for sensing of the RA, LA, LL, and for the reference potential, preferably the electrode for sensing from the right leg RL are used, or the reference potential is formed artificially. The electrodes RA, LA, LL, and RL are located on the limbs. Preferably, the shifted RA electrode, the shifted LA electrode are used alternatively, which are located in the areas of the right and the left shoulder holes, and further, the shifted LL electrode, and preferably, for the reference potential the shifted RL electrode. The LL electrode is located on the left side and the RL electrode on the right side of the abdominal area.

For the implementation of a conventional electrode or a shifted Wilson's central terminal, the signals from all three electrodes RA, LA, LL are fed through resistors of the Wilson's central terminal, or the signals are calculated from the signals of the electrodes RA, LA, LL.

For the short-term sensing from a wrist for sensing of the I curve of the ECG, for example for finding of arrhythmia, it is possible to move the mono-channel monitor to a holder on the wrist, which is fastened with a wristband, where on the lower part of the holder, which rests on the wrist, is located an electrode sensing cardiac signals for ECG from the first arm, and preferably, the left arm LA, and on the perimeter of the holder or on the monitor is located an electrode sensing cardiac signals from the other arm, preferably from fingers of the other hand, preferably the right arm RA.

For a short-term sensing of the ECG for the chest leads, for example for detection of an infarct (AIM), preferably, the mono-channel monitor is moved on the holder for a multi-lead ECG, which holder is pressed to the chest by one, preferably the right arm (RA), preferably by fingers applied to the electrode for the right arm (RA) on the top surface of the of the holder, whereby the chest electrode Vx is pressed to the chest on the other side of the holder, which rests on the chest, for the chest electrodes (leads) V1 to V6. By moving of the chest electrodes Vx leads V1 to V6 are successively sensed.

The other arm, preferably the left one (LA), holds the top surface of the small-plate of the leg electrodes, whereby it presses the electrode of the left leg (LL) in the left side of the abdomen area. Preferably, the electrode of the left arm (LA) is located centrally on the small-plate and from the other side are located, preferably symmetrically, the y electrodes for the left leg (LL) and the right leg (RL), which serve preferably for reference signal supply. From the electrodes for LA, RA, LL the Wilson's central terminal is obtained, against which the chest leads are sensed. For sensing of the ECG leads I and II, the switching members are added, preferably formed by switches, which are switched, preferably manually for a multi-lead ECG sensed by a mono-channel monitor with one input of an individual electrode to the monitor for targeting of the sensing of the curve I of the ECG from LA and RA, of the curve II of the ECG from RA and LL, wherein, the curve III of the ECG, as well as the curves aVR, aVL, aVF are preferably calculated from the curves I and II of the ECG.

Thereafter, the switching members are switched to the position for successive sensing from the chest electrodes V1 to V6.

In case of the mono-channel monitor with one input circuit (Front-end), the cardiac signals are sensed for individual leads of the ECG of the type I and II successively with two electrodes, i.e. the lead I with the electrodes RA and LA and the lead II with the electrodes RA and LL. The leads III, aVL, aVR, and aVF are preferably calculated from the leads I and II. The chest leads V1-V6 are sensed successively with a single electrode “V” (V1 to V6) by attaching it to the chest in places for sensing of the chest signals V1 to V6, and the signal of this electrode is fed to one input of the monitor and to the other input of the monitor is fed the signal from the Wilson's central terminal, which terminal is obtained with three resistors as the electric center of signals from electrodes for sensing of leads RA, LA and LL or from electrodes RA, LA and LL shifted for sensing of pseudo-leads, or against the Kranz's central terminal.

Preferably, the reference is sensed from the RL.

Preferably, before a short-term sensing of a certain lead, the switches are switched to the appropriate position, the electrodes are applied respectively, and the push-button “Start” of the record is pushed for displaying of the sensed lead and/or for saving in memory. So, all leads sensed will be displayed successively and/or saved in memory. Displaying is carried out in local or distant units. It is optional, which leads will be displayed simultaneously live or from the memory and which will be saved.

The equipment with mono-channel input allows displaying of only one for the simultaneously sensed leads. Successively, up to twelve leads can be displayed live. From the memory, it is possible to display only the full-valued twelve-lead ECG simultaneously from different sensed time periods.

Data transmission from the monitor to the cooperating near units of the evaluating and displaying equipment runs through a local link, preferably realized as a wired link or a wireless one, some Bluetooth, Zig Bee, or WiFi and to remote cooperating units with a long-distance link, preferably through a mobile operator's network.

The monitor is adapted for relocation from one sensing member to another one, preferably to the base or holder, as needed, for sensing of the mono-lead to twelve-lead ECG. The monitors are connected to the sensing members mechanically and electrically with a holding mechanism, preferably comprising connectors also for the electric connection.

Preferably, the sensing units are provided with switches for preferably successive switching over of the appropriate electrodes to the inputs of the monitor.

Optionally, the monitors are adapted for simultaneous sensing of one, two, three, or up to eight channels, and they sense the selected leads from the leads I, II, V1 to V6. The leads, which remain up to the complete twelve-lead ECG, i.e. the leads III, aVR, aVL, and aVF, are preferably calculated, or they are obtained electronically.

Preferably, instead of individual switches for connection of appropriate electrodes to the monitor the push-buttons or rotatable switch are used for switching of individual switches. In its individual positions the leads are marked, to which it will be switched over, and which switch the respective switches for sensing of the required leads.

Preferably, switching over of the switches is automatic, wherein the control unit switches over the switches successively for sensing of the appropriate leads and displays switching over with a LED or in the display or acoustically. Start of the sensing can be switched on after setting up of the switches by the push-button Start, or automatically in adjustable intervals or after attaching of electrodes is detected.

A multi-channel monitor, for example provided with two channels, i.e. with Two input circuits (Front-end) allows to sense and to display live or save in memory two chest leads simultaneously, for example V4 and V5 against the Kranz's central terminal with the base. In case of sensing the chest leads V1 to V6 against the Wilson's central terminal with the base for the multi-ECG lead preferably always two leads, i.e. triple sensing are made for live displaying or saving in memory, for example V5 and V6, V3 and V4 and V1 and V2. To obtain the remaining six leads, preferably, the holder switches the two-channel monitor to sensing from the electrodes for the right arm RA and the left arm LA through the first channel and of the right arm RA and the electrodes for the left leg LL through the second channel for simultaneous sensing of the leads I and II, wherein, preferably, four leads are calculated from them, and to the leads III, aVF, aVR and aVL. When the processed leads are saved in memory successively, it is possible do display all twelve leads successively or simultaneously and/or only the selected leads.

When a three-channel monitor, i.e. one provided with three input circuits (Front-end), is used, it is possible to obtain a twelve-lead ECG already with triple sensing, and/or to sense leads I, II, and one from the leads V1 to V6 simultaneously. Other leads V1 to V6 can be obtained by switching over of the leads V1 to V6 to input of the monitor or by manual relocating of the electrodes for leads V1 to V6. The leads III, aVF, aVL, and aVR can be calculated, and thereby obtained successively for live displaying a/or saving in memory of all twelve leads. Also, they can be displayed at once or successively from the memory.

Preferably, for common monitoring of the cardiac activity, also the pulse curve is displayed with limits of regular pulse, wherein, preferably, for preliminary information about pulse regularity, omitting of a pulse and about other phenomena, it is displayed for a longer time, for example for 30 seconds. This is an advantage in comparison to an ECG, where mostly a shorter time period is displayed including only several pulses, about 5 to 10.

In case of use of a three-channel monitor, it is possible to sense simultaneously and display live and/or save in memory three leads and to calculate from them four other leads. Preferably, it is possible to sense from electrodes for RA, LA, and LL simultaneously and preferably take the reference potential from the RL. From these leads, it is possible to obtain the Wilson's central terminal with resistors or by calculation simultaneously. From the mentioned electrodes, leads I and II can be obtained, and 4 leads are calculated. At the same time, with the holder, it is possible to display live or save in memory three leads, and it is possible to make some selection from twelve leads or from those, for which the holder is provided with electrodes. Recording or live displaying of all twelve leads of the holder, if you make triple switching.

In case of the eight-channel monitor the holder allows live transmission or saving in memory of twelve leads, of which four leads are calculated.

The monitors for the short-term monitoring, preferably adapted for universal use also for the long term monitoring in case of need, are fastened by holders on the body of the monitored person by fastening members, preferably formed by belts. For the mono-channel monitor, preferably, the base is fastened to the chest for sensing by one electrode, for example in the area for sensing of the lead V5, wherein, the other electrode is located in the area of the Kranz's central terminal. For sensing with V4, the base is shifted to the right.

For a long-term sensing of associated cardiac signals from the lead I and the chest leads, preferably the electrodes on the chest belt or the sensing unit are made longer so that they sense from a larger area.

This brings an advantage also in displaying of the segment ST for assessment of its elevation in simple equipment with mono-channel monitor on the chest belt with two electrodes.

Preferably, the curve of cardiac pulse is monitored, wherein, data information about individual heartbeats is transmitted, from which individual points of the curve are calculated and displayed. By not transmitting data to create an ECG waveform, which would be more energy intensive, accumulator power is saved and the monitor can be implemented as smaller.

Cardiac activity is assessed from the pulse curve, and in case of need, for example when a deviation from the normal is found, for example in case of arrhythmia, or in case of symptoms indicating incorrect cardiac activity, short-time ECG is monitored. For long-term monitoring, preferably, additional accumulator is connected to prolong the maximum time period of monitoring with respect to the capacity of the power supply. During monitoring of the pulse curve, preferably, the ECG is continuously recorded on the storage medium of the monitor, which medium is preferably movable and preferably formed by SD card, from which the complete ECG curve can be transferred.

Monitoring of the pulse curve instead of the ECG is preferable in that the diagnosis is simple, especially for laymen and allows recording and display of a longer section of pulses than the ECG.

Preferably, elevation of the ST segments is sensed, evaluated in the monitor, even when, for example, the chest lead data is not transmitted to the cooperating units of the evaluating and displaying equipment, preferably a mobile phone, to save energy, and the monitor will sound an alarm in case of elevation of the ST segment for switching to the ECG monitoring in the cooperating units also for transmission of the chest leads into the evaluating and displaying equipment for confirmation of the diagnoses. As necessary, it is possible to provide the scanning units with differently equipped monitors with regard to the number of channels, and create sets from them that are equipped differently, as needed.

An example of how to use the set is for monitoring of the pulse curve with limits of the regular pulse, arrhythmia and variability using a small in size and energy-efficient monitor and with the appropriate sensor unit, which are parts of a set, and in case of arrhythmia occurrence, which preferably, the monitor reports by an alarm, the arrhythmia is checked in the displayed ECG.

In case of symptoms or during automatic evaluation of elevation of the ST segment, what preferably is carried out in the monitor even when only pulses are transmitted to the energy saving display device to form a pulse curve, it is possible to it is possible to pass to chest leads monitoring, preferably, to twelve-lead ECG for checking diagnoses of elevation of the ST segment.

For fitting of the monitoring device with variously equipped sensor units and monitors, the units from the set are used, which can be equipped as needed with appropriate units, which are interchangeable and adapted for cooperation using software.

Preferably, the results of monitoring are sent with a long-distance connection to the cooperating units for communication with remote participants, preferably with medical staff for ECG evaluation. In case of infarct risk, preferably, it is monitored continuously, preferably in a simple way with a chest belt, which has less leads, preferably using a mono-lead monitor, and a Kranz's central terminal, or the collecting electrodes, wherein, the ST segments, and in case of an elevation, when preferably the monitor sends an alarm, a detailed analysis is carried out on a to more-lead, preferably twelve-lead device. In this, it is possible to use a universal 2, 3 or up to twelve-lead monitor, which is used also for monitoring of the pulse curve and/or a less-lead ECG, when all channels of the monitor are not used or are used for processing of the ECG for recording to SD card. This saves energy and the chest belts or bases are smaller than for a multi-lead ECG.

In case of need of a multi-ECG lead, the monitor is moved into more complex bases or holders and more, preferably all channels of a multi-channel device, preferably of up to eight channel devices.

Fitting of the monitored equipment with various units and monitors as needed is carried out preferably using units that are parts of the set.

A three-channel equipment with a three-channel monitor uses electrodes for sensing of RA from the right arm and Vx from the respective place on the chest, which is recommended for sensing of V1 to V6, by attaching the base to the body of the monitored person.

Electrodes of the holder sense the signal from places recommended for sensing of RL, LL, LA signals, and pushing of the push-button Start activates recording or displaying or transmitting to the respective Vx lead, on which the electrode for sensing of the lead V is applied.

Successively, it is possible to display or save in memory the leads V1 to V6 simultaneously with the leads I, II, wherein, the leads III, aVL, aVR and aVF can be calculated. Therefore, in this way a complete twelve lead ECG is available.

The equipment is adapted for displaying of all twelve leads successively or simultaneously from the memory, or for printing, or for wire, or wireless transmission.

By using of the eight-channel monitor and by using of the base for simultaneous sensing of leads V1 to V6, the equipment will be adapted for simultaneous sensing of the leads I, II, V1 to V6 and for calculation of the leads III, aVR, aVL and aVF. The equipment is adapted to display, print, transmit, and store in memory these twelve leads or only the selected leads, as needed, simultaneously or successively.

Monitoring and evaluation of arrhythmia or an infarct with the monitoring equipment fastened with the fastening members to the body is carried out preferably for a short time or for a long time, periodically, by activation of the control unit of the monitor or by the monitored person or at the instruction of medical staff or when the monitored symptoms occur, or continuously. In the case of an infarction, the evaluation of the symptoms is preferably performed visually by comparing the recorded ECG curve of the monitored person with the curves showing the These comparisons can be performed by a monitored person, or automatically, preferably by a monitor control unit, which preferably activates an alarm in the event of an infarct.

In the case of an infarction, the elevation of the ST segment in the chest leads, and other deviations of the ECG from normal, which could indicate an infarct, are considered, for example a reversible T-wave.

In case of arrhythmia, preferably the curve of pulse rate with limits of irregular pulses is evaluated, especially the percentage of irregular pulses from the number of pulses, and in case that it exceeds certain adjustable limit, preferably, the control unit activates alarm, and preferably the presence of the wavelet P is verified to verify the sinus rhythm, and if it is absent, the arrhythmia type is assessed. The size limit of the arrhythmia for triggering an alarm is preferably set according to the history of the arrhythmia of the monitored person so as to exceed the value of the sinus arrhythmia, so that only other types of arrhythmias, other than the sinus one, are evaluated. In case of a monitored person with a normal sinus rhythm, the limit is preferably set above the sinus rhythm limit, i.e. 13% above the average heart rate. This means that every pulse, the recalculated value of which exceeds the deviation 13% from the average pulse rate within the set number of previous pulses or within the period of time, is considered as arrhythmia. Preferably, together with the curve of the pulse rate are displayed also the curves with limits of the sinus rhythm, that is ±13% off the average heart rate to facilitate the diagnosis of arrhythmia. At regular heart rhythms, heart rate variability is assessed, and if its value is below a certain adjustable value, an alarm is preferably triggered, and then the presence of a P-wave is assessed to confirm sinus ECG, or its absence indicates tachycardia or flutter. The first operative evaluation is preferably performed by the trained monitored person themselves, preferably from a heart rate curve, arrhythmia, and variability, or it is performed automatically by the monitor control unit, which unit triggers an alarm if arrhythmia is detected in the monitored person. The detailed diagnosis is carried out from the ECG, which diagnoses, the monitoring device advantageously forwards to the medical staff to confirm the diagnosis.

Preferably, the monitoring device is modular, what means that it is possible to choose a simple one, with less features for some initial informative monitoring, and it can be enlarged to equipment with more features or it can be substituted by such equipment with more features.

This is done by modification of the sensing equipment, or modification of the monitor, or by both.

For example, as the starting equipment for a preliminary diagnoses, a sensing equipment with a small-plate with two electrodes can be selected for sensing of one of the leads, preferably of the lead I of the ECG, by attaching the fingers or the chest lead V5 with the Kranz's central terminal, or of a combined lead by attaching a small-plate with two prolonged electrodes to the chest, fitted with a mono-channel monitor.

Preferably, the extension of functions is achieved with relocating this monitor on the sensing equipment equipped for successive sensing of more ECG leads, for example up to twelve leads. By swapping for a multi-channel monitor, for example a two-channel one, two channels can be sensed at once, by swapping for a three-channel, 3 channels can be sensed at once, and the corresponding number of leads, for example leads I, II, and one chest lead, and by swapping for eight-channel monitor up to 8 channels can be sensed at one time, and the appropriate number of leads, for example the leads I, II, and 6 chest leads V1 to V6, and the other four leads, i.e. III, aVF, aVL and aVR can be calculated from leads I and II in all examples.

To take full advantage of the multi-channel monitor, an appropriate scanning device is preferably used, which allows scanning of all channels.

It is possible to use a multi-channel monitor and modify only the sensing equipment for a lowered number of channels, wherein, some of the channels of the monitor are intentionally not used when used in less-channel sensing equipment. The advantage is that from the beginning, it is possible to use more simple sensing equipment for lower number of channels, for the preliminary diagnoses, and only in case of need to use more complex multi-channel sensing equipment, which is more difficult to handle and into which the monitor is moved. An advantage is that into any sensing equipment, even with different numbers of scanned channels the same monitor may be used, and it is not necessary to have available more monitors for sensing of a greater number of different channels.

The monitoring equipment consists from a monitor and sensing equipment, and preferably it is universal and it is possible to fasten it also for continuous monitoring to the monitored person with fastening the equipment. This means that the sensing equipment, which is to be used for attaching to the fingers or to the body of the monitored person for short-term sensing, is adapted to be used for long-term sensing by connecting it to a fastening equipment, which holds it to the body of the monitored person.

It has the advantage of energy savings and simplicity of diagnosis, especially for lay people, as well as of the possibility of a longer recording of cardiac signals than with an ECG, and thus in the clarity of the occurrence of arrhythmias. The displaying of the curve of heart rate as a function of time with curves of regular heart rate limits, for example ±13% of the average heart rate instead of the ECG, is preferably used in the initial displaying of heart rate, and preferably, the ECG is displayed when deviations of the heart rate curve from normal are found. Savings are achieved in the case of wireless transmission from the monitor to the evaluating and displaying device, preferably a mobile phone, a smart watch, a PC, a server, by preferably transmitting only information on the occurrence of individual pulses, i.e. once per second at heart rate pulse 60/min compared to the transmission of the ECG curve, a larger amount of information, what places significantly higher demands on the power consumption of the monitor than how it is in the transmission of heart rate.

The heart rate curve is sufficiently giving evidence for detecting of the presence of arrhythmia and other deviations of cardiac activity from the normal state.

The ECG curve, the processing and transmission of which is energy-intensive, is preferably displayed only occasionally for inspection, in particular when deviations from the normal are detected with a heart rate curve.

This allows you to use an accumulator that is several times smaller than that needed for continuous ECG transmission.

If it is necessary to transmit the ECG continuously, an additional accumulator is preferably connected, preferably by a connector, to provide sufficient power for the continuous transmission of the ECG from the monitor to the cooperating unit.

Preferably, when transmitting only the heart rate to the cooperating units, the ECG curve is recorded continuously on a storage medium, preferably a removable one, preferably formed by an SD card, and it is adapted for subsequent transmission of the ECG curve to a PC and/or to a cooperating unit.

For long-term monitoring, it is possible to fasten it to the chest of the monitored person with a fastening device, preferably an embracing belt, for example a base of a universal monitor connected by bindings, preferably for monitoring the chest leads with a Kranz's central terminal. For continuous transmission of the ECG curve, an additional accumulator is preferably connected via a secure connector.

For continuous monitoring, a sensing device, preferably formed by a chest strap, can be applied to the chest of the monitored person for monitoring chest leads using a Wilson's central terminal. For simplicity, the electrodes for the chest leads V3 to V6 are placed on the chest strap together with the electrodes for the left and the right arm, LA, RA and preferably also for the right leg RL. Preferably, a monitor is also located on the chest strap. The electrode for the left leg LL is connected by a cable and it is located in the abdominal area on the left side, preferably on a hip belt, or it is held by a trouser belt. Alternatively, also the right leg RL electrode can be placed on the hip belt.

Alternatively, the electrodes for the left and the right hands, LA, RA, can be placed on the transverse straps instead of on the chest strap, where alternatively, also the electrode for the left and right legs, LL, RL, can be placed.

Alternatively, the electrodes for the left and right arms, LA, RA, are applied to the shoulder straps or wristbands.

Alternatively, the electrodes for the chest leads V1, V2, V3 are applied to the chest transverse straps on the sides of the straps, which extend up the chest, wherein, the side of the straps, which is below the chest strap provides that the electrodes are pressed against the chest by the chest strap.

The term Wilsons'central terminal (WS) is used by the professional public, wherein, in the historical context, this term at first meant actual physical clamps to which the heart signals from the monitored person's body were applied, namely from the places recommended for sensing potentials referred to as LA—the left arm signal, RA—the signal of the right arm and LL—the signal of the left leg, and this through resistors, in an optimized way, so that they are not affected by themselves and yet the appropriate signal strength is achieved.

By bringing together the potentials from three places, the Wilson's central terminal obtains the resulting potential, against which the chest leads Vx are sensed. The potential of RL right leg proved to be a suitable reference for measuring the chest leads.

However, new technologies have made it possible to combine LA, RA, LL signals only within integrated circuits, and thus the Wilson Wilson's central terminal is only a hypothetical point. However, it is still talked about it, as about measuring using a Wilson's central terminal, what addresses the method of generating a reference signal.

For measuring with this method, it is necessary to place the electrodes on the body of the monitored person in the places recommended for sensing of LA, RA, and LL, what always brings about certain degree of discomfort to the monitored person.

The effort in the solution according to this application therefore is to find a more comfortable solution by not installing the electrodes LA, RA, LL in the recommended places, which will have the advantage of easier and faster placement of electrodes, but at the cost of lower accuracy. The philosophy of the method is to start with a more comfortable, faster method of sensing, without installation of the electrodes LA, RA, LL, and only when deviations from normal have been detected, for example an elevation of the ST segment has been found, proceed to a less comfortable but more accurate method of sensing using the Wilson's central terminal with the necessary installation of the electrodes for LA, RA, LL. This less accurate method is realized using the Kranz's central terminal (KS), which terminal is similarly hypothetical as the Wilson' central terminal at present. It can be referred to as a pseudo-Wilson's central terminal.

It comes to existence by declaring the Kranz's central terminal area on the lower part of the sternum bone is declared to be the reference terminal instead of the Wilson's central terminal. The reasoning is such that from the spare points for sensing LA, RA and LL, i.e. the left arm shoulder pit, the right arm shoulder pit and the abdomen area in the left part, there is always approximately the same distance to the Kranz's area, and thus a similar amount of tissue resistance, i.e. of the internal resistances of the body, which substitute the resistors located externally with respect to the body, connected to the external electrodes for LA, RA, LL to the same points as the internal resistors of this Kranz's terminal. The difference between WS and KS is that for WS, the cardiac signals are sensed from the locations designated for sensing of LA, RA, and LL with external electrodes, which are routed via external resistors to a point called WS. For the KS, on the other hand, the signals from the LA, RA, LL sensing sites do not bring the internal resistances of the human body approximately to a point around the lower part of the sternum bone called KS, where the potential profile is similar to that at the Wilson central terminal, wherefrom the signal is sensed using the external electrodes. In contrast to it, for WS, the potentials LA, RA, LL are sensed by external electrodes and are fed to WS through external resistors. Thus, the internal resistors of the human body are used for the Kranz's central terminal, KS, which supply signals to the area of the sternum bone, for which it is sensed by an external electrode 143.

If we want to obtain a more accurate signal than when scanning with KS, we proceed to the first making more complex, namely we start scanning with pseudo-LA′, RA′ from spare positions on the sides, preferably located on a strip-shaped base, which are interconnected between themselves via two resistors, which create a pseudo-KS, and for even more accurate measurement of sensing through three external resistors on the KS, what creates an enhanced KS. A simplification of WS at the cost of accuracy is the connection of LA′, RA′ to LL′, or LL electrodes, making the pseudo-WS less accurate measurements than the WS obtained from the recommended sites for LA, RA and LL, preferably on the limbs. For the most accurate results, we sense LA, RA and LL from the recommended places on the limbs. Each refinement brings about more complexity of the scanning unit. For leads I and II, there are two methods of measurement, namely to measure leads I and II at once or sequentially. If measured sequentially, a mathematical method of selecting from the measured ECG section with the same distance R-R for leads I and II is used to calculate leads III, aVf, aV1, aVr and preferably form together an image of a conventional ECG.

For sensing of chest leads with WS, a WS sensing system is implemented by applying signals sensed by chest electrodes to one monitor amplifier input and signals from WS obtained by electrodes sensing LA, RA, and LL and fed to WS by external resistors to another input. The new system for sensing of the chest leads using KS applies (the new system does not apply) to one input of the monitor amplifier the signals from electrodes located on the chest and to the other input signals obtained with the electrode located in the lower area of the sternum bone.

### BRIEF DESCRIPTION OF THE DRAWINGS

FIG. 1 shows formation of a conventional Wilson's central terminal.

FIG. 2 shows the chest sites recommended for sensing of leads V1 to V6.

FIG. 3 shows formation of the area of the Kranz's central terminal.

FIG. 4 shows location of the base of a universal monitor mounted on the chest with a belt, used for sensing of the pseudo-lead V5 with a chest belt for the long-term sensing.

FIG. 5 shows location of the base of the universal monitor on a wristband for sensing of the lead I of the wristband

FIG. 6 shows a base for sensing of up to 12-lead ECG by switching of leads by attaching to the chest and of the electrodes LA, RA held with fingers. FIG. 7 shows the sensing of a multi-lead ECG by attaching to the chest and of electrodes LA, RA held with fingers.

FIG. 8 shows sensing of multi-lead ECG by attaching of electrodes held with fingers using a leg electrodes plate.

FIG. 9 shows block diagram of sensing of leads I, II, V1 to V6.

FIG. 10 shows a table for switching over of the measured leads.

FIG. 11 shows sensing always of two V leads at a time.

FIG. 12A shows a block diagram of a monitor, a monitor base, and an electrode plate.

FIG. 12B shows formation of a Wilson's central terminal.

FIG. 13 shows sensing with a two-channel abdominal base and of a small-plate with two electrodes.

FIG. 14 shows a 12-lead sensing using an electrode chest plate.

FIG. 15 shows a block diagram of an eight-channel monitor for 12-lead sensing.

FIG. 16 shows a base with a reference and connection of a small-plate, of a small-plates of an electrode on a cable, of a small-plate of electrodes for a two-channel monitor, and o a small-plate of three chest electrodes, and further placement of the base and the small-plates on body of the monitored person.

FIG. 17 shows a block diagram for connecting of the input circuits of a monitor with a taken-out reference.

FIG. 18A shows sensing of chest leads with a complete base, which base, after supplementing it with an expanding plate or with segments of electrodes, can sense all chest leads at the usual width of the chest belt.

FIG. 18B shows using of an expanding plate.

FIG. 18C shows using of a segment of electrodes for measuring of the leads on the right side of the chest.

FIG. 19A shows for illustration formation of a strengthened Kranz's central terminal and of a pseudo-Kranz's central terminal.

FIG. 19B shows formation of a pseudo-Kranz's central terminal.

FIG. 20A shows formation of a Wilson's central terminal and of a pseudo-Wilson's central terminal with the side and the shoulder electrodes and of the electrodes on the abdominal small-plate.

FIG. 20B shows an example of sensing with a Wilson's central terminal.

FIG. 20C shows an example of obtaining a Wilson's reference with a shoulder strap.

FIG. 21A shows a simplified base with an extension on an arm and its use I cooperation with the small-plates of electrodes for sensing with a Wilson's or a Kranz's central terminal and further a block diagram of connection.

FIG. 21B shows the formation of a pseudo-Wilson's central terminal.

FIG. 21C shows a block diagram of switching over between measurements with a Wilson's central terminal and a Kranz's central terminal.

FIG. 21D shows the measurement with slight turning of the base.

FIG. 21E shows use of a pull-on electrode.

FIG. 21F shows a block diagram of switching of the signals from electrodes to inputs of a monitor.

FIG. 22 shows a multi-electrode base with transverse straps with electrodes and side belts for sensing of a complete ECG.

FIG. 23 shows a wide chest belt, which can sense also chest leads V1 to V3 and V3R.

FIG. 24A shows sensing with a sensing unit on a belt with side electrodes, with side belts and with shoulder straps and with an electrode for LL on the strap of an abdominal electrode or with electrodes for LL and RL on clips.

FIG. 24B shows use of side belts.

FIG. 24C shows use of shoulder straps.

FIG. 24D shows standard method of sensing the RA, LA, and LL signals.

FIG. 25A shows schematically interconnection of sets of electrodes via a connecting field to the input circuits of a monitor.

FIG. 25B shows an alternative embodiment of the switching over.

FIG. 26 shows a sensing unit adapted to switch over between sensing with a Kranz's central terminal and sensing with a Wilson's central terminal, and a block diagram of the connection of the electrodes to monitor.

FIG. 27A shows a base with a reference allowing switching over from sensing with a Kranz's central terminal to sensing with a Wilson's central terminal, and a block diagram of the wiring.

FIG. 27B shows a block diagram of a sensing unit.

FIG. 27C shows sensing of chest leads using a Kranz's central terminal.

FIG. 27D shows sensing of chest leads in pairs.

FIG. 27E shows sensing with a Wilson's central terminal.

FIG. 28A shows the change from sensing with a Kranz's central terminal to sensing with a Wilson's central terminal by moving the monitor from a base located on a chest base for LL, RL located on the abdomen and using of a small-plates with electrodes for a two-channel monitor.

FIG. 28B shows sensing with s base inserted into trousers.

FIG. 28C shows various ways of obtaining the LA, RA signal.

FIG. 28D shows a block diagram of the transition of sensing with a Kranz's central terminal to sensing with a Wilson's central terminal.

FIG. 29 shows a block diagram of the communication of the monitor and all cooperating units.

FIG. 30 shows displaying of the curves on the display of the evaluation unit and display unit.

FIG. 31 shows a monitor with electrodes and its use in a holder on a base for application.

FIG. 32A shows a multi-electrode base with an extension on the arm and its use for sensing with a reinforced Kranz's central terminal, as well as a block diagram of the connection of the electrodes to the monitor.

FIG. 32B shows the use of rotatable straps.

FIG. 32C shows sensing with a bridge with electrodes.

FIG. 32D shows the switching between the Kranz's central terminal and the Wilson's central terminal.

FIG. 32E shows an alternative switching between the Kranz's and the Wilson's central terminals.

FIG. 32F shows the use of an abdominal small-plate.

FIG. 32G shows the use of a multi-electrode base.

FIG. 33A shows a monitor attachable on the chest with fixing to the base.

FIG. 33B shows a transition method from a short-term to a long term sensing.

FIG. 33C shows the transition to the long term sensing using a belt shaped base.

FIG. 33D shows the use of an adapter for connecting of a monitor.

FIG. 33E shows the use of a connection system.

FIG. 33F shows the use of an adapter with different connecting elements.

FIG. 34 shows a sensing unit cooperating with a monitor and a connecting field.

FIG. 35 shows a sensing unit with four electrodes.

FIG. 36 shows the cooperation of the monitor located on a wrist with the connection field and the sensor unit in the form of a chest belt.

FIG. 37 shows a mono-channel monitor in a cooperating unit.

FIG. 38 shows a connecting field located in a smart watch.

FIG. 39 shows a monitor located together with a connecting field on a strap cooperating with the chest belt.

FIG. 40 shows the interconnection of the monitor with the connecting field.

FIG. 41 shows the cooperation of the sensing unit with an ECG device according to the prior art.

FIG. 42 shows a monitor with primary by electrodes on its surface.

FIG. 43 shows the interconnection of the monitor, of the interconnecting field and the sensing unit with connecting elements.

FIG. 44 shows the interconnection of the monitor, of the interconnecting field and the sensing unit with connecting elements when the monitor is located in a cooperating unit.

FIG. 45A shows the interconnection of a multi-lead ECG device to a sensing unit.

FIG. 45B shows a standard ECG connection.

FIG. 45C shows the connection of an ECG device to a sensing unit.

FIG. 45D shows connection of a Holter device to a sensing unit.

FIG. 45E shows use of a multi-electrode base for sensing when using a hospital bed monitor.

FIG. 46 shows the switching of the monitor from the sensing mode with, the primary electrodes to the sensing mode with the sensing unit.

### EXAMPLES OF EMBODIMENTS OF THIS INVENTION

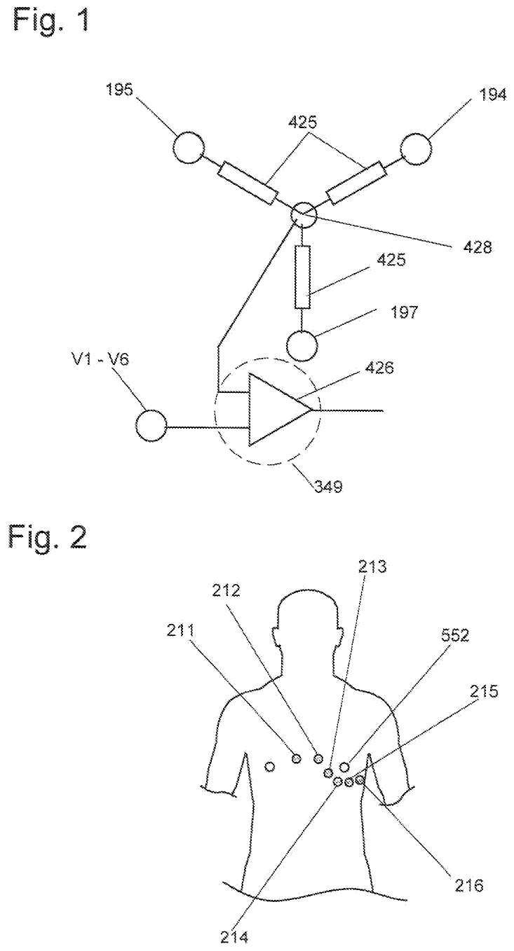

FIG. 1 shows schematically the design of a Wilson's central terminal 428 for sensing of the chest leads V1 to V6.

The Wilson's central terminal 428 we create by applying the potentials sensed by electrodes of the Wilson's central terminal, formed by the electrode 194 RA, 195 LA, and 197 LL from the recommended sensing points, the right arm RA, the left arm LA, and the left leg LL to a common point via resistors 425 of the same value. The potential of the Wilson's central terminal thus formed is applied to one of the inputs of the differential operational amplifier 426 contained in the monitor 349, and is used as reference for sensing and processing the chest lead potentials V1 to V6 on the ECG waveform. The chest lead electrode 211 is successively applied with one electrode to the locations recommended for sensing of leads V1 to V6, and their potentials are applied to the other input of a differential operational amplifier 426. In this way, up to six so-called Wilson's unipolar leads V1 to V6 are obtained sequentially.

FIG. 2 shows the chest locations of the monitored person recommended for placement of electrodes 211 V1 to 216 V6 for sensing of leads V1 to V6. The sites for sensing of leads V1 and V2 are in a level above the nipples, centrally symmetrical around the sternum. The V3 sensing point is closer to the heart at nipple level. The leady V4 to V6 then continue below the left nipple 427 at approximately the same distance from each other in an arc following the bending of the ribs to the left side.

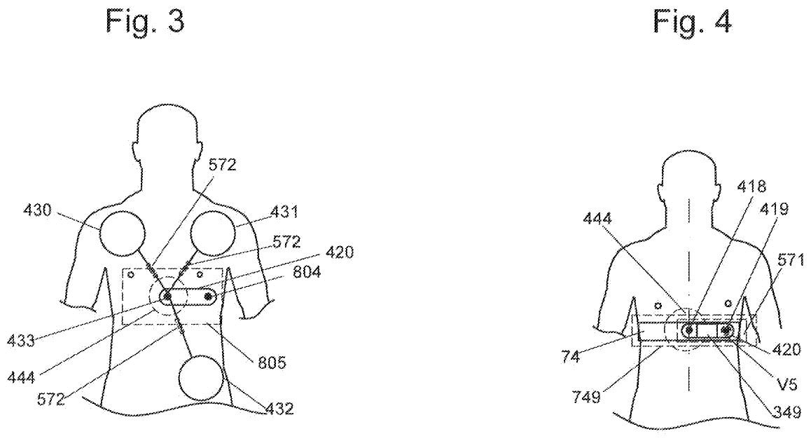

FIG. 3 shows an electrode 143 formed by an electrode for a Kranz's central terminal. The shifted sensing areas of the limbs are marked as the area 430 for the shifted sensing RA′, the area 431 for the shifted sensing LA′, and area 432 for the shifted sensing LL′, from where cardiac signals for the limbs can be sensed instead of conventional areas at the end of the limbs by sufficiently accurate sensing. If we sense signal from the human body from the area in the center between these areas, in the area marked as area 444 of the Kranz's central terminal, we can assume that there is approximately the same resistance between this area 444 and the areas for shifted sensing of RA′, RL′, LL′. Because for formation of a Wilson's central terminal identical resistors are also used from these sensed areas RA′, LA′, LL′, by electrodes located into these areas, it can be assumed, that in the area of a Kranz's central terminal 444 is formed by resistors of the human body, and therefore, the chest leads V1 to V6 can be sensed with reference to it, and it has been confirmed that signal the signal in the area 444 is identical with the signal with a Wilson's central terminal.

The module 805 for sensing of the chest leads by means of a Kranz's central terminal consists of electrodes 804 for sensing od the chest leads located on the chest in the place for sensing selected chest lead and the electrodes 433 for a Kranz's central terminal located in the area 444 of the Kranz's central terminal between the abdominal area and the nipples, wherein, the electrode 804 for the chest leads and the electrode 433 for the Kranz's central terminal are connected to the input of the monitor 349 for sensing of the chest leads Vx.

FIG. 4 shows an example of the location of the base 420 of the universal monitor for sensing of the pseudo-V5.

The Figure shows an example of short-term sensing of the pseudo-chest lead V5 using a universal monitor 349 located on a sensing unit 571, held on the chest by fingers or a chest belt 749, preferably formed by an embracing belt 74, preferably formed by the base 420 with electrodes 143 of the ECG, preferably formed by the electrode 421, applied to the area 444 of the Kranz's central terminal and another electrode 421 applied to the spot recommended for sensing of the lead V5. This makes it possible to sense pseudo-lead V5 for a short time, and similarly also another chest pseudo-leads, for a long-term sensing, the base with the monitor is preferably supplemented with an embracing belt 74 for long term attaching to the chest.

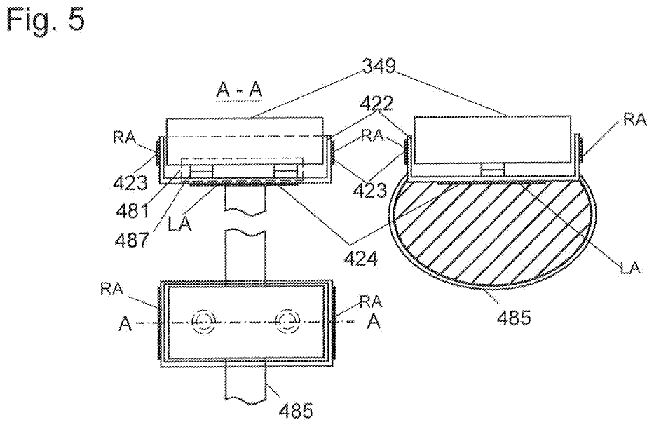

FIG. 5 shows a universal monitor 349 located on a sensing unit 571, preferably formed by a monitor, holder 422 on the wrist, on a wristband of the wristband 485, to which the monitor 349 can be moved from the base 420 or some other senor unit 571. This holder 422 is provided with connecting elements 486, preferably formed by snap fasteners 487. These mediate the electrical interconnection with the ECG electrodes 143, preferably formed by the holder electrode 423 and the lower holder electrode 424, which form the contact surfaces for sensing of cardiac signals of the arms, RA, LA. The electrode 424 of the lower holder is located on the lower part of the wristband 485, so that when so that the wristband of the wristband is applied to the monitored person's arm, it automatically mediates electric contact with the arm, on which the monitored person is wearing the wristband. The contact of the other arm is mediated so that the monitored person places the other hand to the electrodes 423 of the holder, preferably by gripping of the holder in place of the electrodes 423 of opposite sides of the holder 422 by pressing two fingers against each other. At the same time, the person can press the holder, and thus the lower electrode 424 of the holder against the hand, and thus achieve the best possible conductive connection to achieve optimal signal quality. The figure shows location of the wristband 485 on the left arm, so that the lower electrode 424 of the holder provides applying of the signal of the left arm LA and the electrode of the holder 423 of the right arm RA. In case the holder 422 is fixed to the right arm, the opposite is the case.

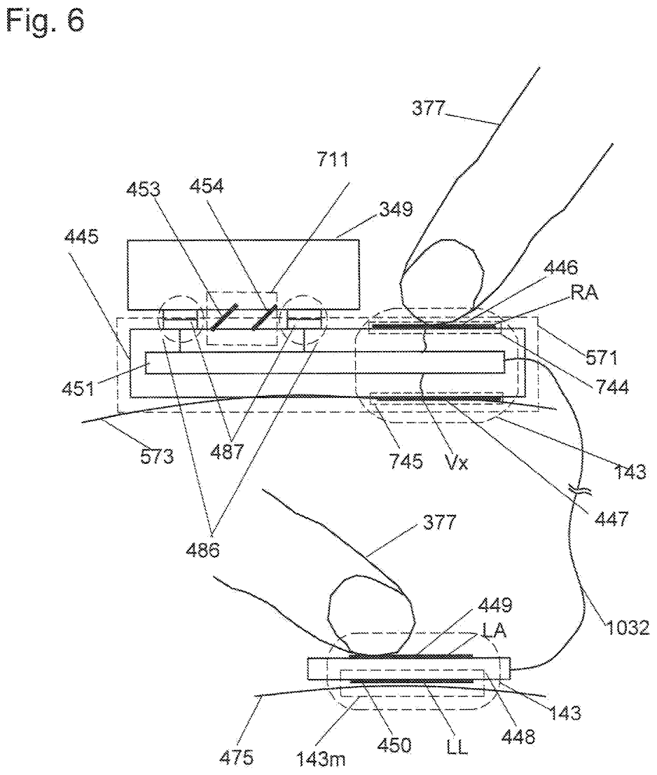

FIG. 6 shows a sensing unit 571, preferably formed by the base 445 of the monitor for sensing of a multi-lead ECG. The monitor base 445 is adapted for attaching of a finger 377, preferably of the right hand, to the ECG electrode 143, preferably formed by an electrode 744 for fingers, in this example formed by an electrode 446 of the holder for a finger, for RA, whereby the holder pressed with its other, the lower ECG electrode 143, preferably formed by a relocateable electrode 143m, preferably formed by electrode 745 attached to the body, in this example formed by an electrode 447 of the holder for sensing of the lower multi-lead ECG “at the location recommended for sensing of leads V1 to V6 on the chest 573, and this successively. This applies also to the finger electrode 446 for a finger placed on the small-plate or the base, as it is shown also in anyone of the following figures, as well as about the electrode of the small-plate 449 of two electrodes for a finger. Preferably, the sensing unit 571 is formed by the small-plate 448 of two electrodes, which comprises an electrode 449 of the small-plate of two electrodes for a finger for LA and the other electrode 450 of the small-plate of two electrodes”, which is applied to the abdomen 475 to sense potential of the left legs LL. The small-plate 450 of the two electrodes “is interconnected by a cable 1032 with the base 445 and the in it comprised electronics board 451 of the holder for a multi-lead ECG, to which it passed on the signals LA, LL. Electronics 746 located on the base 445 allows to sense successively leads V1 to V6, V1R to V6R and V7 to V9. The electrode 446 of the base senses the RA signal. The mentioned electrodes sense all necessary signals LA, RA, LL and chest leads Vx for a 19-lead ECG. The electronics board 451 of the holder for a multi-lead ECG is adapted to connect always two potentials from the electrodes 446, 447, 449 and 450 to the monitor 349, and this with the help of an interconnecting field 711, preferably formed by switches 453 of the leads I, II/Vx and switches 454 of leads LL/RA.

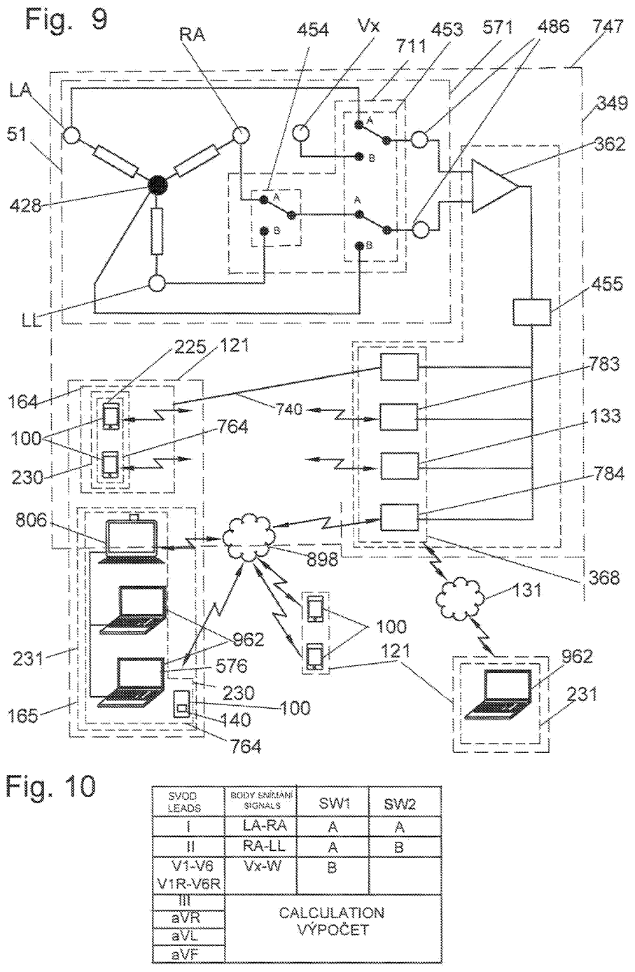

To evaluate the cardiac signals, preferably, a mono-channel monitor, based on the principle shown in the block diagram in FIG. 11 is used Location of the sensing unit 571 on the body by holding it and by holding of the plate is shown in FIG. 9.

Preferably, the monitor 349 is formed by a monitor 770 with electrodes, which is connected by the base 774, as it is illustrated in FIG. 54.

FIG. 7 shows sensing of a multi-lead ECG by electrodes 143 of ECG by attaching of the sensing unit 571 to the body held with fingers, preferably formed by base 445 of the monitor for a multi-lead sensing of ECG, held in right hand and a second sensing unit 571, preferably formed by a small-plate 448 of two electrodes, held by the left hand. In the positions of hands in this Figure, leads I (RA-LA) are sensed, as well as well as the voltage course between the finger of the right hand RA sensed with electrode 446 of the holder for sensing of a multi-lead ECG for a finger and by a finger of the left hand LA, sensed with electrode 449 of the small-plate of two electrodes for a finger, further lead II (RA-LL), as well as the voltage course between a finger of the right hand RA sensed with the electrode 446 and a point on the left side of the abdomen area, that is in the area suitable for placing of the electrode LL, sensed with the other electrode 450 of the small-plate of two electrodes. Leads V1 to V6 are senses by gradual relocation of the monitor base 445 for the multi-lead sensing of ECG so that the electrode 745 for the body is successively applied in places for sensing of the chest leads Vx. By this repositioning, of the monitor base 445 for multi-lead sensing of ECG into positions for sensing of leads V1 to V6 a multi-lead ECG is obtained. By another repositioning of the base 445 it is possible to sense right aide chest leads V1R to V6R and back leads V7 to V9.

The two-electrode small-plate 448, pressed against the body by the pressure of a finger of the left hand is applied alternatively to the left leg, what is practical for persons in sport ware or a skirt. The reference from the right leg RL is not sensed and it is formed artificially by resistors in the monitor 349 or sensing unit 571.

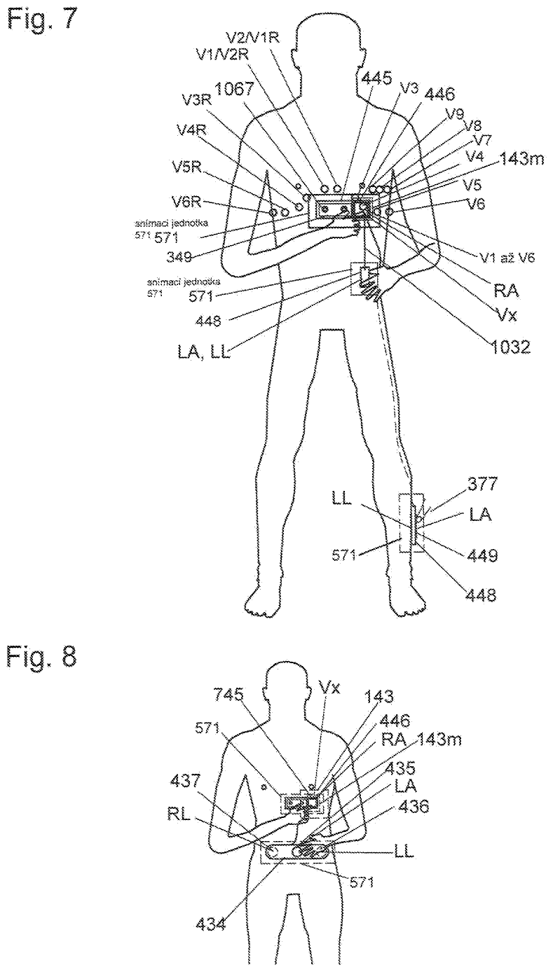

FIG. 8 shows attaching of the sensing unit 571, preferably formed by a plate 434 for leg electrodes with three electrodes, in the abdomen area to obtain a RL reference, which is used alternatively instead of the small-plate 448 of two of electrodes. The plate 434 of leg electrodes with three electrodes preferably comprises on the side allocated for attaching of a finger, electrode 435 of the plate od the leg electrodes with three electrodes for the left hand (LA) and on the side of attaching to the abdomen an electrode 436 of the leg the plate of the leg electrodes with three electrodes for the left leg (LL) and the electrode 437 of the plate of the leg electrodes with three electrodes for the right leg (RL). In comparison to the plate 448, this electrode is extra in comparison to the plate 448 and serves for obtaining of the lead RL, which serves as a reference.

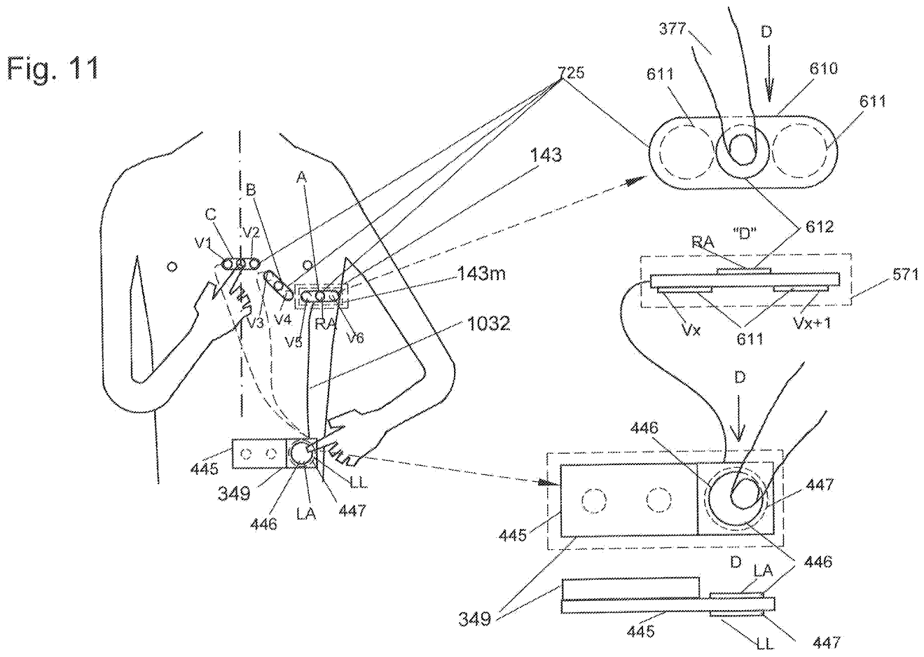

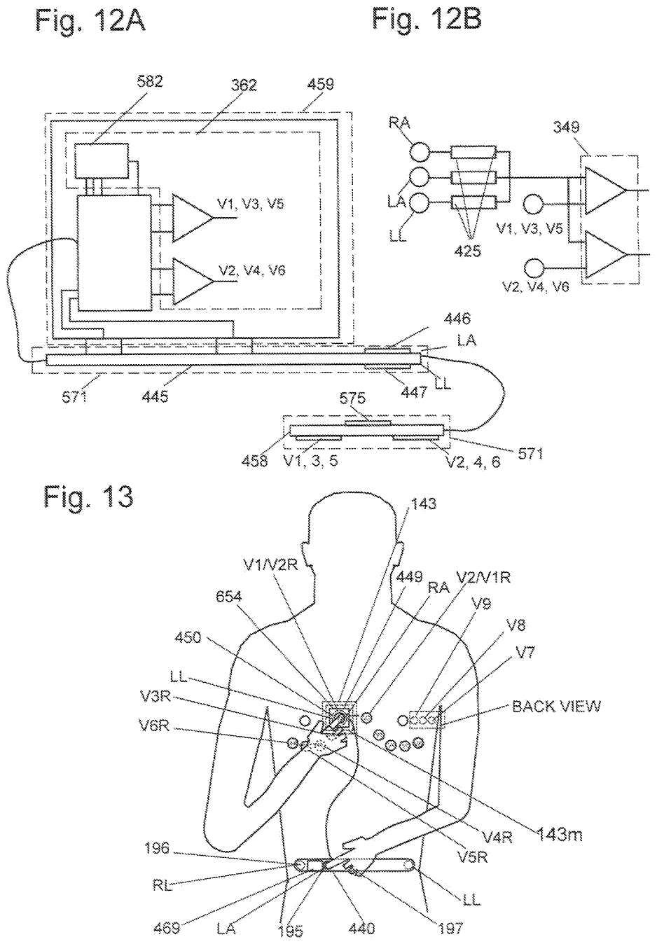

FIG. 9 shows a block diagram of an interconnecting field 711 of a handheld monitor for sensing of leads I, II and V1 to V6 by a sensing unit 571, which unit is formed preferably by a base 445, see FIG. 8, and transmitting by the monitor 349 of the processed signals to the cooperating units 121 and to the selected participants. By using the small-plates 448 of two electrodes for LA and LL or of the plate 434 of the leg electrodes, see FIG. 7, with three electrodes also for the RL, and by the successive attaching of the base 445 of the monitor for a multi-lead sensing of the ECG “for RA and V1 to V6, and V1R to V6R and V7 to V9, as it is shown in FIG. 12 and by switching over the switch 453 of leads I, II/Vx and 454 LL/RA the signals LA, RA and LL are connected to the Front-end 362, whereby sensing of leads I, II is reached. Another 4 leads III, aVR, aVF, aVL for obtaining of up to 19-lead ECG are calculated from the leads I, II, wherein, for this calculation pulses of the lead I and pulses of the lead II having the same length R-R are chosen. The control unit 455 can process and transmit a complete ECG via the transmission module 459 to the cooperating unit 121, and to the near cooperating unit 164 and/or to remote cooperating unit 165, for example for medical personnel, consisting of desktop units 231, preferably formed by server 806, PC 962, and handheld units, preferably formed by a mobile phone 100. For connection to the cooperating unit 164, the transmission module 459 comprises preferably the a WiFi unit 783, a Bluetooth module 133, a Zigbee module, and for connection to the cooperating unit 165 a module 784 for transmission via a Mobile operator's network 898.

Preferably, the monitor 349 is formed by a monitor 770 provided with electrodes, connected by an adapter of the base 777.

FIG. 10 shows a table of positions of switches of the base 445 of the monitor for a multi-lead sensing of ECG for sensing of individual leads. The first column indicates the names of the leads to be scanned, the second one indicates the recommended points for sensing of the leads, between which it is scanned, the third one specifies the positions of the switches 453 of the leads I, II/Vx for sensing of the required lead and the fourth one indicates the positions of switches 454 for the leads LL/RA for sensing of the desired lead. The first column shows the names of the leads to be scanned, the second indicates the recommended points for sensing these leads, between which it is scanned, the third column shows the positions of the 453 lead I, II/Vx switch for sensing the desired lead, and the fourth column the position of the LL/RA lead switch 454 for sensing the desired lead. Leads III, aVR, aVL and aVF are calculated. When sensing the leads V1 to V6, the switch 454 of leads LL/RA is not used, and the Vx electrode, preferably formed by the electrode 447 of the holder for sensing of the lower multi-lead ECG, for sensing of V1 to V6 is moved successively to the points recommended for sensing of V1 to V6. The reference from the RL is obtained by shifting of the plate 434, by which the RL is sensed, or when using the small-plate 448 the RT reference is created artificially by resistors in the monitor 349 or in the sensing unit 572.

FIG. 11 shows location of the monitor 349, preferably formed by a two-channel monitor 459 on a sensing unit 571, preferably formed by a base 445 of the monitor for the multi-lead sensing and its use. The sensing unit 571, preferably formed by the base 445, is provided with two ECG electrodes 143, preferably formed by the electrode 447 of the base for sensing of a lower multi-lead ECG for sensing of LL and an electrode 446 of the base for sensing of multi-lead ECG for finger, for sensing of the lead LA. Also the small-plate 610 of three electrodes with the electrode 612 of the small-plate of three electrodes for finger “for sensing RA and with two electrodes 611 of the small-plate of three lower electrodes for sensing Vx and Vx+1 is connected to the monitor by cable.

The signals from electrodes 612 (RA), 446 (LA) and 447 (LL) are used for formation of the potential of the Wilson's central terminal, against which the chest leads are sensed, that is the signals Vx, which are sensed by two electrodes 447 on the lower side of the base of the small-plate 610. The “D” view shows a side view.

The small-plate 610 of three electrodes is held by a finger of the right hand in the place of the electrodes 612 for sensing of the RA, wherein, the electrodes 611 sense the leads V5, V6 in the position A, the leads V3, V4 in the position B, and the leads V1 and V2 in the position C. The base 445, on which the monitor 349 is connected, is pressed by a finger in the place of the electrode 446 for the LA electrode 447 in the abdominal area for the LL.

Further, FIG. 11 shows successive attaching of the base 464 of a two-channel monitor for finger with the monitor 459 placed in places on the chest that are recommended for sensing of leads V1 to V6, and to always of two of them, that is the first sensing V1 and V2, the other V3 and V4, the third V5 and V6. Simultaneously, always together with the pair of leads Vx and the potential of the right arm RA, also the potentials LL and LA are sensed with the small-plate 448 of two electrodes.

FIG. 12A shows a block diagram of connecting of a two-channel monitor 459 cooperating with the base 445 for multi-lead sensing, applied to the abdomen, with a left hand finger and a three-electrode chest plate 458, applied to the chest for the arrangement shown in FIG. 11. With this configuration, leads I, II are sensed at first, and then always pairs of leads Vx and Vx+1. Since these leads are sensed in reference to the Wilson's central terminal, potentials from the respective electrodes, applied to the recommended places for sensing are sensed at the same time to obtain its potential, and they are the an electrode of the chest plate 575 of three electrodes, chest, for a finger “for RA, the electrode 446 for LA and the electrode 447 for LL. Preferably, all signals are fed via a switching field 574, to the input circuits Front-end 362 in the monitor 349, where in the circuit 582 of the Wilson's reference a reference Wilson's central terminal is formed from the potentials LA, RA and LL, and against it, the leads V1 to V6 are evaluated.

FIG. 12B shows circuits with formation of the Wilson's central terminal from the resistor 425 off the monitor 349.

FIG. 13 shows sensing of a multi-channel ECG using a three-channel monitor located on the abdominal base 440 of the monitor, and electrode small-plates 654 on a cable. The abdominal base 440 of the monitor is similar to the plate 434 of leg electrodes with three electrodes “, but additionally, it is adapted for mounting and connecting of the three-channel monitor 469, and allows interconnection by a cable with further sensing units, i.e. the bases or plates of electrodes.

In the embodiment shown in the figure, the monitored person holds the “base 440 of the abdominal monitor” with his left hand, preferably with the index finger, what makes possible to sense potentials from the RL, LL and LA sites.

With the right hand, the monitored person places the plate 448 of two electrodes “to spots for sensing of the chest leads, which plate comprises second electrode 450 of the plate of two electrodes on the side attached to the chest, which senses appropriate V lead, and one electrode on the side turned away from the chest, what is the electrode 449 of the plate of two electrodes for finger”, which senses RA. The sensing unit 571, so equipped with a three-channel monitor, is adapted to sense simultaneously the leads I, II and one chest lead Vx, wherein, for displaying 4 leads are calculated, i.e. the II, aVF, aVL, and aVR. Another leads Vx are obtained by gradual relocation of the plate 448 of two electrodes into the points recommended for sensing of individual leads V1 to V6 for sensing of a 12-lead ECG. The reference RL is used for reference.

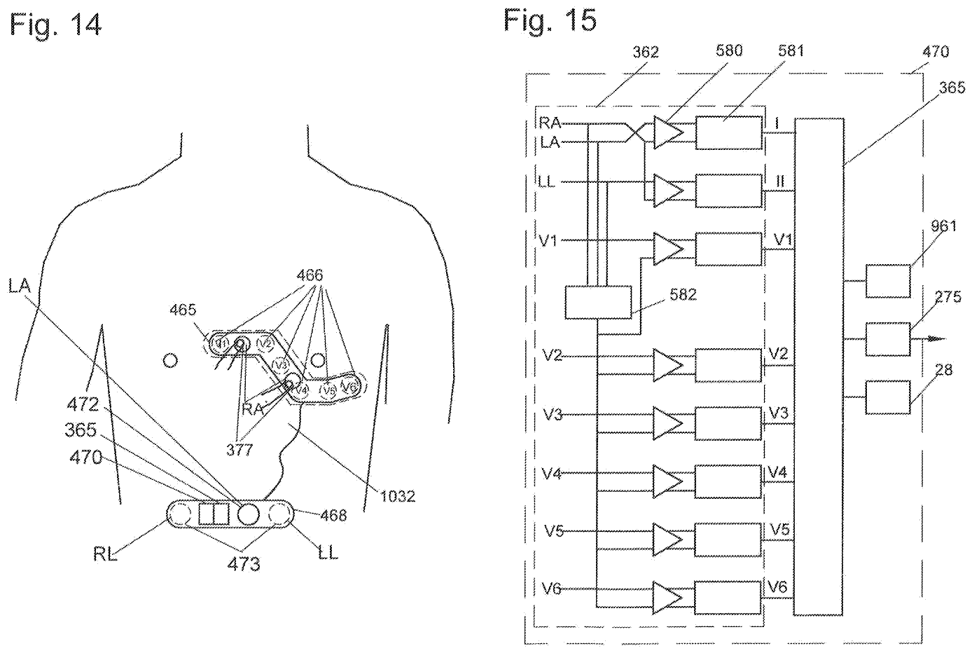

FIG. 14 shows the sensing of a 12-lead ECG at one time with a sensing unit 571, preferably formed by a chest electrodes plate 465 “. This plate is shaped into a curve to that the six ECG electrodes 143, preferably formed by the chest plate 466 electrodes for sensing of chest leads, located on the side to be applied to chest, touch the chest in places recommended for sensing of leads V1 to V6. Further, on the top side, the plate 466 is provided with ECG electrodes 143, preferably formed by two interconnected electrodes 474 for sensing of the signal of the right arm RA from fingers 377, and this in places suitable for pressing of the plate 466 with two fingers to the chest. The plate 465 is interconnected by a cable 1032 with the sensing unit 571, preferably formed by the multi-channel base 468, which base is provided with a monitor 349 provided with the number of channels necessary for simultaneous sensing of the planned number of ECG leads. The multi-channel base 468 is pressed with fingers of the right hand against the abdomen area. On the abdominal side, the base has two ECG electrodes 143, preferably formed by bottom electrodes 473 of the multi-channel base, for sensing at the LL and RL points. In case the base 473 is equipped with an eight-channel monitor 470, this gives the required number of channels for leads I, II, V1 to V6 that are sensed, and the other leads II, aVL, aVR and a VF are calculated.

FIG. 15 shows a block circuit of the monitor 349, preferably formed by an eight-channel monitor 470. Compared to the three-channel monitor 469, this monitor is extended by 5 channels, which allow simultaneous scanning of all left-chest leads, and of leads I and II. Processing of the required 8 channels is provided by the control unit 365 for a twelve-lead ECG for storage in memory 961, and/or displaying on the display 28, or transmitting via the communication unit 275 to the cooperating units 121, and/or to selected participants.

Preferably, the memory 961 is extractable. Preferably, it is formed by a SD card 959, which card allows relocation to the cooperating data transfer units 121.

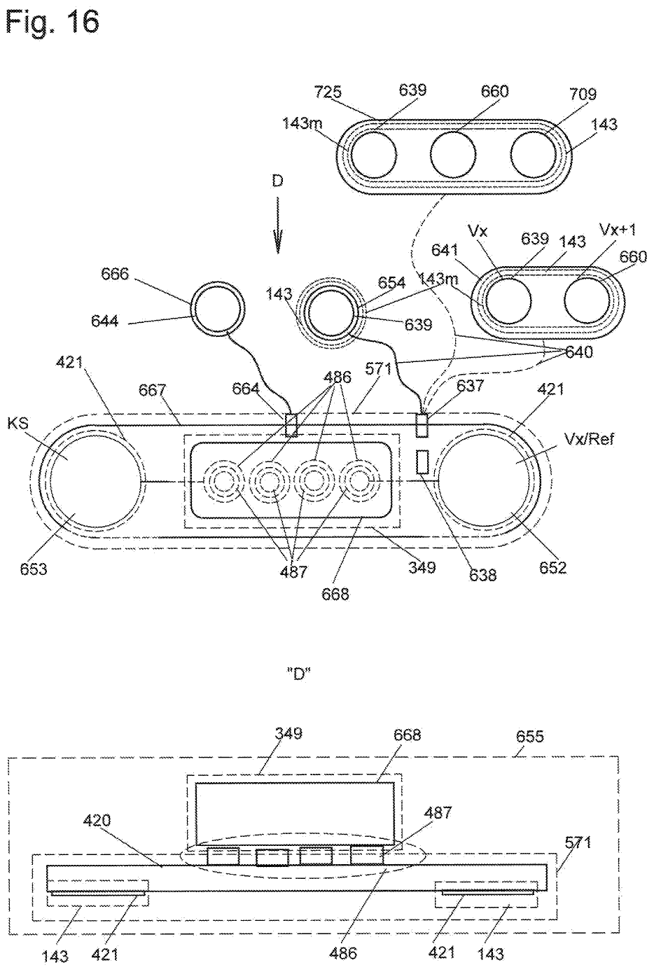

FIG. 16A shows a sensing module 655 consisting of a monitor 349 and a sensing unit 571 for sensing of chest leads V1 to V6, V1R to V6R and V7 to V9 with the Kranz's central terminal, preferably using reference electrodes 644, and further, the Figure k shows the base 667 with a reference of a universal monitor 349, preferably a mono-channel one, which is provided with electrodes 421, preferably with one electrode 652 formed for leads Vx/REF for sensing of chest of leads Vx, which is switchable with a switch 638 Vx/REF “to reference REF, and simultaneously disconnectable from the input of the operational amplifier 406, taken out to the connector 637 and is connected by a cable 640 to the electrode 639, which electrode is connected for sensing of Vx in case, the electrode 652 is connected to Ref. Alternatively, the cable 640 connects electrodes 639 for Vx and 660 for Vx+1 on the small-plate 641 of electrodes for the two-channel monitor for sensing, preferably optionally of all chest leads, relocation. Preferably, the other electrode 421 is formed by the electrode 653 for KS/LL, i.e. the electrode for sensing of potential of the Kranz's central terminal, alternatively for signal of the left leg LL. The electrodes are formed by round or elongated large-area grouped electrodes 471 or by electrodes with smaller size for sensing of individual leads.

Preferably, the base 667 serves for provision of a mono-channel monitor or a dual-channel one.

With a connector 637 for Vx either the small-plate 654 electrodes on cable, are connected or the small-plate 641 of electrodes for the dual-channel monitor are connected. Alternatively the small-plate 725 of three chest electrodes is connectible for cooperation with the three-channel monitor.

With the connector 664 of reference the electrode 644 of reference is connectible on the small-plate 666 for securing of reference instead of an artificial reference, which is disconnected by the switch 643 of the reference, preferably automatically when the pin connector 664 of the reference is inserted.

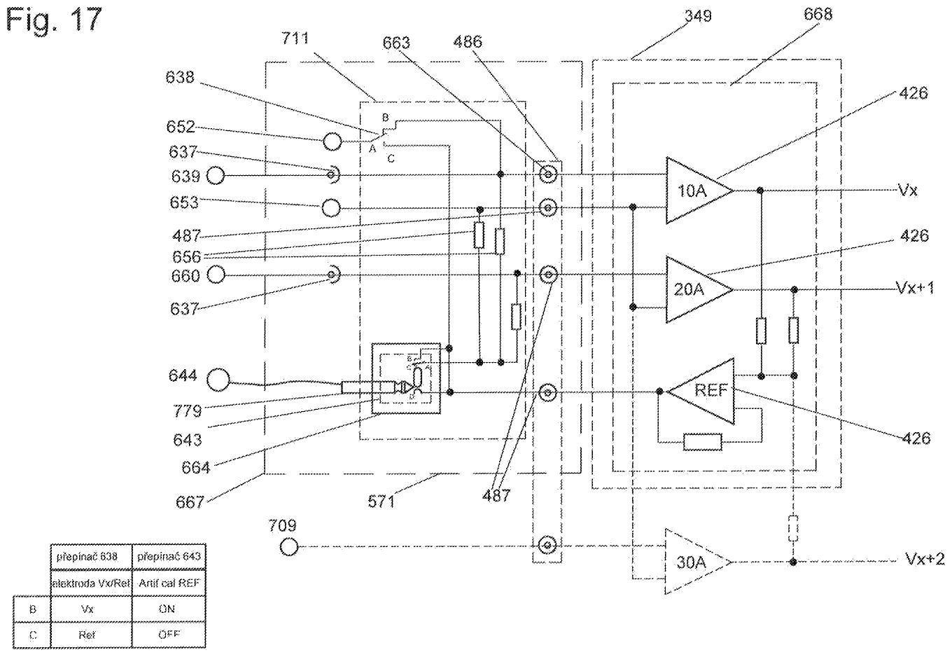

Thus the base 667 comprises a switch 643 of the reference, and preferably a switch 638 Vx/REF for different sensing possibilities, so as it is shown in FIGS. 16B to 16C and in Table in FIG. 17.

The Detail “D” shows configuration of the sensing module 655, which is formed by the sensing unit 571, and by the heart rate monitor 349, which is fastened to the sensing unit, and together with it, it also conductively connected by means of connecting elements 487.

FIG. 16B shows sensing of chest leads against the Kranz's central terminal using the base 667. The electrode 653 is applied to the area 444 of the Kranz's central terminal and the electrode 652 Vx/REF is attached to places recommended for sensing of chest leads Vx, in this Figure it is specifically the right-side chest lead V5R. At the same time, the reference electrode 644 on cable is utilized, which is clamped preferably in the right armpit.

FIG. 16C shows the use of the base 667 for sensing of chest leads with the help of the small-plates 654, wherein, the abdomen area is used for reference by attaching the electrodes 652 Vx/REF, which are switched by the switch 638 Vx/REF to the position C ref. The switch 643 in FIG. 17 is in the position C, when the feedback resistors 425 are not used and the reference signal supply provides the electrode 652, which is set to the reference mode of this electrode so as it shows the block diagram in FIG. 17. With the small-plate 654 of the electrode to cable, connected to the monitor by the connector 637 of the electrode Vx, all chest leads shown in FIG. 16D are sensed successively.

FIG. 16D shows use of the base 420 for sensing of leads Vx, always two at a time, using a two-channel monitor 459 and small-plates 641 of electrodes for the two-channel monitor.

FIG. 17 shows a block diagram of connections of the input circuits of the monitor 349 from FIG. 16, preferably formed by monitor 668 with a taken out reference.

The sensing units 571, are formed preferably by the base 667, the monitor 668, which is connected mechanically and electrically to the base 667 by four connecting elements 486 preferably formed by snap fasteners 487. The switch 638 Vx/REF connects the electrode 652 from Vx to Ref. The reference electrode 644 is connected through the reference connector 664, which during inserting of the connector 779 of the pin connector preferably switches automatically the switch 643, which disconnects the pseudo-reference.

Electrode 639 for Vx on the small plate 654 of the electrode on the cable and the electrode 660 are connected through the connector 637. This allows to sensing of one chest lead of the ECG electrode 652 or electrode 639 on the cable 640 or of two ECG leads by electrodes 639 and 660 to cable 640. It is sensed either with the artificial reference formed by means of resistors 425 or with reference signal injected in the right armpit of the monitored person by electrode 644 or electrode 652 switched with switch 638 into the reference mode.

The table specifies positions of switches 638 and 634 for sensing of Vx or use of the artificial reference. The dashed line shows the third operational amplifier 626 for the electrode 709 for Vx+2, which electrode is added preferably to the electrodes served by the first two operational amplifiers 626 for sensing of three Vx leads at once.

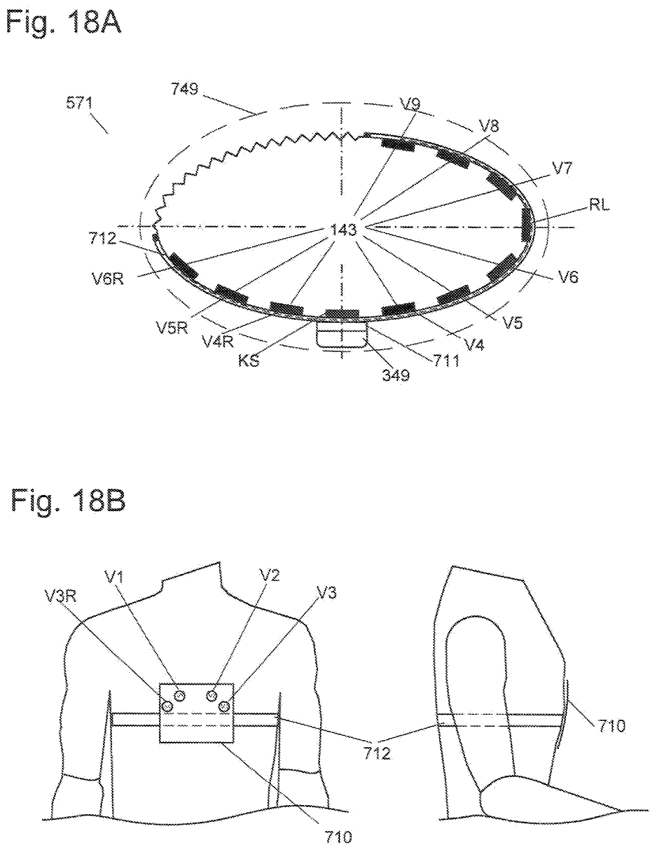

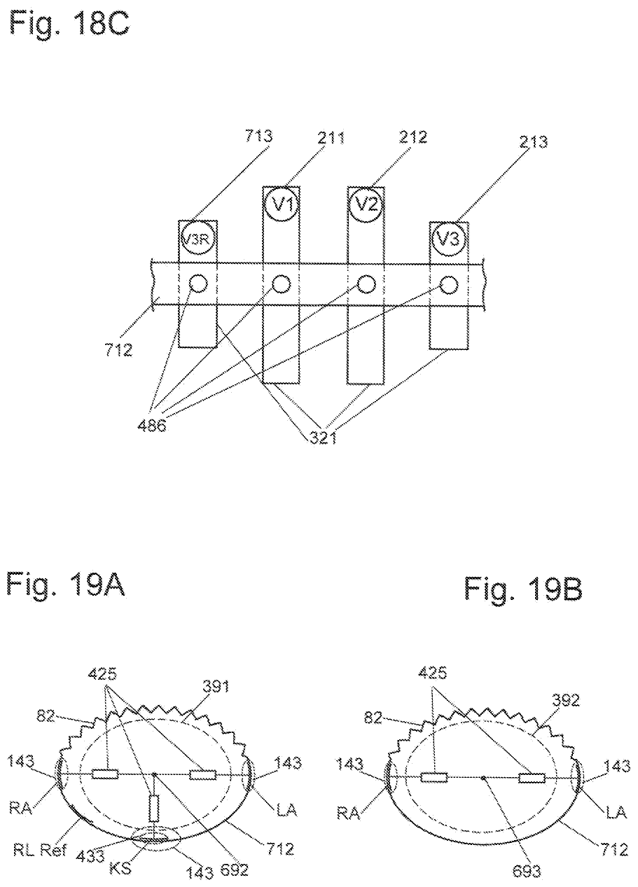

FIG. 18A shows the principle of the sensing unit 571, preferably in the form of a chest belt, for sensing ECG of two, three, or up to dine channel monitor 349, or in case of a greater number of electrodes 143, also with a multi-channel monitor 349, using the Kranz's central terminal KS. Preferably, the sensing unit is formed by the complete base 712, which is applied to the chest, or after clipping of the resilient part 82, encloses the chest. Base is provided with electrodes 143 so that they rest on the chest and on the back at the locations recommended for sensing of V6R, V5R, V4R, V4, V5, V6, V7, V8, and V9. Further, there is one electrode 143 for KS in the middle of the chest and one electrode 143 for RL on the left side. Thus, the sensing unit 571 can sense all potentials in the recommended sensing locations that are in one line. The electrodes 143 are led to the interconnecting field 711. The sensing with the sensing unit 571, preferably formed by the base 712, is designed to be the best possible one, using only electrodes located on the sensing unit in the form of a belt. Therefore, the sensing of leads I, II and also of leads V1/V2R, V2/V1R, V3, V3R is missing. If it is shown that these leads are needed, an expansion plate 710 is clipped to the base 712 to allow sensing of the leads V1/V2R, V2/V1R, V3, V3R, as well as to connect the electrodes to the monitor inputs. Alternatively, separate electrode segments 321 are used to sense these leads, which are resilient and connected by connecting elements 486. The monitor 349, the plate 710 and the electrodes 143 are connected to a connecting field 711 which field allows the respective lead to be applied to the respective monitor channel via the connecting field 711.

When using a monitor 349 with fewer channels than what is required for the number of electrodes used, it is sensed successively with the number of electrodes that can be connected to the monitor at once, and further electrodes for subsequent sensing are connected to the monitor through the connecting field 711 subsequently.

FIG. 18B shows use of an expanding plate 710 in cooperation a complete base 712. The plate 710 comprises four electrodes, which after connecting of the expanding plate 710 to the complete base 712, rest on places recommended for sensing 211 for V1/V2R, 212, for V2/V1R, 213, for V3, and 713 for V3R. The plate 710 is fixed on the base 712 centrally, so that it the exceeds and preferably, it is slightly bent to press the electrodes 143 against the chest, as it is seen in detail D. The plate 710 is usable for sensing in all types of sensing units 571 having the shape of a chest ho belt.

FIG. 18C shows use of electrode segments 321 to supplement sensing also of the leads V1/V2R, V2/V1R, V3, and V3R. The electrodes 143, preferably formed by the electrodes 211 for V1/V2R, 212 for V2/V1R, 213 for V3 and 713 for V3R are fixed with a connecting element 486 to the complete base 712 and they are connected to the connecting field 711. For clarity reasons the connecting field 711 and the monitor 349 are not shown.

This solution makes it possible to sense signals at points remote from the longitudinal axis of the base 712 and at the same time, since the electrode segments are resilient and suitably shaped, pressure of the electrodes 143 on the skin is ensured.

The segments 321 are applicable to all types of sensing units 571 in the shape of chest belt.

FIG. 19A shows a method of obtaining a reinforced Kranz's central terminal 692 for more accurate sensing with circuits for the strengthened Kranz's central terminal, preferably formed by three resistors 425 connected between electrodes sensing potentially RA, LA and KS, to obtain the center of these signals., Individual chest leads are sensed against this center. According to the used sensing means the resistors 425 are located in the base 712, in the connecting field 711, or in the monitor 349.

FIG. 19B shows formation of a pseudo-Kranz's central terminal by connecting the potentials RA and LA through resistors 425 to a single point, KS.

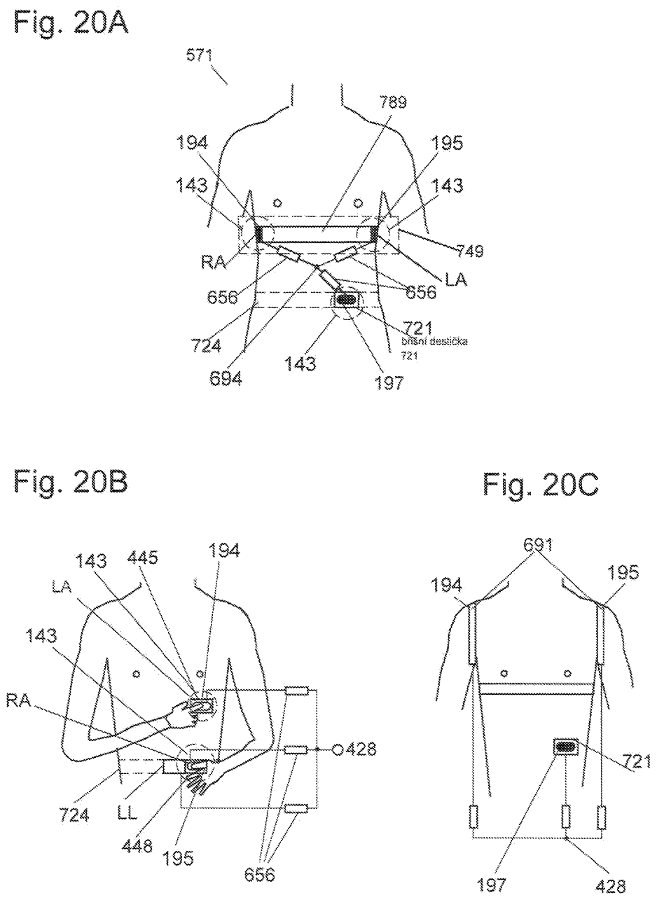

FIG. 20A shows formation of a pseudo-Wilson's central terminal 694 from RA, LA potentials obtained by electrodes 143, preferably formed by electrodes 164 RA and 195 LA of the base 712, located on sides of the monitored person, and potential LL obtained preferably by means of electrodes 143, preferably formed by the electrode 197 LL on the abdominal small plate 721, led by resistors 425 to one point. The pseudo-Wilson's central terminal is used preferably so that for all specified sensing against the Kranz's central terminal, the potential KS is substituted by the potential of the pseudo-Wilson's central terminal. By this, it is possible to obtain signal of improved quality. The advantage of the Kranz's central terminal over the pseudo-Wilson's central terminal is that all electrodes 143 are located in one level preferably on the bases 789 in the shape of a belt. The advantage of the pseudo-Wilson's central terminal over the Wilson's central terminal is that only one electrode 197 for LL is located off the base 789 in the form of a belt. Preferably, the base 789 in the form of a belt is formed at least by one of the following: elongated base 624, base 620 for RL, simplified base 720, and multi-electrode base 630.

FIG. 20B shows an example of sensing with a full-fledged Wilson's central terminal 428 from the left arm and the right arm LA, RA and the left leg LL. In this embodiment of the FIG. 8 the monitored person holds the base 445 of the monitor for multi-lead sensing of ECG by pressing the electrodes 143 for the right arm RA with the right hand, at best pressed to the chest. With the left hand the person holds the small-plate 448 of two electrodes by pressing the electrodes 143 for the left hand LA, whereby pressing of two electrodes RL, LL on the abdomen is reached.

FIG. 20C shows another example of obtaining the Wilson's central terminal 428 from RA, LA using a shoulder strap 691 with an electrode and the electrodes 197 LL on the abdominal small-plate 721.

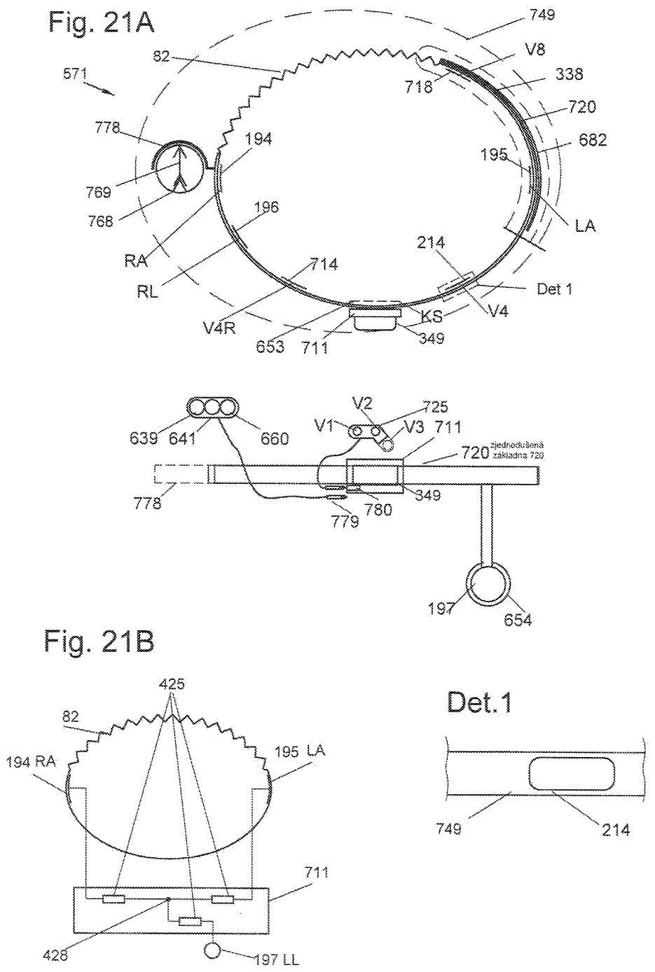

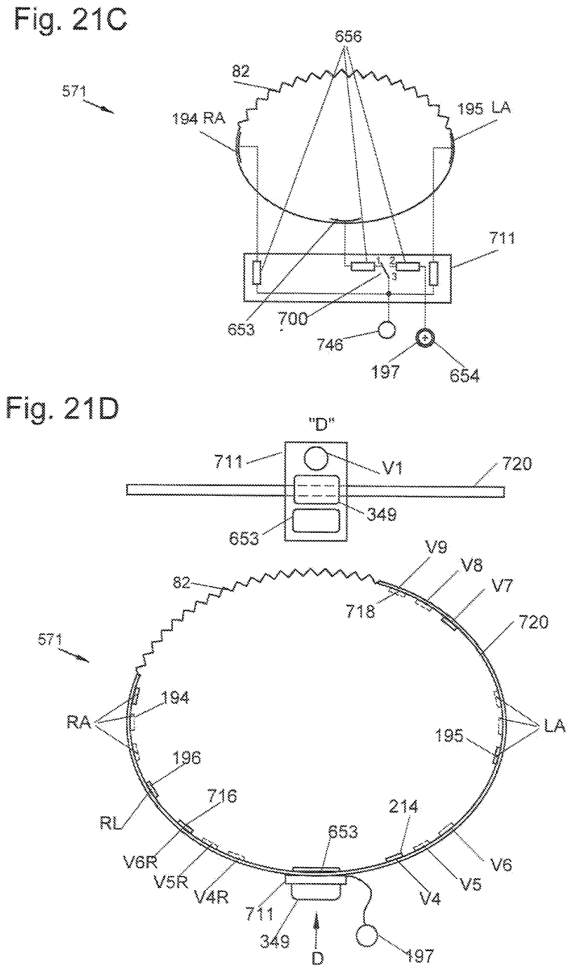

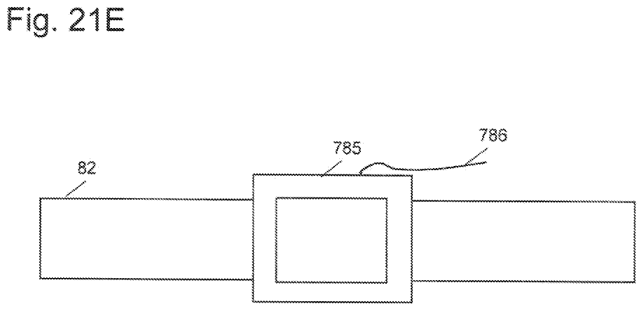

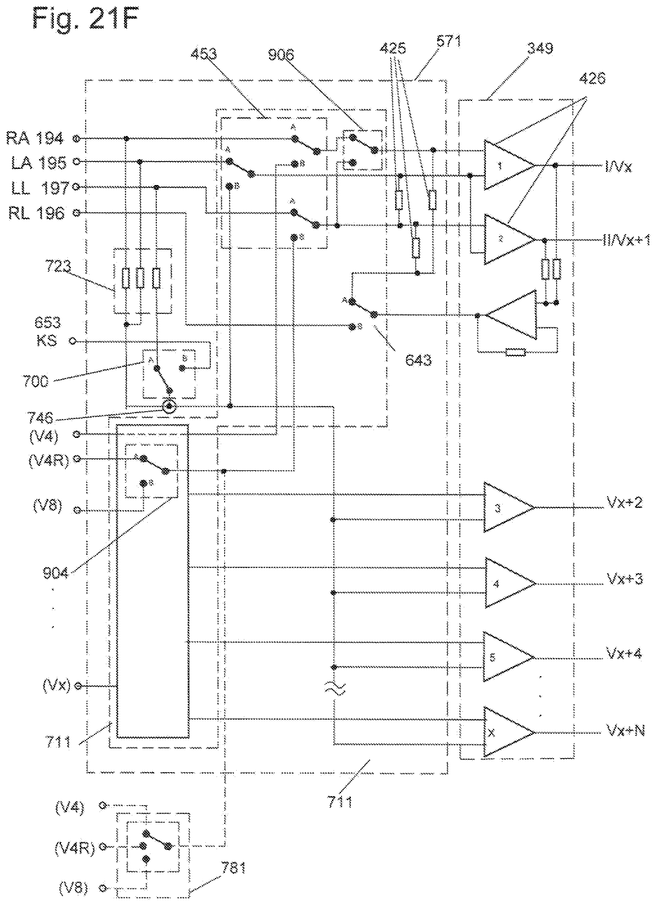

FIG. 21A shows chest belt 749, preferably formed by a simplified base 720, which is allocated to be used for sensing of the optional chest leads, in this example of leads V4R, V4, and V8 against the pseudo-Wilson's central terminal by means of electrodes 714 for V4R, 214 for V4, and 718 for V8R. These leads were chosen, because they are significant for capturing of the elevation ST of the segment. IN order to obtain a stronger cardiac signal for higher quality of displaying and during the physical movement of the monitored person, the electrodes 714, 214, 718 are made with large area as shows in Det. 1, and the area is at least 20×30 mm or more up to 20×100 mm.

The chest belt 749, preferably formed by a base 720, or also any other base, for example the prolonged base 624, it is provided by an extending part 682 of the base, preferably formed by a strut 338, which strut allows easy inserting of the base with right hand under the garment, for example a shirt, without undressing, as it does not bend and holds the direction of insertion. Preferably, the chest belt 749 is provided with an extension 778 for the arm, which extension allows to the monitored person, to exert higher pressure on the base to the body through the garment, and so to improve contact of electrodes with the skin.

After inserting of the extended part 682, the monitored person, at first thoroughly rests against the backrest of the chair, whereby, pressure is exerted on the base in the place of the back electrodes 718 for V8 and prevents sliding away of the prolonged part 682 away from the back. Subsequently, by moving of the right arm backwards in the direction of the arrow 747, the hand pressure acts on the arm extension 778, whereby additional pressure is applied on the base to the body and thus on all electrodes on the base.

Alternatively, it is possible, instead of the extension 778, to switch, for pressing of the chest belt 749 to the body, the elastic part 82 of the belt. When the elongate portion 82 is long enough, it is possible to insert the chest belt 749 around the back with the right hand and with the left hand, which is under the shirt, from the end of the elongate portion 682 grasping the applied elastic portion 82 and fix it to the beginning of the belt, and after turning it back into the operational position to reach putting on of the belt without taking off the shirt.

In a preferred embodiment, the connecting field 711 comprises also a ferrule connector 780 u with a switch. After connecting of the pin connector 779 to the ferrule connector 780, the interconnecting field 711 is switched over from electrodes on the chest belt 749 to the electrodes on the small-plate 654 or 725, for example the electrodes 714 for VAR and the electrodes 718 for V8 are switched over to the electrode 639 for Vx and to the electrode 660 for Vx+1, which electrodes allow, after attaching of the small-plates 654 or 725, to sense the leads V1, V2, V3, V3R and other selected chest leads. The small-plates 654 or 725 for sensing of the chest leads are connected to the base with the pin connector 779.

In an alternative solution, the small-plates 654 or 725 are connected by a connector and the switching over of the electrodes will be carried out by a switch or through the connecting field 711.

FIG. 21B shows a pseudo-Wilson's central terminal, which is formed from signals RA, LA connected via resistors 425, which signals are sensed by electrodes 194 and 195, which are located on the chest belt 749, preferably formed by the base 720 so that they abut on chest sides of the monitored person, and from the signal LL, which is applied from the electrode 197 for LL on a cable, which electrode is placed in the left abdominal area and it is held by hand, or preferably, it is inserted under the trousers'waist belt or it is fixed by the abdominal belt 724. The electrode 197 for LL is connected to the interconnecting field 711, whit which the selected electrodes are connected to the monitor 349 as needed. The electrode 196 for RL located on the chest belt 749, preferably formed by the simplified base 720, is utilized preferably for reference. Alternatively the pseudo-Wilson's central terminal is switchable by the switch 700 KS/WS, which switch is shown in FIG. 19C.

FIG. 21C shows in a block diagram the principle of switching between sensing using a Wilson's central terminal and sensing using a Kranz's central terminal. The switch 700 KS/WS switches from position 1 to position 3, whereby the signal is switched from the electrode 653 for KS, conducted via the resistor 425 to the strengthened Kranz's central terminal obtained from other signals LA, RA with resistors 425, to the electrode 197 for LL, whereby in the point 727 of the KS/WS terminals it is switched over from the strengthened KS terminal to the WS terminal. By switching over of the switch 700 KS/WS into the position 2, the electrode 653 for KS and also the electrode 197 for LL are disconnected and on the clip 746 the signal pseudo-KS will be present, which signal corresponds to the signal on the clip 693, which clip was described with regard to FIG. 17B, The switching over can be implemented in the connection field 711.

FIG. 21D shows use of a sensing unit 571, preferably formed by a chest belt 749, preferably formed by a simplified base 720 for sensing of additional chest leads by slight rotating of the base 720. In this embodiment, the base is provided with suitable electrodes for slight rotation, in this example, instead of the electrode 714 for V4R it is provided with an electrode 716 for V6R, and instead of the electrode 718 for V8, with an electrode 717 for V7.

In the basic position of the base 720, the leads V6R, V4 and V7 are sensed. After the first slight turning of the base 720 to the left, relative to the body of the monitored person, by approximately 3 to 4 cm, the leads VSR, V5, and V8 are measured, and after the second slight turning of another approximately 3 to 4 cm, the leads V4R, V6 and V9 are measured.

Although the electrodes 194 for RA and 195 for LA move along the sides of the monitored person, their signals are still equally useful for obtaining the RA and LA signals with sufficient accuracy.

Preferably, the electrode 653 is covered by a transverse strip 787 fitted with two electrodes, one for Vx and one for KS/LL. The transverse strap 787 snapped to the base, and it is connected with the connecting field 711. The electrode Vx senses the lead V1 (V2R) in the basic position. The electrode KS/LL is used for LL. After the first slight turning to the left from the point of view of the monitored person, the Vx electrode senses the lead V2 (V1R), and after the second one the lead V3. This significantly expands the sensing options. Optionally, all types of KS or WS, shown in FIGS. 19A, 19B, can be used.

FIG. 21E shows a pull-on electrode 786. These electrodes are pulled on an elastic part 82 of the belt, which part is pressed to the chest of the monitored person. An advantage of such electrode is the possibility to place is as accurately as possible into the position recommended for sensing of a specific chest lead. The contact area of the electrode put out via the output 786 of the electrode, which is connected to the connecting field 711.

FIG. 21F shows a connection block diagram of the monitor 349, including the interconnecting field 711, which interconnects the electrodes 143 to the input circuits of the monitor 349 for various sensing unit 571. Its function is apparent from the example for the simplified base 720 and the two-channel monitor.

To the inputs of the operational amplifiers 426 are brought alternatively the signals RA, LA, LL for sensing of leads I and II and for calculation of the leads III, aVR, aVL and aVF by the switch 722 in the position A and after switching of the switch 722 to the position B the signals Vx, in this case, by the switch 904, the V4R/V8 is switched to sensing of V8.

When using a three-channel monitor, the V4, V4R and V8 are sensed simultaneously. For a mono-channel monitor, se for a successive sensing of V4, V4R and V8 is used the switch 781 V4/V4R/V8 shown as a connecting field 902 for Vx.

This connection makes possible to work with the reference RL, supplied to the body of the monitored person in the position B of the switch 643 of the reference or with an artificial reference, supplied via the resistors 425 inputs of said operational amplifiers in the position A and the switches 643 of reference. The signals Vx are measured against the Wilson's central terminal, which is formed with circuit 723 of the Wilson's central terminal in the position A and the switch 700 KS/WS. In the position B, the electrode 653 for KS/LL is connected for sensing with KS.

When using a five-channel monitor 349 with the help of a simplified base 720 from FIG. 21A, it is possible to sense in the position A and the switch 722 (I, II/Vx, Vx+1) leads I, and II, and further simultaneously always the triads of the chest leads, always against the Wilson's central terminal, V6R, V4, V7, further VSR, V5, V8 and further the V4R, V6 and V9 with the help of operational amplifiers 3 to 5. In case of switching the switch 722 to the position B, simultaneously, it is possible to sense 5 selected chest leads simultaneously.

The signals from the individual electrodes are brought to the connecting field 711, which field makes possible to connect the any input to any output, and thus to a channel of the monitor. The base 720 according to the example is designed to sense five leads at once, and therefore, to use a five-channel monitor.

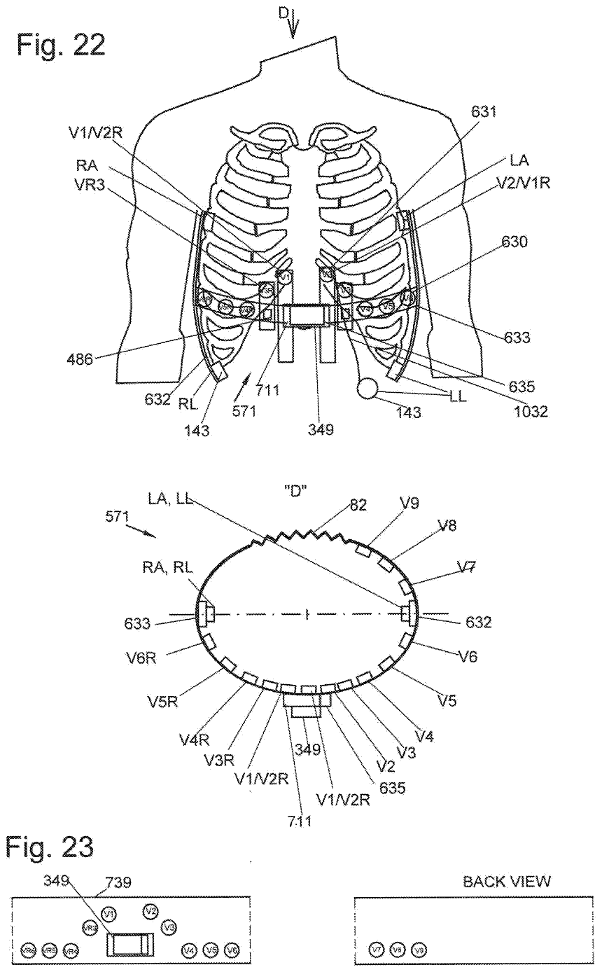

When using a multi-electrode base 630, shown in FIG. 22 signals of electrodes for sensing in points RA, LA, RL, LL, V6R, V5R, V4R, V3R, V1/V2R, V2/V1R, V3, V4, V5, V6, V7, V8, V9, therefore, therefore the leads I, II, V6R, V5R, V4R, V3R, V2R, V1R, V1, V2, V3, V4, V5, V6, V7, V8, V9 can be sensed at once, wherein, other 4 leads III, aVR, aVF and aVL can be calculated, what makes 21 leads totally.

For simultaneous sensing of 21 leads, a connecting field 711 with n=18 inputs is necessary for 15 chest leads, and for three signals LA, RA, LL.

The interconnecting field 711 is preferably modular and makes possible to connect a monitor or monitors according to the required number of simultaneously sensed channels, and this from 1 to 17. One multi-channel monitor or multiple monitors, for example three eight-channel monitor s can be mounted side by side. At the same time, it allows sequential sensing with switching over, as elucidated in examples of use in case of the base 720. The switch 700 KS/WS, which switches the electrode 653 for KS/LL to the electrode 197 LL, is used to select sensing against KS or WS. The KS has the advantage that all electrodes are placed on the sensing unit 571, preferably they are formed by a simplified base 720, preferably in the form of a belt, in case of the pseudo-KS, when the electrode 143 for KS is not applied, to pass on from measuring with KS to measuring with WS, it is sufficient to connect the electrode 143 for LL.

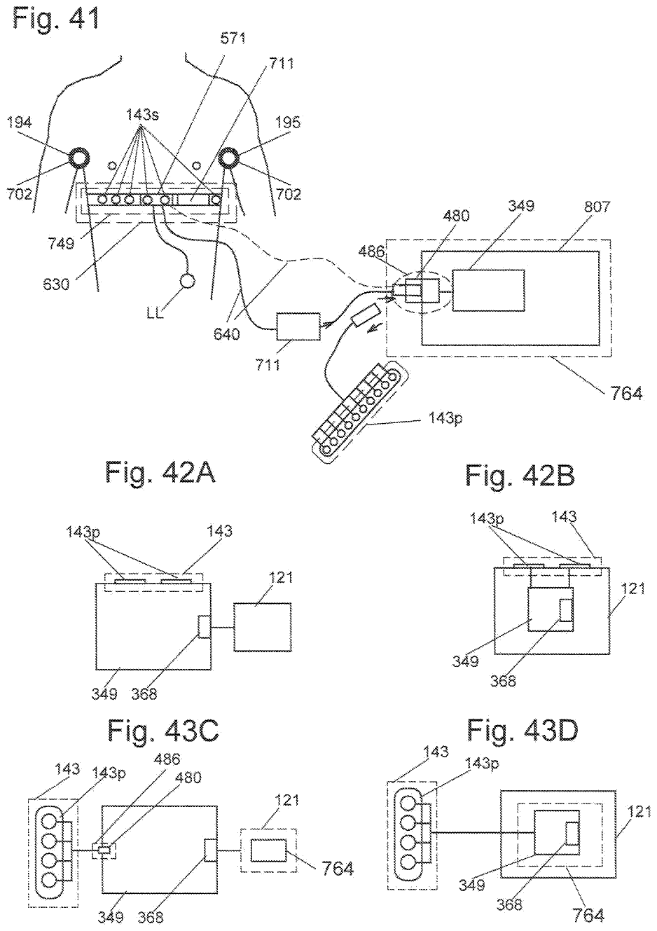

FIG. 22 shows using of the sensing unit 571, preferably formed by multi-electrode base 630 for sensing of up to 18-lead ECG with the help of the Wilson's central terminal. The multi-electrode base is based on the concept of a complete base 712 as shown in FIG. 41, but it is substantially more advanced. Directly on the base are located the electrodes 143 so that they abut to places recommended for sensing of V4, V5, V6, V7, V8, V9, V4R, V5R, V6R, and alternatively also the electrodes 143 for sensing of the spare signals RA, LA. All these points are situated approximately on a connecting line led around chest of a standing person in the same height. The signals of leads that are off this connecting line, i.e. the V1/V2R, V2/V1R, V3, V3R, are sensed by means of electrodes 143 situated on the transverse straps 631, and further, the signals/leads RA, RL, LA and LL are sensed with the help of the side belts, particularly, signals LA and LL by the side belt 632, and the signals RA and RL by the other right side belt 632.

Alternatively, a wide chest belt 739, shown in FIG. 23, is used.

FIG. 23 shows use of a wide chest belt 739, wherein, the upper line 636 of this wide belt is shown in this figure. Then, the electrodes V1/ V2R, V2/V1R, V3, and V3R are a part of the belt, and the transverse straps 631 with electrodes are not necessary.

All electrodes 143 are routed to a multiple lead switch 635, which is connected to a connector field 634. A multi-channel monitor, an eight-channel monitor 470, or even a multi-channel monitor can be connected to the connector field 634 via a connector, Depending on the connected monitor or ECG device and the demand for sensed leads, it is switched with the using the multi-switch 635 of leads, which, preferably, is controllable remotely.

If completely equipped this arrangement allows instant switching between a standard 12-lead sensing of ECG to sense reverse leads, or to sense rear leads.

Preferably, the side belts 632 are removable, and/or at the junction pivotally connected with the multi-electrode base 630 to minimize dimensions during transport or storage.

In case of removal of the belts 632, instead of electrodes 143 for LA and RA, electrodes 194 are preferably used for the spare signals LA′, and RA′. For LL, an electrode 143 located in the left abdominal area, connected by a cable 1032, is preferably used.

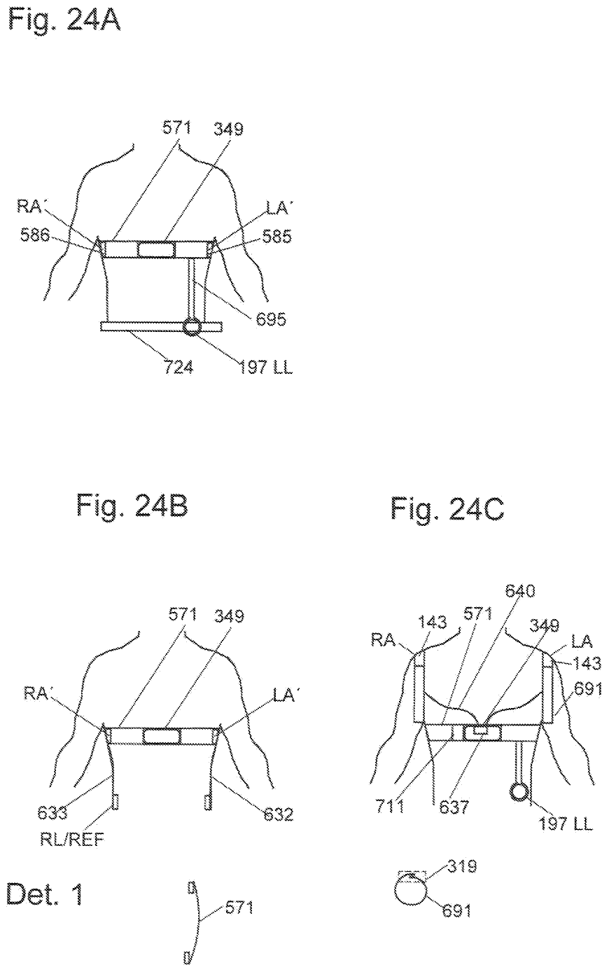

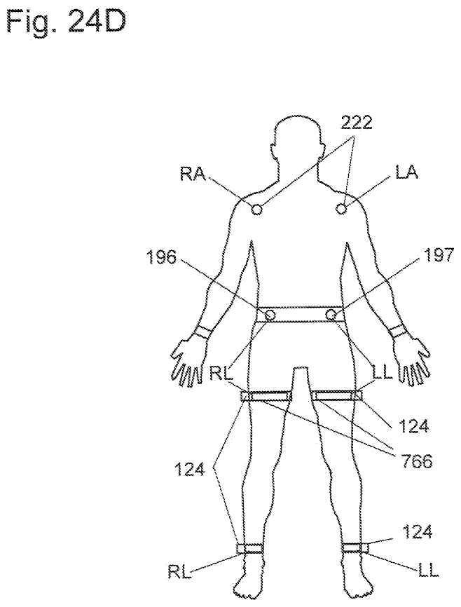

FIG. 24A and further FIG. 24B to FIG. 24D show well-arranged methods of obtaining potentials for the Wilson's central terminal 428 by means of electrodes 143, preferably formed by electrodes 686 for potentials RA, LA and LL, consisting of an electrode 197 for LLa, an electrode 687 for potentials from the left arm and the right arm preferably formed by the electrodes 195 LA and 194 RA, or by the electrodes 585. In this case, the electrode 196 RL is located on the abdominal belt 724, but it can be located also in other places, for example on the sensing unit 571 in the form of a belt or a small-plate.

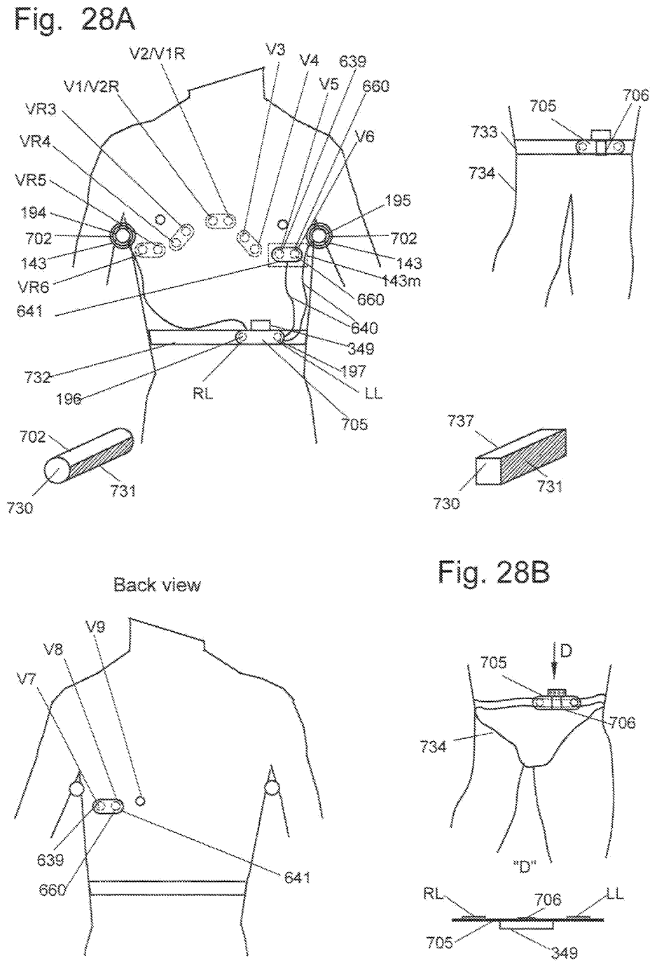

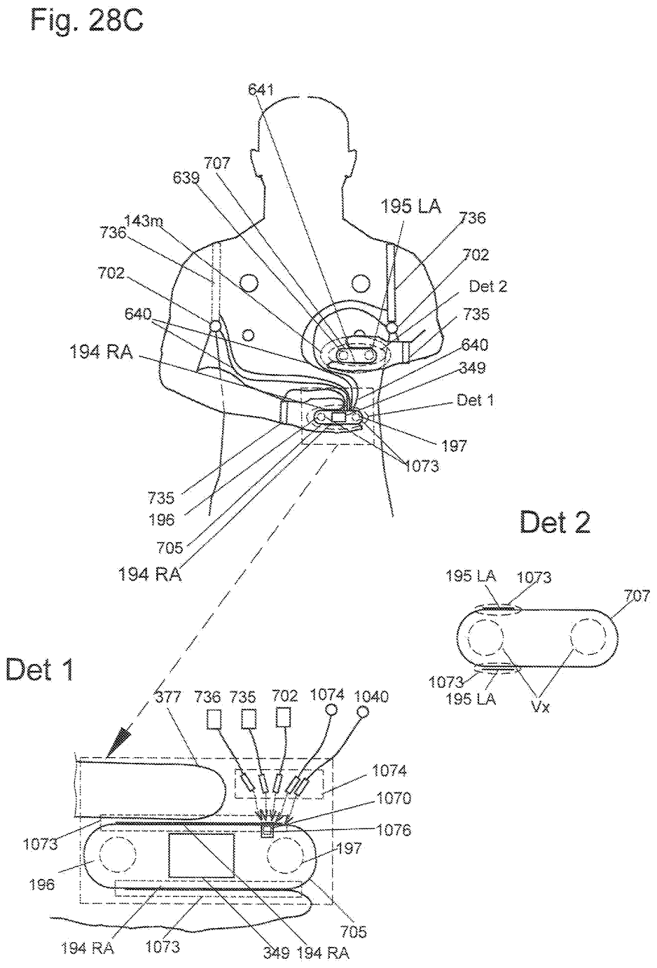

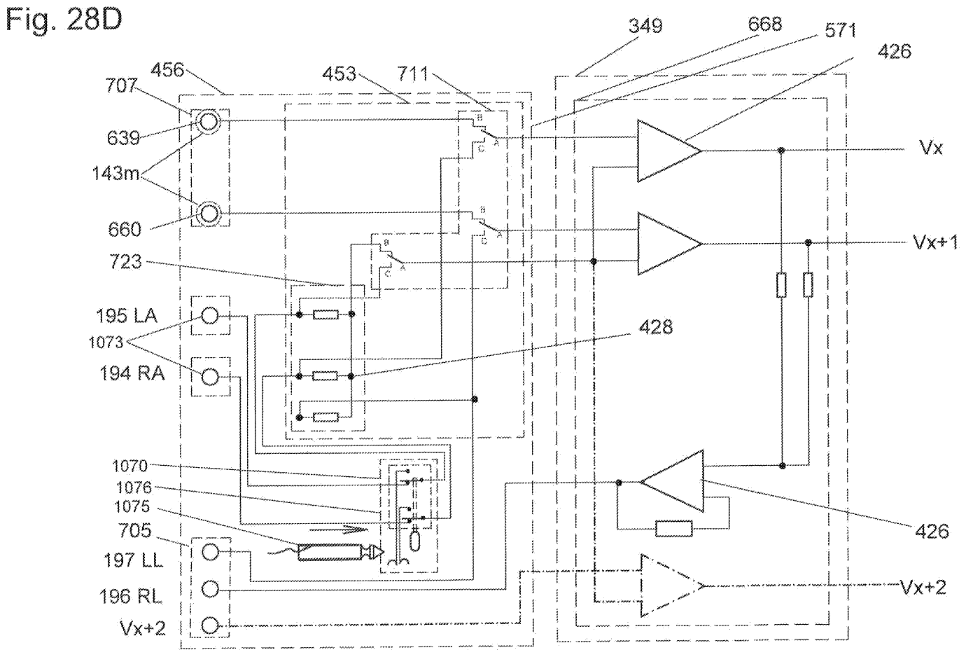

FIG. 24A shows the electrodes 586 RA′ and 585 LA′, which are located, for the sake of fixing simplicity, on sides of the sensing unit 571 in the form of a belt, preferably formed by a base 720 or a multi-electrode base 630, which is located on the lower edge of the sternum. The signals obtained here do not correspond exactly to the signals sensed from the electrodes 194 RA and 195 LA in the recommended places on the left and the right arms, but this small inaccuracy is balanced by the simplicity of attaching the electrodes 586, 585, which are located directly on the sensing unit 571 in the form of a belt. The electrode 143, formed by the electrode 197 LL is located on the strip 695 of the abdominal electrode, which is connected electrically by strip 695 of the abdominal electrode, preferably removable, to the base 571, and this arrangement makes possible to place the electrode 143, preferably formed by the electrode 197 to the place, which is recommended for sensing of LL in the left side of the abdominal area. The sensing unit 571 in the form of a belt is fastened to the chest by the elastic part 82 of the belt. Preferably, the electrode 197 LL is fixed to the abdomen with the abdominal belt 724, or it is inserted for example behind the trousers 734, as it is shown in FIG. 28B.

FIG. 24B shows use of the side belts 632 for location of electrodes 143 for obtaining of the potentials RA, LA, LL connected mechanically as well as electrically, preferably removable sensing unit 571 preferably in the form of a belt by a connecting element 696, by which they are connected to the monitor 349. Preferably, the side belts 632, 633 are from some elastic material, slightly bent, as it is shown in Detail 1, so that in its pressed state by the base 571 in the form of a belt they abut by their ends, where the electrodes 686 are located, to the body.

FIG. 24C shows use of shoulder straps 691 with electrodes 687 for RA and LA. The shoulder straps 691 are connected by a cable 640 and a connector 740 to the monitor 349, preferably via a connecting field 711. After wrapping around the shoulder, the straps 691 are preferably secured with Velcro or made of an elastic material for putting on an arm. Preferably, they are from a conductive material, preferably formed by metal small-plates, which are attracted to each other by a spring mechanism and form the electrodes 689 with their entire surface.

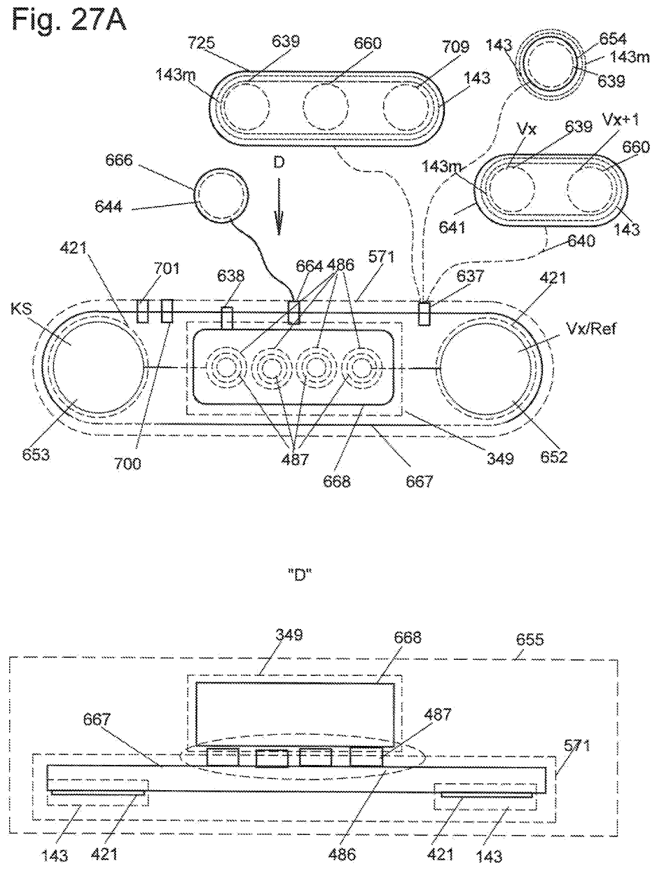

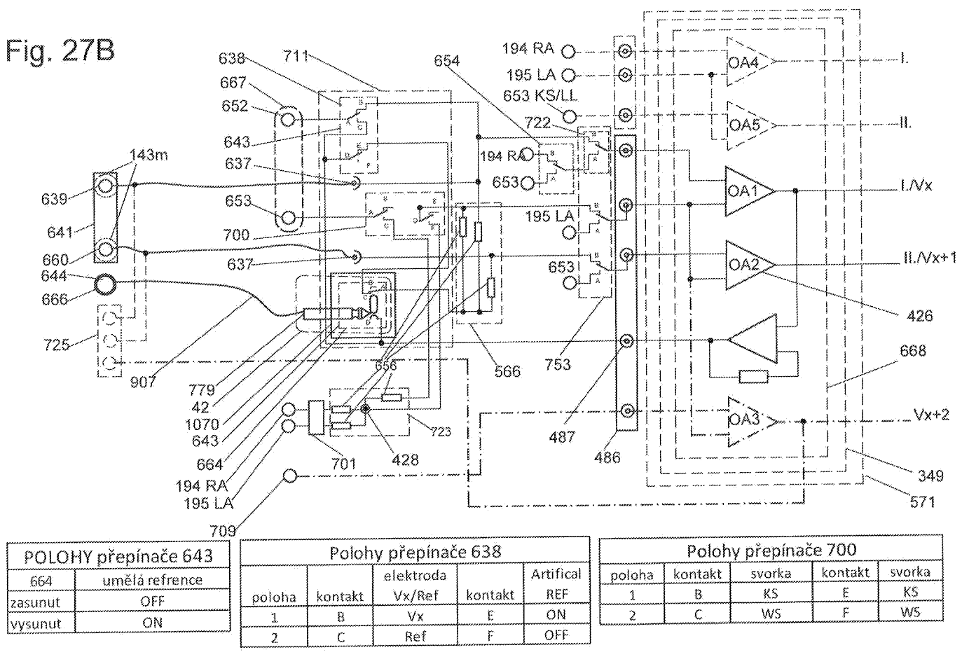

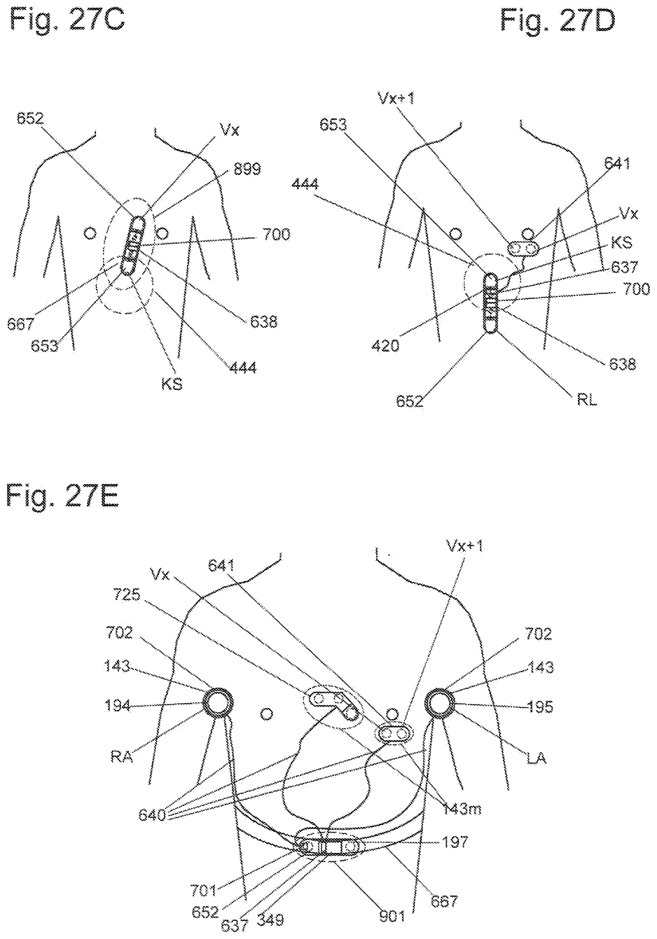

FIG. 24D shows obtaining of the RA, LA and LL potentials in conventional ways, i.e. by clips 124 on the limbs and/or by glued electrodes 222. An alternative obtaining of the RA, LA potentials is by hand contact with the electrodes 194, 449 shown as an alternative in FIG. 6, or with the volumetric electrodes 702 shown, for example, in FIG. 27E.

In this example, the electrode 197 LL is placed on straps 766 on the thighs or an ankle. The reference signal RL is either artificially produced in the monitor so as it is shown for example in FIG. 21F, or it is applied only by the electrode RL 196 on the sensing unit 571, preferably in the form of a belt or a strip 776, or on clamps 124, or they are glued. The electrode 196 RL can be placed at suitable locations shown in the figures of this patent.

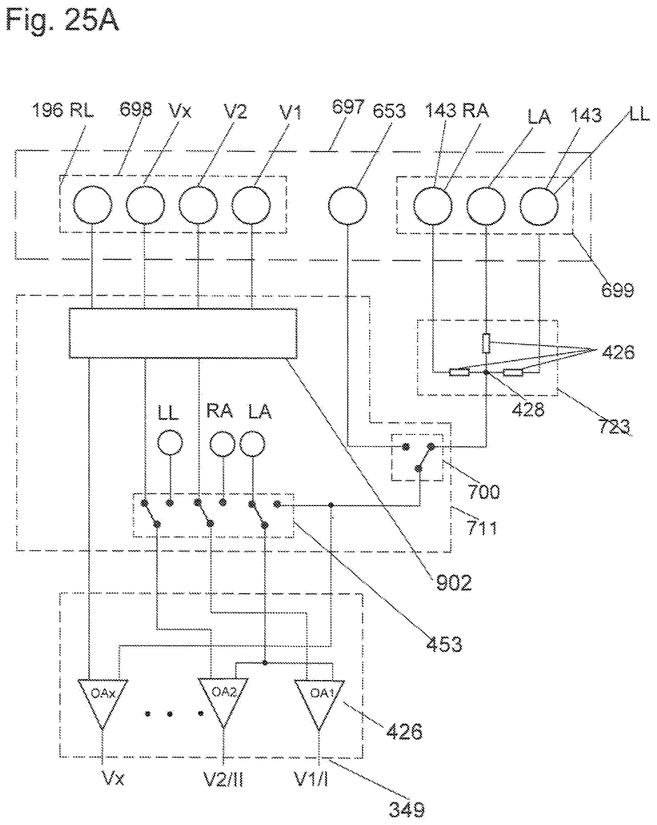

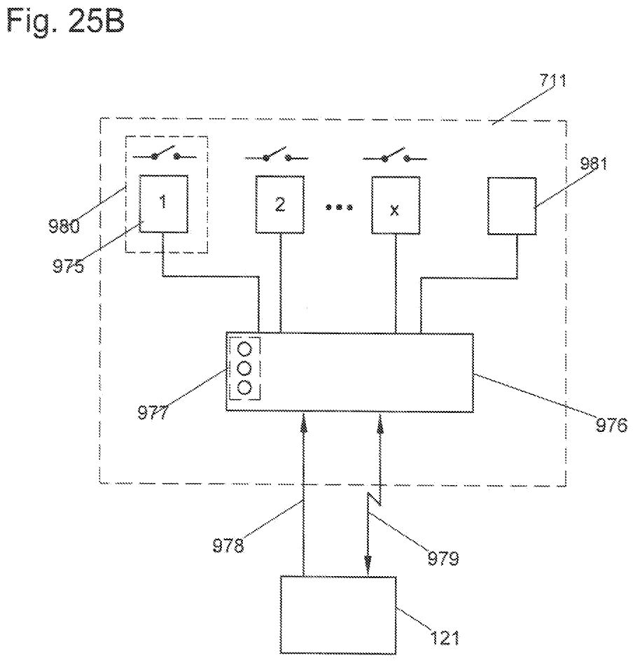

FIG. 25A shows an interconnection field 711 connecting to the input circuits of the monitor 349, preferably consisting of operational amplifiers 426, electrodes 143, preferably formed by an electrode set 697 consisting of a chest lead electrode set 698 from a set of WS electrodes 699 and when no artificial reference is used inside the monitor 349 to the electrode 196 RL.

In this example, the interconnected field 711 consists of an interconnecting field 902 for Vx, through which the selected electrodes can be connected to the inputs of the operational amplifiers 426 of the monitor 349.

Furthermore, the interconnecting field 711 consists of a switch 700 KS/WS, which can be used to switch to this input of operational amplifiers the clip 428 WS or the electrode 653 for the Kranz's central terminal and thereby to switch the sensing with KS to sensing with WS. Further, the interconnecting field 711 consists of a switch 453, which can switch over the inputs of two operational amplifiers 426 from sensing of leads Vx to sensing of leads I and II, in case of the two-channel monitor 349, In case of the mono-channel monitor 349 switch 454 LL/RA is used for switching over of electrodes 143 for sensing of both leads I and II, as it is shown in FIG. 9. The interconnecting field is formed by manual switches, as it is shown for example in FIGS. 25A, 9, or 27B.

Alternatively, instead of the switch 700, the monitor switches from the KS sensing mode to the WS sensing mode by relocation from the sensing unit 571 for KS, which is formed preferably by base 667 from FIG. 16 to sensing unit 571 for WS, preferably formed by the baseou 705 from FIG. 28A.

The electrode set 699 for the Wilson's central terminal is formed by three electrodes 143 for RA, LA and LL.

The electrode set 698 for the chest leads is usable for sensing of up to 15 chest leads, V1 to V6, V1R to V6R, V7 to V9, wherein, V1R=V2 and V2R=V1. With the help of the electrode set 699 for the Wilson's central terminal, i.e. of the electrodes for RA, LA and LL, it is possible to sense leads I, II and calculate the leads III, aVR, aVF, and aVL, thus obtaining up to 21-lead ECG by sensing of signals from 15 electrodes 143 by two-channel monitor 349 shown in the block diagram shown in FIG. 21F, to which monitor the connecting field 711 switches the respective electrodes successively.

It is possible to use a multi-channel monitor, thus reducing the number of switching through the connecting field 711. A 17-channel monitor would sense all 21 leads at once. In such case the interconnecting field 711 would not be necessary. For a 12-lead ECG, it is enough to have for the simultaneous sensing an 8-channel monitor 349 shown in FIG. 15. In case of the mono-channel monitor 349, it is necessary also to switch the electrodes 143 for signals RA and RL by the switch 454, as it is shown in FIG. 9.

FIG. 25B shows alternative embodiment of switches of the interconnecting field 711 by means of switching elements 980 formed by relays 975 controlled by the control unit 976 of the interconnecting field, preferably formed by the control unit of the monitor 349, which is controlled preferably by push-buttons 977 of the interconnecting field.

An advantage is that for certain sensing, for which it is necessary to switch over more switches it is sufficient to press one push-button for the respective sensing, which switches more appropriate relays and simplifies the switching over.

Another advantage is that the relays 975 are controlled by the control unit 976, which can be connected by wire interconnection 978 or wireless interconnection 979 to the cooperating unit 121 shown in FIG. 9, by which thy can be controlled remotely, and thus chosen sensing of the required leads, preferably sensing with KS or WS. Preferably, the switching elements 980 can be formed by electronic switches 981.

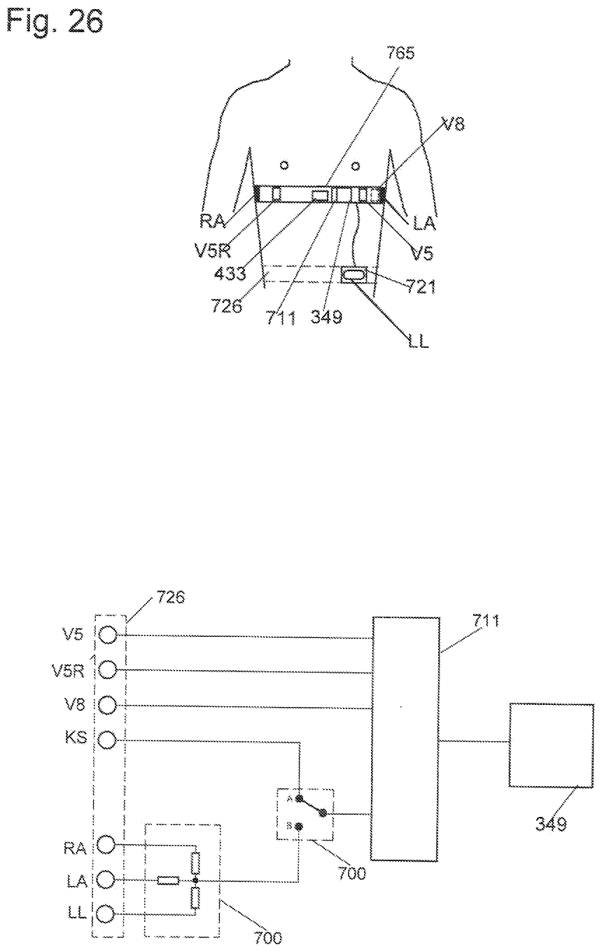

FIG. 26 shows switching over between sensing with the Kranz's central terminal and sensing with the Wilson's central terminal 428 on the example of a sensing unit 571, preferably formed by base 765 in the form of a belt. The electrodes 143 for V5, V5R, V8, KS of the base 765 are interconnected preferably to the interconnecting field 711, wherein, the electrode 143, preferably formed by electrode 418 of the base for the area of the Kranz's central terminal, is connected to the switch 700 KS/WS.

From the base 765, the signals of electrodes 143 for RA, LA are also fed to circuits 723 of the Wilson's central terminal 428 together with the signal of the electrode 143 for LL from the small-plate 765 and the WS 428 is brought to the interconnecting field 711 to the switch 700 KS/WS.

On the switch 700 KS/WS, it is switched between KS and WS. The interconnecting field 711 switches the respective electrodes optionally to the monitor 349. KS or WS are coming from the circuit and it is brought to the interconnecting field 711, which allows connecting the selected monitors 349.

The abdominal small-plate 720 for sensing LL is held by hand, or preferably, it is inserted under the trouser waist belt or strap 726 for LL.

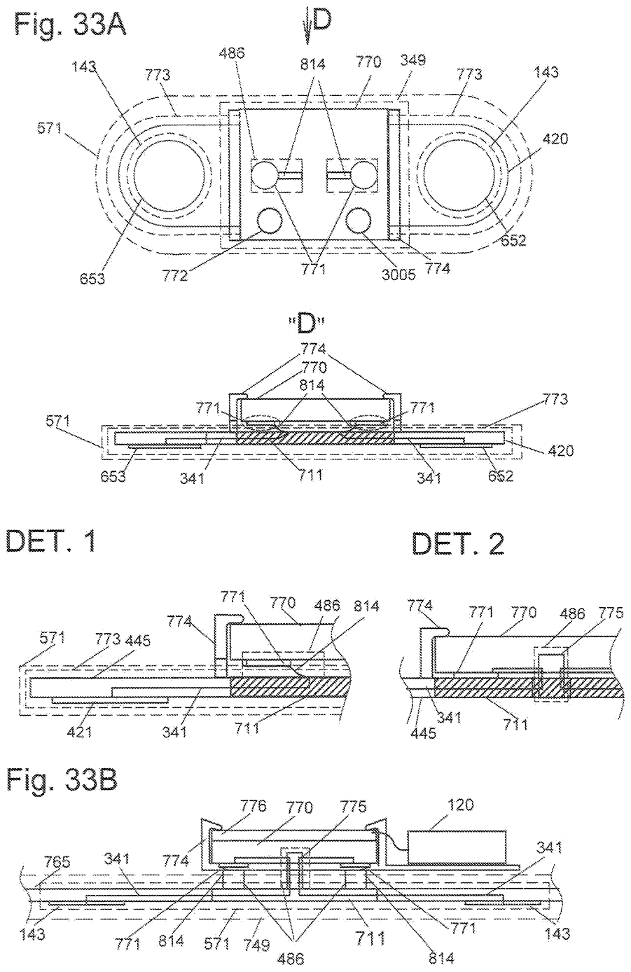

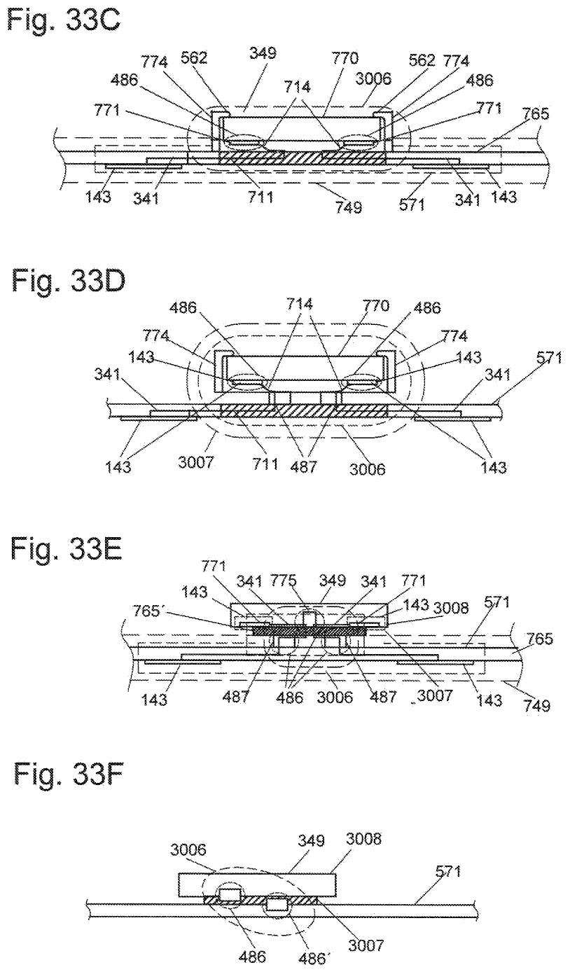

FIG. 27A shows mechanical design and FIG. 27B shows block diagram of a modification of the sensing unit 571, preferably formed by the base 667 in the form of a small-plate from FIG. 16 for the Kranz's central terminal for the possibility of switching over to the Wilson's central terminal with the switch 700 KS/WS, by which it is connected to the monitor 349 from the z electrodes 653 for KS/LL to the circuits 582 of the Wilson's reference connected through the connector 701 for WS, and the electrode 653 KS/LL is connected to sensing of LL for WS. The variants of location and connection of the electrodes for RA, LA, and LL, which are used for circuits WS, are shown for example in FIG. 33E, wherein, the electrodes 194 RA and 195 LA are connected via the connector 701 for WS.

The electrode 652 is connected to reference RL by switch 638 Vx/REF and the chest leads are sensed by electrodes 639, 660 on the small-plate 641, connected via the connector 637 for Vx.

Alternatively, instead of the monitor 668, the monitor 770 with electrodes from FIG. XXX is used.

The switch 700 KS/WS, switches the connection of the electrode 653 for KS/LL to connection for LL, and further it connects the Wilson's central terminal 428 to inputs of both operational amplifiers of the monitor 349. The base 667 is moved from the position on the chest area, shown in FIGS. 27C, 27D to the abdominal area to the position WS 901 for sensing LL by electrode 653 for KS/LL “and for applying of the reference RL through the electrode 652 Vx/REF “, as it is shown schematically in FIG. 27E. Optionally, it is possible to use the switch 722 (I, II)/Vx, which allows sensing of Vx in the position B and by switching over to the position A and to sense also the leads I and II and to calculate leads III, aVR, aVL aVF from them.

In case, the switch 722 is not used, in the block diagram, the contact B is connected permanently to the contact C, and it is possible to sense only the leads Vx against KS or WS. Optionally, instead of the switch 722, another two operational amplifiers 426, marked OA4 and OA5 can be used, which allow to sense leads I and II continuously and to calculate 4 leads from them. When sensing the leads I and II, the switch 700 KS/WS is switched to the position C for WS and the base 667 is moved to the abdominal area, to that the electrode 653 senses the LL.

In the basic version two operational amplifiers are used, and this for the OA1 and OA2 for sensing of the chest electrodes Vx and Vx+1 with small-plates 641. The switch 722 is not used. By adding of the third operational amplifier 426, the OA3, is made possible to sense three chest electrodes Vx, Vx+1, and Vx+2 simultaneously width help of the small-plate 725 of three chest electrodes.

By adding of another two operational amplifiers 426, OA4, OA5, it is possible to sense with three chest leads simultaneously also the leads I and II. By using only one operational amplifier 426 OA1, for example when using the monitor 770 from FIG. 3, it is possible to sense only one chest lead Vx, preferably with the small-plate 654, simultaneously the electrodes on the cable, and by and relocation of the small-plate, it is possible to sense all left side, right side and back chest leads successively, and by using the switch 722 also the lead I and successively also the lead II by adding of the switch 654 LL/RA and from them it is possible to choose by software sequences of pulses with identical pulse rate, to determine from them by calculation the them corresponding sequences for another four leads III, aVR, aVL, aVF, and to complete so the ECG recording of the individually sensed leads for complete sequence corresponding to a twelve-lead ECG. For use of the reference electrode 644 the cable 907 of the connector is inserted into the connector 664 of the reference, and it disconnects the artificial reference and connects the electrode 644.

FIG. 27C shows the method of sensing the chest leads with the help of the base 667 of individual chest leads with electrodes 652 Vx/REF against the Kranz's central terminal. The base 667 is in the position 899 KS. The switch 700 KS/WS is in the position 1 for KS and the switch 638 is in the position 1 Vx.

The electrode 653 for KS/LL is placed into the area 44 of the Kranz's central terminal and the electrode 652 Vx/REF is moved to positions recommended for sensing V1 to V6 and V1R to V6R.

FIG. 27D shows the method of sensing the chest leads with the KS base 667 of the chest leads in pairs with the help of the small-plate 641 and with the possibility to use reference. The base 667 is in the position called position 900 KS with and small-plate, the switch 700 KS/WS is in the position 1 for KS, the switch 638 is in the position 2 REF. The base 667 is placed with the electrode 653 on the lower verge of the area 444 for the Kranz's central terminal and with the electrode 652 Vx/REF in the direction downwards to the abdomen area. This electrode is used for reference. To the connector 637 of the base 667 is connected the small-plate 641 for a two-channel monitor with two electrodes for sensing Vx and Vx+1. This small-plate makes possible to sense any two adjacent leads Vx against the Kranz's central terminal at once with the help of a two-channel monitor 349, preferably formed by the monitor with the taken out reference 668.

The FIG. 27E shows location of the base 687 for sensing with WS to the position 901 WS and of electrodes 194, 195 in the armpits, the small-plate 641 on the chest and the position of switches 638 in the position 2, when the electrode 652 serves as reference RL, the switch 700 KS/WS in the position 2, when the electrode 653 serves for LL, for switching of the base from sensing with the Kranz's central terminal KS to sensing with the Wilson's central terminal WS. The base 687 is shifted to the abdominal area to the position 901 WS for securing of the reference RL with electrodes 652 Vx/REF switched over to REF and the sensing LL with the electrode 653 for KS/LL switched over for LL. The switch 700 KS/WS is in the position 2 WS, switch 638 is in the position 2 REF. Preferably, the base is held by hand, or it is fastened to the strip 732 for base. The signals RA and LA are brought to the base 687 via the connector 701 for WS, the cable 640 of electrodes 143, preferably formed by electrodes 194 RA and 195 LA in the armpits, and preferably, they are formed by the bulky electrodes 702. The chest and the back leads are sensed successively by attaching of a small-plate 641, as it shows FIG. 28A. The switch 638 Vx/REF is in the position 2 Ref, the switch 700 is in the position 2 WS.

FIG. 28A shows an example of a change in sensing of ECG by means of the KS terminal to sensing by means of the WS terminal by relocation of the monitor 349 from the base 667 for the KS, as shown in the FIG. 16 located on the chest to the base 705 for LL, RL placed in the waistline. Preferably, the electrodes 143 formed by electrodes 194 for RA and 195 for LA are located in this example in the armpits, and preferably they are formed by bulky electrodes 702, preferably formed by cylindrical electrodes 703 or spherical electrodes 704, or cuboidal electrodes 737, and preferably they are made of a soft elastic material, for example of rubber foam, so that by their elasticity they allow moving of the arms away from the body without loss of contacts of electrodes width the body or that they would fall out. Preferably, the bulky electrodes 702 are oriented so that their active conductive part is oriented to the body and senses so the signal only from the chest, where there is less disturbing signals than as in the limbs. The bulky electrode 702 consists of elastic filling 730 of the electrode and of the conductive part 731 of the electrode preferably covering a part of the filling 730. The electrodes 702 are connected by cables 640 with seethe “base 705 for LL, RL” held by fingers or they are fastened to the waist belt 726 for RL, LL in the area of waist or this base 705 is inserted under the trousers waist belt 733, and preferably it is secured by the clip 706 to the trousers waist belt or it is inserted behind the knickers 734 of the underwear. The signals from electrodes 143, preferably formed by electrodes 639 for Vx and 660 for Vx+1 for the chest leads Vx and Vx+1, located on the small-board 641 of electrodes for the two-channel monitor, are brought by a cable 640 into the base 705, where the monitor 349 is situated.

The cable 640 is sufficiently long or extendable, so that the small-plate 641 can reach not only the chest leads, but also the back ones V7 to V9, where they are, after their placing held by hand, and preferably they are fixed on the required place so that for example the monitored person leans on the chair. Alternatively, the monitor is placed on the base 641.

In these configurations, leads V1 to V6, V1R to V6R, V7 to V9 are sensed by placing the small-plates 641 at the respective locations for sensing of pairs of these leads.

FIG. 28B shows shape adaption of the base 705 for LL, RL for positioning of the monitor and inserting it behind knickers 734, which provides pressure to the electrodes 143 for RL and LL. The fitted monitor 349 or the buckle 706 prevent the base 705 from falling under the trousers waist belt. Preferably is base is fixed in the required position by a waist belt clip 706.

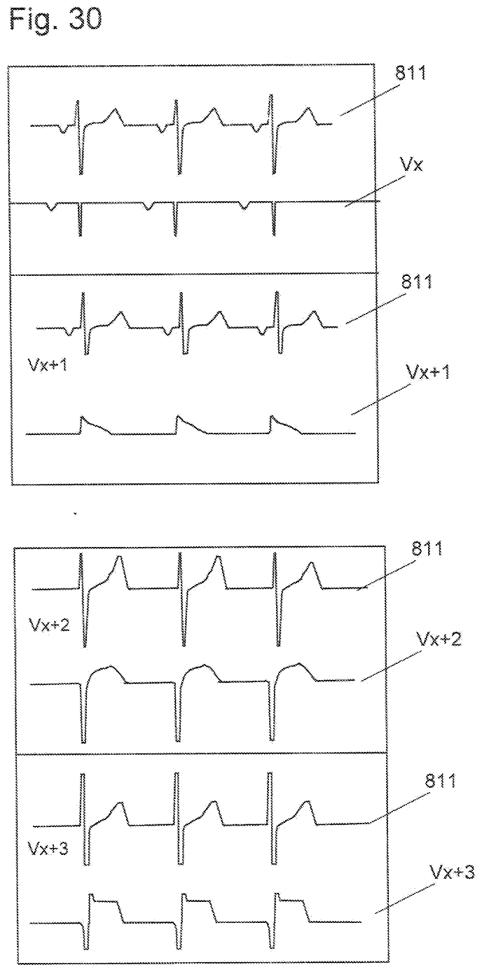

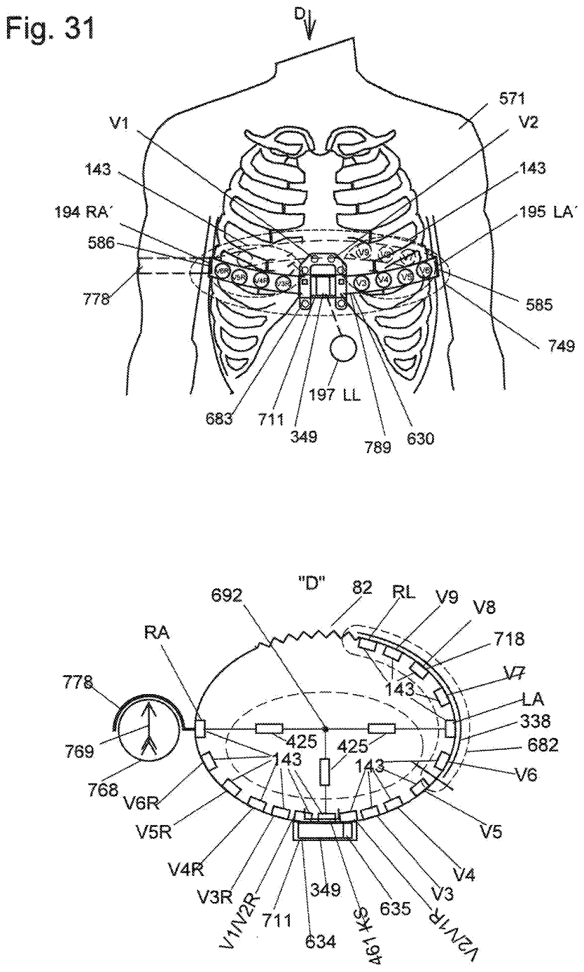

FIG. 28C shows arm positions for sensing of the chest leads against the Wilson's central terminal using at least one type of electrodes for LA, RA from; wristband 736 for a shoulder, wristband 735 for a wrist, bulky electrodes 702, glued electrodes 1074, contact electrodes 1073 for finger shown in Detail 1,2. The monitored person places the respective electrodes, for example the bulky electrodes 702 to armpits or with fingers touches the contact electrodes 1073, 194 RA and 195 LA, the bulky electrodes 702, the glued electrodes 1074, the wristband for a shoulder 736, the wristband for a wrist 735, which are connected to the base 705 preferably with the connector 1070 of the jack type, which, when the pin connector 1075 is inserted, by the connector switch 1076 disconnects the contact electrodes 1073, which are formed preferably by electrodes 194 RA and 195 LA on the base 705 and connect the respective electrode, connected by a cable 1014. When the monitored person does not use the possibility of inserting the base 705 behind the trousers waist belt, so the monitored person holds it, preferably with the right hand, on abdomen. Preferably, with the left hand, the monitored person moves the small-plate 641 with two electrodes to the respective points for sensing of the chest leads Vx. An advantage of the bulky electrodes 702 consists in that they are resilient, wherefore thy change their shape. They adapt to the gripping strength between the arm and the chest, and therefore, they are not a hurdle for the monitored person, because they allow wide extent of the arm movement, and at the same time, they provide the necessary permanent contact with the chest skin. Alternatively, instead of the volume electrodes 702, it is possible to use for the LA, RA the wristbands 735 for a wrist or the wristbands 736 for a shoulder, which are, preferably, connected by a cable to the connector 1070 of the jack type, which when inserted into the female connector, disconnects the electrodes 195 LA on the small-plate 641, which is held by fingers of the right hand or the electrodes 194 RA on the base 705, held by fingers of the left hand. When the connector 1070 is not connected, the electrodes 194 RA and 195 LA are used for RA, LA. The wristbands 735, 736 are resilient, preferably they are made of some resilient material, preferably from rubber, or from metal plates 1049, which plates are mutually attracted by springs, so that the wristband 735 springs and abuts the skin, or they are provided, for example, with a Velcro for fastening with a belt, and they are made of some conductive material, to form contact with the body. In this example the monitor 349 is made as a two-cannel and/or a single-channel one, preferably it is formed by a monitor 770 with electrodes, as it is shown in FIG. 31, and therefore, it is used only one operational amplifier 426, marked as OA1 and in sensing of individual chest leads Vx the small-plate 654 is used.

FIG. 28D shows a block diagram of a change from the sensing with the Kranz's central terminal to sensing with the Wilson's central terminal by repositioning of the monitor 349 from base 420 to the “base 705 for LL, RL”. The signals from electrodes 639 for Vx and 660 for Vx+1 are brought into the monitor 349, which is connected to the base 705 for LL, RL by connecting elements 486 preferably formed by snap fasteners 487. The chest leads Vx are sensed against the Wilson's central terminal. Therefore, also the signal from the circuits 723 of the Wilson's central terminal, which is formed with the help of the signals from electrodes 194 RA, 195 LA, and 197 LL, is brought to the monitor 349.

At the same time, the feedback reference signal from the input circuits of the monitor, which is applied to this electrode 196 RL, is used.

The switch 722 I, II/Vx of leads switches the signal to the monitor 349 from the position B therefore of the electrodes 639 for Vx and 660 for Vx+1 to the position C, therefore, to the electrodes 194 for RA and 197 for LL and from the Wilson's central terminal 428 to the electrode 195 for LA for sensing of ECG leads I and II, from which leads III, aVF, aVR, and aVL can be calculated, whereby, in this position of the switch 722, totally 6 ECG leads are obtained.

In the position of the switch 722 for Vx, therefore, in the position B, it is sensed by successive attaching of the base 707 for WS with two electrodes 639, 660, with the help of which it is possible to sense, a pair at a time, successively the leads V1 to V6, V3R to V6R, V7 to V9, that is 13 chest leads. From the leads V1 and V2, it is possible to derive the leads V1R and V2R, whereby, the number of chest leads will be increased to 15. Another two leads are sensed in the position of the switch 722 for the leads I and II, from which leads, the leads III, aVF, aVL and aVR are calculated, that is four another leads, i.e. together 6 leads. Therefore, the total number is 21 leads sensed successively by a two-channel monitor 349, specified in this example in approximately ten locations. If a multi-channel monitor 349 and the base 707 for WS for more electrodes 143 would be chosen, the number of relocations would be respectively lower.

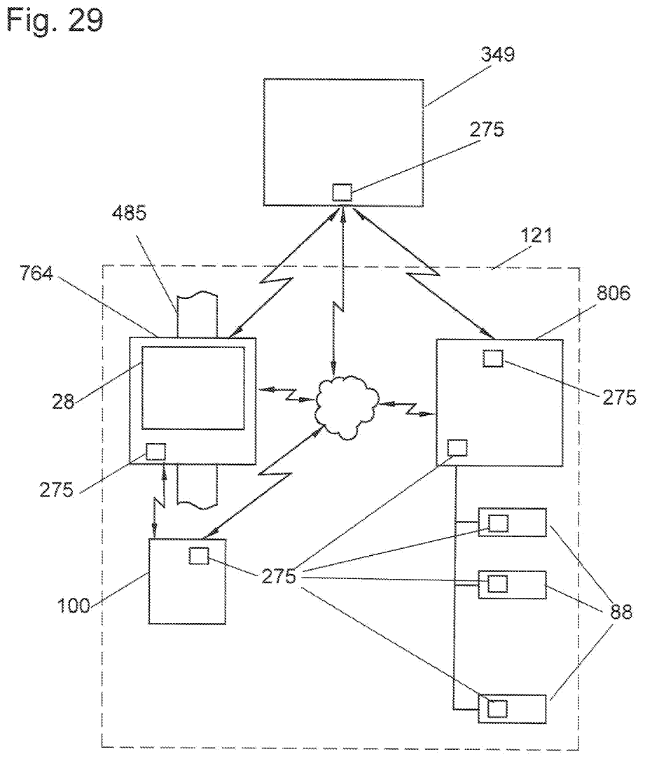

FIG. 29 shows communication of units, providing operation and full utilization of the handheld monitor 349. Preferably, the monitor 349 communicates wirelessly by means of a communication unit 275 with the cooperating units 121, preferably formed by the evaluating unit 764. Further, preferably, the monitor communicates with a server 806, particularly via a network 898 of a mobile operator or via a WiFi network 131 or via a direct wireless link. Preferably, the evaluating unit 764 is formed by a mobile phone 100, or it is on the wristband 485 connected with the mobile phone 100, preferably wirelessly. The evaluating unit 764 shows results on the display 28, preferably, it controls also the monitor 349. Further, the unit 764 is preferably adapted for direct wireless connection with a server 806. The processed, sensed cardiac signals are available also in the server 806 for remote participants 88 and the service staff of the server 806, which have access to the results, and at the same time can set up the monitor, and this either directly, or through the evaluating units 764.

FIG. 30 shows displaying on a display of the evaluating unit 764, preferably formed by a mobile phone, where, when using for example a two-channel or a multi-channel monitor se on every transmission of the cardiac signal data from the place for certain chest leads Vx, initialized preferably with a push-button 750, their displaying on the monitor 349 or on the sensing unit 571, or on the evaluating unit 764, preferably after every placing of the base in a new place on the chest or on the back, preferably two or more ECG curves are displayed for the leads actually sensed, and preferably, a sample ECG curve is displayed above them or inserted in them, which curve displays the normal ECG for the respective lead. Preferably, the sample ECG curve 811 for the required lead is produced in the time period when the monitored person exhibits standard course or a course without myocardial infarction (MI).

Accordingly, the monitored person evaluates whether the sensed course differs, especially with respect to the elevation of the ST segment. If the current course coincides with the exemplary one, there is no suspicion of the possibility of an infarct.

In case of difference, the monitored person evaluates the ECG according to the instructions, especially with regard to possible infarct and/or sends the recording from the monitor 349 via communication unit 275 or evaluation unit 764 directly or via server 806, and from there to remote participants 88, who evaluate the recording or a live transmission, and notify the monitored person. Preferably, the ECG image can be sent from the mobile phone unit 764, preferably as an image of the display via MMS.

Preferably, each captured ECG record for individual leads is stored in memory, and the unit 764 is adapted for sequential displaying of the selected leads, preferably by scrolling with a push-button or, in the case of a touch keyboard, with a finger.

The data transfer from the monitor, preferably to the unit 764, is initiated preferably by a push-button 753 for transmission, preferably after each attaching or relocating of the sensing unit 571 to the desired location for sensing of cardiac signal.

FIG. 31 shows a sensing unit 571, preferably formed by a chest belt 749, preferably formed by a multi-electrode base 630 in the form of a belt, preferably for sensing with a strengthened KS, up to 11 chest leads, V3 to V6, V3R to V6R, V7 to V9, wherein, all electrodes 143 are located on the base 630, with the possibility to add electrodes located off the base 630, preferably the electrodes 197 LL for sensing with a pseudo-Wilson's central terminal 694, see FIGS. 20A, 24A, and electrodes 194 RA, 195 LA, allowing sensing with the Wilson's central terminal 428, shown for example in FIGS. 22, 26, 32A, preferably with a bridge 683. The interconnecting field 711 connects the respective electrodes 143 to at least one monitor 349 for sensing of the chosen leads. The multi-electrode base 630 is attached and held on the chest and the back, preferably with the help of an extension 778 for an arm, which presses the belt 749 to the chest in the direction of the arrow 762 in the direction of the hand pressure and of the extending part 682 of the base, preferably formed by a strut 338, held by leaning of the monitored person against a chair, as they are described in the Example shown in FIG. 25A. Alternatively, the multi-electrode base 630 is permanently fastened to the trunk by the elastic part of the belt 82, which part is clipped to the belt 749, and the extensions 778 are disconnected.

To obtain strengthened terminals of the Kranz's central terminal 692 electrodes 585 LA′ and 586 RA′, are used, which are situated on sides of the multi-electrode base 630, in the places of sides of the monitored person that are connected with the help of three resistors 425 with the KS, as it is shown in FIG. 19A.

Alternatively, preferably the pseudo-Kranz's central terminal, obtained by means of two resistors 425 form the electrodes 585 LA′ and 586 RA′, situated on the chest belt 749, see FIG. 19B, or electrode 197 for LL for the Wilson's central terminal is connected so that the electrode 481 for KS is not necessary to be placed on the multi-electrode base 630, and instead of this electrode it is possible to place the electrodes 211 V1 and 212 V2 alias V1R, V2R, shown in dashed lines, which are added to the eleven ones for the chest leads above, whereby a complete set of fifteen chest electrodes is obtained.

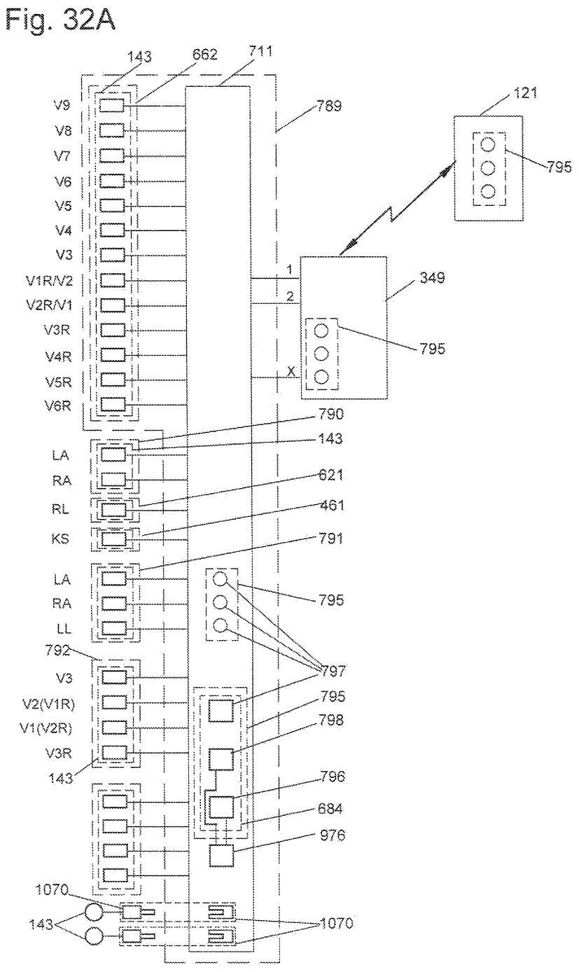

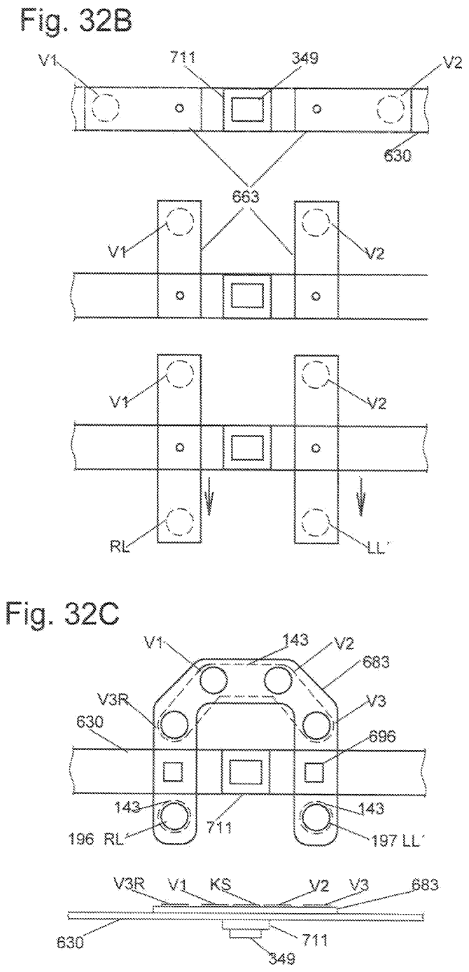

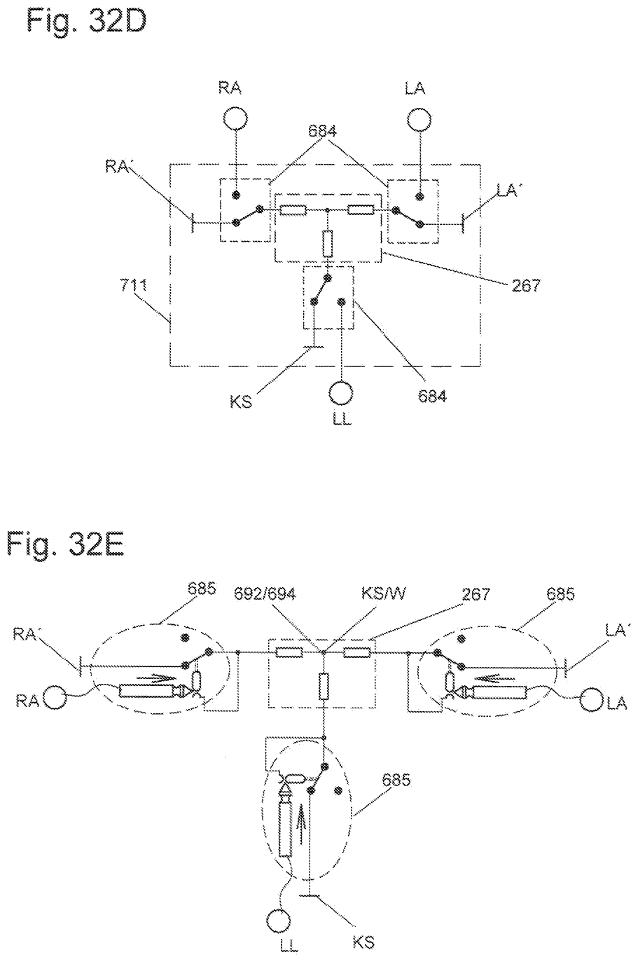

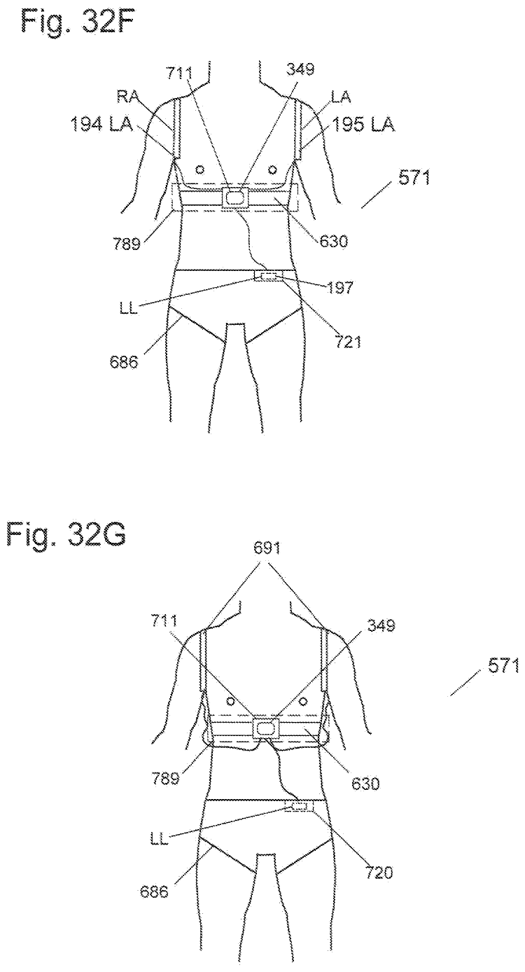

FIG. 32A shows electrodes 143, which are connected to the connecting field 711, which field is connected to at least one monitor 349 for sensing of the selected leads with a switch 684, which switches the field, preferably formed by the manual switches 797 and/or electric switches 796, preferably formed by push-buttons or electronic switches 798 controlled by the control unit 976 of the interconnecting field, preferably controlled from the controlling elements 795 of the interconnecting field, located on the connecting field 711 or on the monitor 349 or on the cooperating units 121. The electrodes 143 are located on or off the sensing unit 571, preferably formed by the base 789 in the form of a belt, preferably formed by a multi-electrode base 630 or by some of the bases 420, 620 for the RL, the complete one 712, the 765 in the form of a belt, the simplified one 720.

Preferably, the electrodes 143 placed on the base 789 are formed by electrodes 662 for the chest leads, by the electrodes 790 for the pseudo-Wilson's central terminal, by the electrodes 621 for the RL, by the electrodes 461 for sensing of potential of the Kranz's central terminal, and by electrodes 143, which are located off the base 789, which are preferably formed by the electrodes 791 for the Wilson's central terminal, by the electrodes 792 for the chest leads, by the alternative electrodes 793, see FIGS. 32B to 32F.

FIG. 32B shows the extension of the number of electrodes 143 at the base 789 by electrodes for leads V1, V2 to the leads sensed according to FIG. 32A, which are located on the rotatable strips 663. Both rotatable strips 663 are composed of two segments, the upper one and the lower one. In the working position described below, the connected upper and lower connected segments are tilted upwards, and in this position the lower segment is extended downwards. In this configuration, they snap and form a strap perpendicular to the longitudinal axis of the multi-electrode base,, which is pressed by the base to the chest. In the figure above they are drawn in the position, when they are not used, in which they are turned into the plane of the multi-electrode base 630, in the figure in the center they are turned upwards by 90° for introducing then into the working position, whereby, the electrodes for V1, V2 get into the sensing position, and in the bottom of this figure also the lower parts of the extractable straps 663 are pulled out, wherein they press the electrodes for V1 and V2 to the body so that they ensure attaching of the straps 663 to the chest so that the electrodes V1, V2 cannot tilt back. The electrodes for V1, V2 are connected to the monitor 349 through a connecting field 711, also so as preferably the electrode is connected for the LL instead of the electrode for KS, whereby we obtain signals, that are for orientation somewhere between the signals, that we would obtain by sensing against the Wilson's central terminal and the Kranz's central terminal. By connecting the electrodes 197 LL and by disconnecting the electrodes LL′, it is possible to sense against the Wilson's central terminal.

FIG. 32C shows connecting of electrodes 143 that are located outside the multi-electrode base 630, preferably formed by a chest belt 749, for V1 to V3 and V3R, and preferably by electrodes 143 for LL′, RL′ on the bridge 683, optionally fastened to the multi-electrode base 630 by a connecting element 696 to attain location of electrodes 143 on the chest for the specified places for sensing of ECG. The electrodes 143 are connected through a connecting field 711 as needed.

FIG. 32D shows connecting of external electrodes 143 for LA, RA from FIG. 32A instead of electrodes 143 for LA′, RA′ located on the multi-electrode base 630 and connection of electrode 143 for LL instead the electrode KS to obtain full valued sensing of chest electrodes against WS and sensing of the lead I and II of ECG with the possibility to calculate another four leads, whereby, up to 21 lead ECG is obtained, which is suitable for diagnoses of infarct also from the left side and the back leads. The electrodes 143 are interconnected by switches 684 of the interconnecting field 711. Optionally, the switch 684′ disconnects the electrode for KS as well as for LL for sensing with pseudo-Kranz's central terminal 693 as it is shown in FIG. 19A.

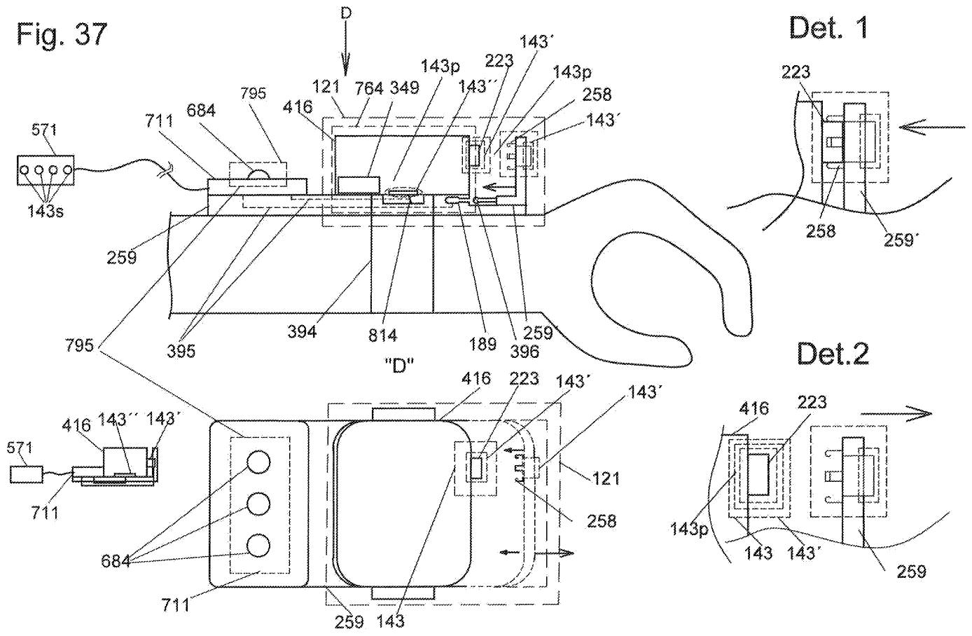

FIG. 32E shows alternative interconnection of electrodes 143 for LA, RA, LL with the help of the switching connector 685 from FIG. 37, which connector when inserted connects electrodes for example the LA and disconnects the LA′, and similarly for the RA, LL.

FIG. 32F shows connection of the electrode 197 LL located on the abdominal small-plate 721 for changing of sensing with the strengthened Kranz's central terminal 692 to sensing with the pseudo-Wilson's central terminal 694.

FIG. 32G shows the base 789 in the form of a belt preferably formed by the multi-electrode base 630 during connection of the external electrodes 143 for RA, LA, LL, where as an example were chosen for the RA and LA shoulder straps 691 with an electrode and for the LL the abdominal small-plate 720 inserted under underwear 886.

Therefore, in this example of embodiment the electrodes 143 are realized as a preferable alternative to glued electrodes, whose use is so possible to be avoided.

For sensing with the help of the full valued Wilson's central terminal 428, it is enough to connect the electrode 197 LL to the multi-electrode base 630, for example on the abdominal small-plate 721 and to connect the electrodes 194 RA and 195 LA, preferably placed on shoulder straps 691, as glued electrodes, or as electrodes on clips.

It is preferable that with the exception of electrode 197 LL and of electrodes 194 RA, 195 LA, all electrodes 143 to obtain sensing 21 of leads are on the multi-electrode base 630 in the form of a belt.

FIG. 33A shows a monitor 349 formed by the monitor 770 for attaching, used for sensing of signals, for storage space saving small size. Even though the electrodes 771 are located on the surface of the monitor 770 on its verge, the electrodes 771 are placed close to each other, so that they allow only sensing of cardiac signals from a small space of the chest, which signals do not correspond to any ones from the twelve leads of the ECG leads standard, and they are suitable only for some limited evaluation of the cardiac activity, which however is sufficient for the basic evaluation, and this limitation of the diagnostic possibilities makes possible to use a monitor 770 of small size, preferably of the storageable size of a credit card.

To expand the diagnostic possibilities and at the same time to maintain the advantageous small sizes of the monitor 770, external sensing units 571 are connected to it, to be applied to the chest, or to other part of the body, and according to another optional embodiment they allow sensing of more leads, for example of leads I and II, and further of all leads V1 to V6 and in an enlarged embodiment also of V1R to V6R and V7 to V9 by attaching of electrodes of the sensing unit 571 for a short time.

For a long term sensing, the sensing unit 571 in the form of a belt is connected to the monitor 770, which unit is attached to the body for a long time, and therefore, it makes possible to carry out continuous long term sensing of cardiac signals.

As a preferred embodiment, FIG. 33A shows a monitor 349 preferably formed by the monitor 770 with electrodes for attaching to the chest for sensing of cardiac signals and its fixing to the base 773 in the form of a small-plate with the help of the holder 774 and the connecting elements 446, preferably formed by spring contacts. The monitor 770, in this example of small sizes, approximately of a credit card, is preferably adapted to operate without any external reference electrode, preferably with an internally designed reference, and on it are located two sensing electrodes 143, preferably formed by electrodes 771 on the surface. Their relatively small spacing is suitable only for sensing cardiac signal from the immediate vicinity of the sternum, but it does not make possible to sense all chest leads Vx, and it does not make possible to sense against the Wilson's central terminal.

The monitor 770 is allocated for short-term sensing, when after attaching to the chest of the monitored person, preferably, the push-button 772 start is pressed for sensing, and preferably, another push-button 803 for transmission of the cardiac signals in the form of data to the cooperating unit 121. As the first example of the enlarged o use of this monitor also for short-term sensing of all chest leads Vx against the Kranz's central terminal, the base 773 in the form of a small-plate is shown, which is formed by base 420 of the universal monitor. The base solves direct interconnection of electrodes 771 on the surface of the monitor 770 with the holder 774 by the connecting elements 486, preferably formed by spring contact 714 on the base 773 with electrodes 652 and 653, which are parts of the base 420.

Preferably, the base 773 is formed by the base 420, which has greater spacing of electrodes 143 than the monitor 770, what makes possible to sense the leads Vx against the KS. In case, the base 773 in the shape of a small-plate, formed by the base 667 according to FIG. 16, makes possible connect external electrodes 143, preferably formed by electrodes 639, 660, 709 and the small-plates 448, 666, and 641 to the monitor 770, and to sense with the KS. In case, the base 773 is formed by the base 445 according to FIG. 6, it makes possible to sense with the WS. When the base 773 is formed by the base 667 according to FIG. 27A, it makes possible to sense against the KS or the WS switchable, and this for up to 21 leads.

The connecting elements 486, preferably formed by spring contact 814 allow connection of the monitor 770 directly to the electrode 421 of the base, instead of the original monitor, for example on the base 420 via the connecting field 711, for example in the base 445, so as it is shown in FIG. 9. In this case, the monitor 349 from the block diagram in FIGS. 9, 17, 27B, 28D is formed by monitor. To connect the monitor 770 to the mentioned bases only 2 connecting elements 486 are necessary, because the monitor 770 does not need in this measuring any external electrode for reference and it connects only one channel of the monitor to two inputs of the operational amplifier 426.

The view “D” shows the assembly of the monitor 770 and the base 773. The cardiac signal sensed by electrodes 652, 653 from the monitored person is applied by the contact to the spring contacts 814, which abut on the electrodes 771 on the surface of the monitor 770 inserted into the holders 774.

Detail 1 shows an embodiment in which the connecting element 486 is formed by spring contacts 814 on the base 773, preferably formed by the base 445 and connects the electrodes 771 of the monitor 770 with the electrodes 421 of the base with a conductor via a connecting field 711.

Detail 2 shows a variant, in which the connecting element 486 is formed by connector 775, contacting the monitor 770 with the base 773, preferably formed by the base 445, wherein, the electrodes 771 on the monitor are not used.

Relocation of the monitor 770 to the sensing unit in the form of a small-plate, for example on the base 773, preferably formed by the base 445 of the monitor for multi-lead sensing of ECG with a small-plate 448 shown in FIGS. 6 and 9 is possible to sense the leads I, II, calculate the leads a VF, aVR, aVL, and sense with the WS leads V1 to V6, V1R to V6R, and V7 to V9 by successive attaching of the small-plate 448 with the LL electrode 450 and the LA electrode 449, as it is shown in FIG. 13.

For calculation of the leads aVF, aVR, aVL sections of courses of leads I, II with the same lengths R-R are used. The block diagram shown in FIG. 9 for the monitor 349, which is formed in this case by the monitor 770, connected via the connecting element 486, shows how the mono-channel monitor 349 with the switches 454 and 453, located in the connecting field 711, is switched for sensing of all above-mentioned leads sensed with the help of relocation of the base 445.

In location of the monitor 770 on the base 773 formed by the base 667 with the reference, shown in FIG. 16 is able to sense the chest leads Vx against the KS with the reference electrode 644 according to the block diagram in FIG. 17 with monitor 349 formed by the monitor 770 instead of the monitor 668 using one operational amplifier 426, which is the only one comprised in the monitor 770, using the internal reference of the monitor 770.

During connecting of the monitor 770 to the base 773, preferably formed by the base 667, shown in FIG. 27A or 28A, it is possible via the connecting field 711 by successive relocation of the small-plats 666 for the reference electrode and by switching over of individual electrodes to the monitor 770 to sense up to 21 leads using the small-plate 654 for electrodes 639 either against the KS or the WS by switching over with the switch 700 KS/WS and only one operational amplifier 426.

For example for the KS the switch 700 is set to position 1 KS, the base 667 senses in the KS position 899, in FIG. 27C, when the internal pseudo-reference of the monitor 770 is used, the reference signal is not brought to the base 667 and the chest leads are sensed by the Vx/REF electrodes 638, wherein the switch 638 is set to the Vx position, or by the small-plate 654 with the electrode 639 when the switch 638 is set to the position 2 and relocation of the base to the KS position with the small-plate 900. For sensing using the KS, the base 667 is moved to the WS position 901, electrodes 194 RA and 195 LA are connected to the WS connector 701, see FIG. 27E, and the switch 700 is set to the WS position 2.

When using the reference electrode of the base 667, it is necessary to bring the reference signal from the monitor 667 via the connecting elements 486, place the base according to the selected sensing with appropriate setup of the switches according to the block diagram in FIG. 27B.

The base 773 fulfills the function of the sensing unit 571 of the adapter for the monitor 349, preferably formed by the monitor 770, and it can be connected to any monitor 770 for attaching by means of electrodes 771 on the case after the respective adaptation of the base 773 by adding of a connecting element, preferably formed by the spring contacts 814 for communication with the mono-channel monitor 770. It allows connecting of the monitor 349, preferably formed by the monitor 770 with connecting elements 486 to any base of a handheld ECG in the form of a small-plate.

FIG. 33B shows an example of transition from a short-term sensing by monitor 770 to a long term sensing by connecting a sensing unit 571 formed by a base 765 in the form of a belt with the holder 774 and of connecting elements 486, preferably formed by spring contacts 814 or connector 775 to the chest belt 749, preferably formed by the base 765 in the form of a belt. A monitor 349, preferably formed by the monitor 770, is preferably inserted from above into the holder 774, and connection of electrodes 771 with the holder 774 provide connecting elements 486, preferably formed by at least one from: a spring contact 814, snap fasteners 487, connectors 775. Connector 775 is shown as an example.

Interconnection of the electrodes 771 of the monitor 770 to the selected electrodes 143 with the base 765 in the form of a belt provides the holder 774 by connecting elements, preferably formed by the connector 775 with the electrodes 771 on the base 774, and further to belt 749 and by snap fasteners 487, and with the help of the connection 341 the connecting field 711. To the connecting field 711 can be brought also signals from the sensing unit 571 or from the external electrodes 143. This arrangement makes possible successive connecting of the required electrodes 143 through the connecting field 711 to the monitor 770 for sensing of up to 17 chosen leads. By selection of the base 765 types in the form of a belt, it is possible to sense with the KS or the WS up to 21 ECG leads. Preferably, the base 765 is formed by one of the bases 624, 620, 630.

When positioning the monitor 349, formed by the monitor 770, on the base 667, see FIG. 27A, using the small-plate 654 for the electrodes 639, it is possible to sense up to 21 leads either with the KS or the WS by switching with the KS/WS switch 700, when sensing with the KS and with only one operational amplifier 426.

For example, when sensing with the KS the switch 700 is set to position 1 of the KS, the base 667 with the reference senses in the KS position 899, in FIG. 27C, using internal pseudo-reference of the monitor 770, the reference signal is not brought to the base 667, and the chest leads are sensed by Vx/REF electrodes 638, wherein, the switch 638 is set to the position Vx or with the small-plate 654 with an electrode 639 when the switch 638 is re-set to the position 2 and the base is relocated to the KS position by the small-plate 900.

For sensing with the KS, e base 667 is moved to the position WS 901, electrodes 194 RA and 195 LA are connected to the WS connector 701, see FIG. 27E and the switch 700 is re-set to the position 2 of the WS,

When using the reference of the base 667, it is necessary to bring the reference signal from the monitor 667 through the connecting elements 486, place the base according to the choice of sensing with appropriate setup of the switches according to the block diagram, see FIG. 27B.

In case of location of the monitor 770 into the sensing unit 571 in the form of a belt, for example formed by the base 720, it is possible to sense with a mono-channel monitor 770 the leads I, II after switching of the switch 906 the I/II lead to the position B with switch 722 in the position A. After switching of the switch 722 to the position B, it is possible to sense the chest lead V4 and by switching of the V4/V4R/V8 switch 781 also to the V4R and V8.

Preferably, functionality time of the monitor 770 is prolonged with additional accumulator 120 located on the adapter 777 of the base and interconnected through the connecting field 711 with the monitor or with an inductive charger 776, which is preferably fastened in the adapter 777 directly to the monitor 770.

FIG. 33C shows another example from the short-term sensing by monitor 770 to the long-term sensing with the base 765 in the form of a belt, on which base is attached the monitor 770 for sensing, wherein, it is fastened with a connecting system 3006, which is formed by a holder 774, element the connecting elements 486 and by an connecting field 711, in this most simple case formed only by connection 982, which provides connection of electrodes 771 of the monitor with electrodes 143 of the base.

FIG. 33D shows another preferable embodiment for long term sensing by monitor 770 by using adapter 3007 for connection of the monitor 770 for attaching to the belt, which is designed for cooperation with the monitor, which is connected with connecting elements 486. Through these connecting elements is connected the holder 774 and into it is inserted the monitor 770 for attaching. Signals from electrodes 143 sensing unit 571 are brought via connection 982 through snap fasteners 487 and spring contacts 814 to the electrodes 143 of the monitor 770 for attaching.

FIG. 33E shows another preferable embodiment for a long term sensing with a connecting system 3006 in the design with an adapter 3007 making possible to connect a monitor 3008 with a different connector than what is the connector of the base 765, which is provided with the connector 775. With this connector it is inserted to the adapter 3007. The adapter 3007 is connected to the base 765 with connecting elements 486, preferably formed by snap fasteners 487.

FIG. 33F shows a general solution, in which the monitor 349 or the monitor 3008, provided with a different connector to the adapter 3007, is connected by a connecting system 3006 formed by connecting elements 486, and the adapter 3007 is connected to the sensing unit 571 with connecting elements 486′ to the other one.

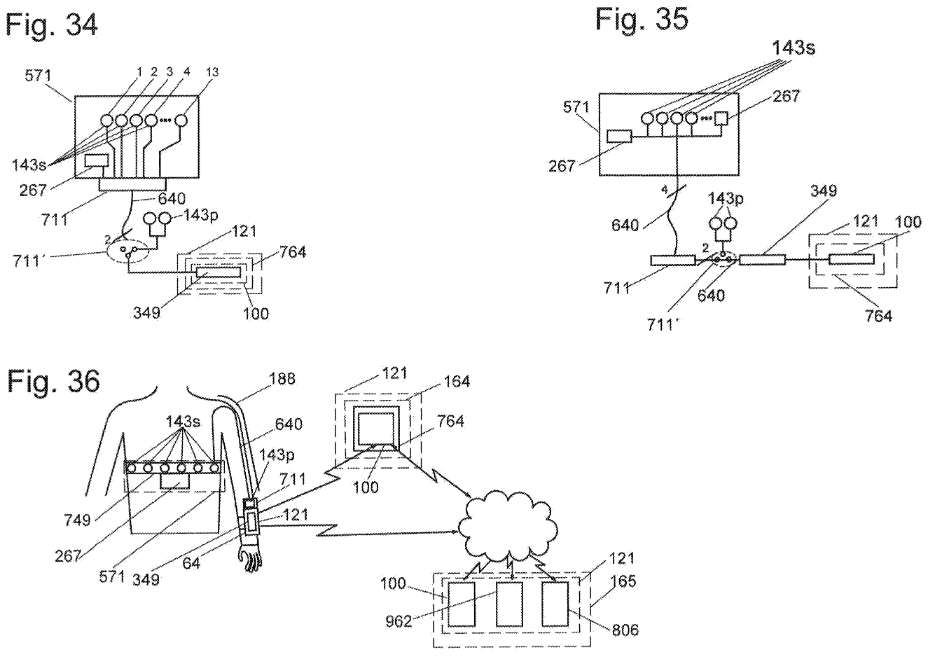

FIG. 34 shows a monitor 349 connected to the primary electrodes 143p for sensing of cardiac signals for a certain number of ECG leads, whose number can be increased by switching from, in this example two primary electrodes 143p, which are primary by means of a switch 711′, to a sensing unit 571, which comprises an optional number of sensing secondary electrodes 143s and/or circuits 267 by wires connected to the connecting field 711, located on or in the sensing unit, for a selection of electrodes 143s, wherein, the connecting field 711 is connected by an optional number of cores, in this example by a two-core cable 640, to the remote mono-channel monitor 349 with two inputs directly and/or through the selected circuits. In this example 13 electrodes 143s are chosen and the circuits 267, preferably for formation of the Wilson's central terminal, that allow to sense cardiac signals for up to 21 leads. It is optional how many electrodes 143s the sensing unit 571 comprises and how many channel monitor is used, from which it follows the number of electrodes 143p, which are primary, and how many cores has to have the cable 640 used. In this case, the monitor 349 is connected to the cooperating unit 121, preferably formed by the evaluating unit 764, which is preferably formed by mobile phone 100 or smart watch 416 for displaying and evaluation of the processed cardiac signals, preferably to ECG curves.

FIG. 35 shows an example of a mono-channel monitor 349, to which two electrodes 143p are connected, of which it is switchable with the switch 711′ to the sensing unit 571 through a remote connecting field 711. In this example a four-core cable 640 connects 4 secondary electrodes 143s, located in the sensing unit 571 to a remote connecting field 711, which connects two selected electrodes to the mono-channel monitor 349 by two conductors in the cable 640. The number of electrodes 143p, 143s, cores in cable 640, and the number of channels of the monitor 349 are optional.

FIG. 36 shows an example of location of a remote interconnecting field 711 located on a wrist of the monitored person or alternatively in/on the sensing unit 571, with which it is interconnected, formed in this example by a chest belt 749, multi-core cable 640 routed in a sleeve 188, or alternatively directly off the sleeve. Alternatively, the sensing unit 571 can be formed by a unit for attaching of an electrode, as it is shown I FIG. 7 or 28a. The interconnecting field 711 interconnects the selected electrodes 143s, located on the sensing unit 571 to the monitor 349, which is located on a wrist in the cooperating unit 121, preferably formed by the unit 764 evaluating, in this example represented by a smart watch 416, which is adapted for switching between sensing from the electrodes 143s to sensing from the electrodes 143p located on the cooperating unit 121 for sensing from a wrist and a finger of the other hand. Alternatively, the cooperating unit 121 is formed by a mobile phone 100. Optionally, the big connecting field 711 switches over successively to the respective electrodes 143 and/or the respective circuits 267 from the sensing units 571 to the monitor 349 to that will be possible to display the required number of ECG leads on the unit 764 successively. The circuits 267 are allocated for formation of a Wilson's or a Kranz's central terminals. It can be displayed, according to the accessory of the sensing unit 571, successively, for example 12 or up to 17 leads, and preferably, calculate four leads from the leads I. and II., from the actual courses of equal R-R spacing.

To each setup of the interconnecting field corresponds a display of the respective lead, (For measuring of individual leads, the connecting field 711 and the course of the signal of the selected lead are adjusted respectively), which is preferably displayed on the displaying and evaluating unit 764, preferably formed by a smart watch 416 and/or it is saved in memory, and later on it is switched over through the connecting field 711 to the sensing from further electrodes 143 for displaying or saving of the course of another lead. After saving in memory of the course of the last chosen lead, it is, therefore, possible to display all leads on the cooperating unit 121, preferably formed by the evaluating unit 764, preferably formed by a smart watch 416, successively or at once from the memory, or the data can be transmitted to a near cooperating unit 164, formed by the evaluating unit 764, preferably provided with a larger display than what is that of the smart watch 416, preferably formed by a mobile phone 100 and/or a PC 962 and/or, preferably, to transmit the data to a remote cooperating unit 165, preferably formed by a remote PC 962, a server 806, or a remote mobile phone 100 of the chosen participants.

Connection of the evaluating unit 764 for a wrist with the nearby cooperating units 164 is realized with wired connections or local wireless connection, connection with the remote cooperating units 165 is realized with wireless connections, mobile network or long-distance wireless connections.

For permanent monitoring, permanent switching is selected or interconnection of the appropriate electrodes 143 and/or circuits 267 through a connecting field 711 to the monitor 349 for displaying in the displaying units 764 and/or cooperating units 121. The interconnecting field 711 is adapted so that it can continuous monitoring mode temporarily switch to the selected electrodes 143 and/or circuits 267 for timely monitoring of the selected leads, and tis manually, locally, or remotely, and/or automatically in optional time periods. Thereby switch to other selected electrodes 143s and/or circuits 267 for sensing of other leads.

As the sensing unit, any unit described in this application can be selected, for example those shown in FIGS. 6, 8, 9, 31, etc.

FIG. 37 shows an example of a mono-channel monitor 349, which is located in the cooperating unit 121, formed by the evaluating unit 764, formed by a smart watch 416, in this example it is the “Apple watch”, for sensing of one ECG lead, and this of the lead I, by sensing from the electrodes 143, which are located on the smart watch 416, formed by electrodes 143p, which are the primary ones. For sensing of more leads, the monitor is the monitor 349, which is reconnected from the sensing from the electrodes 143p, which are the primary ones, to sensing from the secondary electrodes 143s, which are located in the sensing unit 571, using the following method.

For sensing of the signal from one hand, for example from the left side, the smart watch 416 has one primary electrode 143p, formed by electrode 143″ for sensing from the wrist, which is located at the bottom of the watch 416, to which the spring contact 814 is connected to the connecting field 711 on the bases 259 of the interconnecting field 711, which is located between the wrist and the smart watch 416. The other primary electrode 143p, preferably formed by the electrode 143′ for sensing from the other hand, is formed by the crown 223 of the watch, and it is connected via the other contact 258 of the base, shown in the pushed away position, which in the pushed in position of the movable part 259′ of the base, which is located on the base 259, rests on the crown 223 so as it is shown in Det. 1. The base contact 396 connects the movable part 259′ of the base to the base 259, whereby, the other contact 258 of the base is connected electrically to the base 259, and further to the connecting field 711. The Electrodes 143′, 143″ are connected to the sensing unit 571 with the interconnection 395 via the connecting field 711 for sensing from electrodes 143s.

The movable part 259′of the base can be moved away from the crown 223, as it is indicated in view “D2”. In this removed position, it is possible to control the crown of the smart watch 416. The base 259 isolates the electrode 143″ located on the underside of the smart watch from the wrist while sensing signal the cardiac signals from the secondary electrodes 143s of the sensing unit 571, which is connected to the connecting field 711. After removal of the base 259 with the connecting field 711 from underneath the smart watch 416, in this example the “Apple watch”, the smart watch 416 is ready to function again by sensing with the electrode 143″ applied to the wrist and the electrode 143′formed by the crown 223, on which a finger is applied. The monitor 349 located in the smart watch 416, is preferably connected to the sensing unit 571 based on attaching of electrodes 143, preferably formed by hand-held electrodes 143, shown for example in FIGS. 6, 9.

The interconnecting field 711 connect the relocateable electrode 143m and/or successively individual sets of electrodes 143s to the monitor 349 for successive sensing of cardiac signals up to 21 ECG leads. In the sensing units 571 based on principle of a belt, shown for example in FIGS. 18A, 22, 31, the connecting field 711 interconnects to the monitor 349 successively always two electrodes 143 formed by the electrodes 143s or by circuits 267 for sensing of one ECG lead, optionally, of one of the leads I, II, V1 to V6, V1R to V6R, V7 to V9 and 4 leads can be calculated. Therefore, it is possible to choose displaying of up to 21 ECG leads. The interconnecting field 711 is controlled by the control elements 684 and optionally it is located on the base 259, or in/on the sensing unit 571.

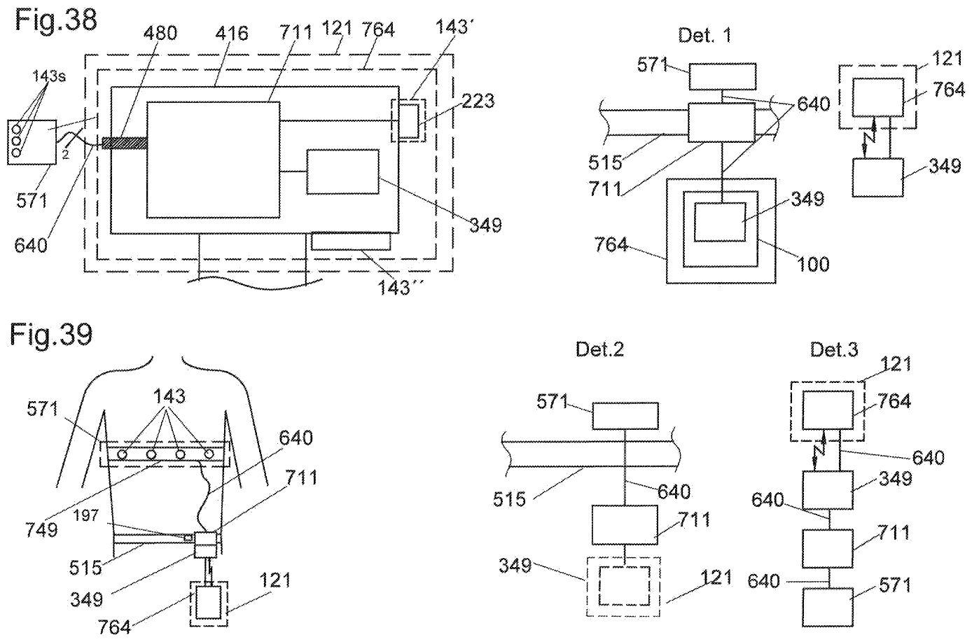

FIG. 38 shows a sensing unit 571 with electrodes 143s connected by a cable 640 via a connector 480 to a connecting field 771 located in the cooperating unit 121 preferably formed by the evaluating unit 764, preferably formed by a smart watch 416. Electrodes 143p, and this the electrode 143″ for a wrist, preferably formed by a spot on the watch 416, and electrode 143″, preferably placed on the crown 223, are during the time period signal of sensing the cardiac signals from the sensing unit 571 disconnected by the connecting field 711, which instead of them connects the sensing unit 571 for sensing from electrodes 143s. The interconnecting field 711 connects back to the electrodes 143p for sensing from arms for completing the sensing from the sensing unit 571.

FIG. 39 shows connection of the sensing unit 571 with electrodes 143s, preferably formed by chest belt 749, with the help of a cable 640 to the connecting field 711, preferably located on the waist belt 515 in the waist of the monitored person, where it is preferably located also the electrode 197 LL. Further, the interconnecting field 711 is connected to the monitor 349, preferably also located on the belt 515, and from this belt by wires or wirelessly to the cooperating unit 121, preferably formed by the evaluating unit 764 located for example on the trausers waist belt, preferably hung on a buckle, or in a pocket.

Or is connecting field 711 is connected by wires to the monitor 349 located remotely from the interconnecting field 711, preferably on or in the evaluating and displaying unit 764, preferably formed by a mobile phone 100, with which the sensing unit 571 is connected by a cable 640 via the connecting field 711, as it is shown in Det. 1.

Or, the monitor 349 is remote from the cooperating unit 121, preferably formed by an evaluating and displaying unit 764, and to which it is connected by wires or wirelessly.

Or, as it is shown in Det. 2, the connecting field 711 is located remotely from the sensing unit 571 and it is interconnected by a cable 640 to the sensing unit 571 and preferably it is located on or in the 349, as it is shown in dashed lines.

Det. 3 shows a configuration of a sensing unit 571, with wires connected by a cable 640 to the connecting field 711, and further to the monitor 349, which is preferably located together with the connecting field 711 to/in the sensing unit 571. To the evaluating and displaying unit 764 it is connected by wires or wirelessly.

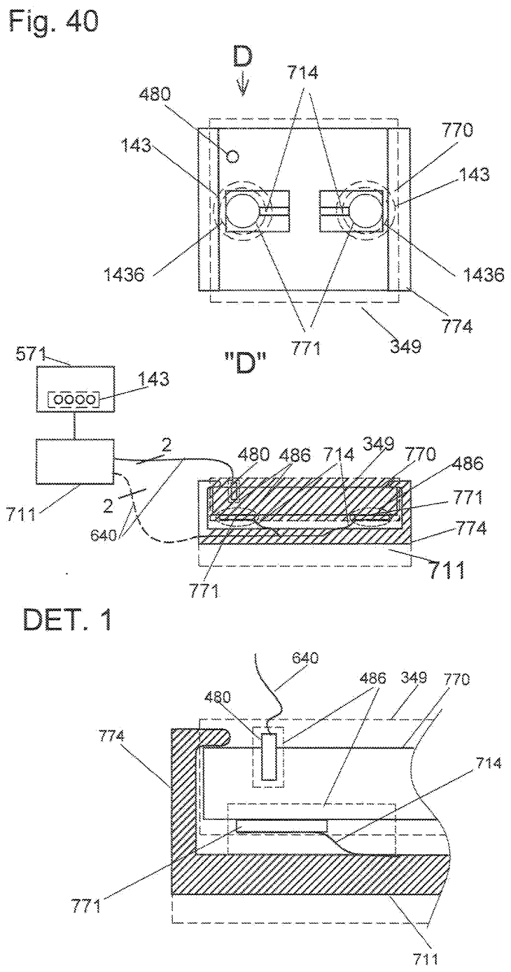

FIG. 40 shows connection of the interconnecting field 711 by a cable 640 to the monitor 349 by connecting elements 486, preferably formed by connector 480 for connection or the connecting field is connected to the holder 774, as it is shown with dashed lines, preferably interconnected with spring contacts 814 to the electrodes 771 on the surface of the monitor 349.

FIG. 41 shows a preferable use of the sensing unit 571 in a combination with up to 12 lead ECG device 19, wherein, instead of electrodes 143, preferably formed by electrodes 143p, which will be disconnected, with the help of the connecting elements 486, preferably formed by connector 480, electrodes 143 for sensing, in this example of up to 21 lead ECG, formed by electrodes 143s, will be connected to its inputs through connecting field 711. For such sensing, the sensing unit 571 according to the invention provides all necessary signals from electrodes 143s, which are connected through the connecting field 711. All or great majority of electrodes 143s of the sensing unit 571, preferably formed by the chest belt 749, preferably formed by multi-electrode base 630, is located on the chest belt 749, and therefore, such installation of the sensing elements on a patient is much faster on the patient and more comfortable than how it is in case, for example, of individual glued suction-fixed or clipped electrodes that are used in the present state of the art.

The interconnecting field 711 is connected by a cable 640 drawn with solid line and it is located externally to the sensing unit 571 near the ECG device 19 for easy control of switches 684 of the interconnecting field using the controlling elements 795 of the interconnecting field.

Alternatively, the interconnecting field 711 is located on chest belt 749, and it is connected by a cable 640 drawn in dashed lines.

For the first 12-lead ECG the connecting field 711 is switched for interconnection of the appropriate electrodes 143s of the first set of the sensing unit 571 to input of the ECG device 807 for sensing of leads I, II, V1 to V6 and four leads are calculated. For sensing of the right-side V1R to V6R and the back leads V7 to V9, the connecting field 711 is switched from electrodes 14453s of the first set to the electrodes 143s of the second set on the sensing unit 571 for these leads.

The signals of all electrodes 143s of the sensing unit 571 are fed to the input of the interconnecting field 771, preferably for sensing of cardiac signals for 21 up to 21 leads. Preferably, all 10 cables of the ECG device 19a are connected to the outputs of the interconnecting field 711. The interconnecting field 711 makes possible to switch the electrodes 143s to the ECG cables for sensing of cardiac signals for a combination of leads, the physician defines for himself. The maximum number 21 of leads is for the twelve-lead ECG device 807, available in two events of sensing with one switching over.

When a ECG device 19 is used, which measures less than 12 leads, to obtain 21 leads, it is necessary to make the switching-over several times.

Preferably, for the sensing unit is used the multi-channel base 630 shown in FIG. 31, preferably, with the Kranz's central terminal 692 and the electrode 194 RA′ and 195 LA′ on a belt to form pseudo-the Wilson's central terminal, and therefore, all electrodes 143 are located on the chest belt. The multi-electrode base 630 is adapted for swapping of the KS electrodes for the LL ones and of LA′, RA′ electrodes for LA, RA electrodes, located off the belt for greater accuracy of sensing, for example for electrodes 143, preferably formed by bulky electrodes 702, for the electrode 197 LL on abdominal small-plate 721 and for another ones.

FIG. 42A shows various possible locations of electrodes 143p and of the monitor 349 of cardiac signals for sensing from the chest or from the fingers, which is provided on its surface with primary electrodes 143p. The monitor 349 is provided with communication circuits 368 through which it transmits information on the external processed cardiac signals to the cooperating unit 121, in particular for their displaying.

FIG. 42B shows situation when the monitor 349 is located in the cooperating unit 121 and the primary electrodes 143p for sensing of cardiac signals are located on the surface of the cooperating unit 121.

FIG. 42C shows a monitor 349 with electrodes 143p and a cooperating unit 121, which are located externally, off the monitor 349.

FIG. 42D shows a monitor 349 located in the cooperating unit 121 with primary electrodes 143p, which are located externally.

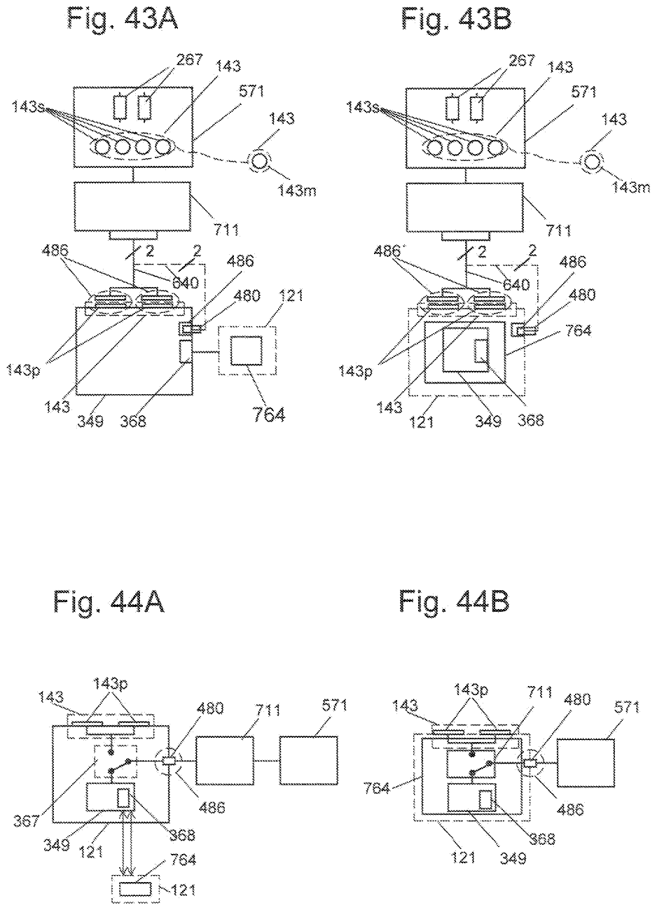

FIG. 43A shows the connection of the sensing unit 571 to the monitor 349, shown in FIG. 32A through the interconnecting field 711 and the connecting elements 486, and switching from sensing from the electrodes 143p, which are connected to the monitor 349, to the sensing with the secondary electrodes 143s, which are located in the sensing unit 571.

The connecting elements 486 ensure the transmission of cardiac signals to the primary electrodes 143p of the monitor from electrodes 143s of the sensing unit 571, located in the unit 571 or from the relocateable electrodes 143m. The signals are fed to connecting field 711 directly or via the circuits 267, which form modified signals, for example for the Wilson's central terminal 428 or for the electrode 432 of the Kranz's central terminal or for their variants, such as the strengthened Kranz's central terminal 692 or the pseudo-Kranz's central terminal 693.

Operator of the equipment selects the method of switching over of electrodes 143s for individual ECG leads for successive processing. The sensing unit 571 is interconnected with the connecting field 711 with so many conductors, how many electrodes 143, and/or circuits 267 is necessary to connect with the monitor 349. Preferably, the connecting elements 486 are formed by spots, that are connected to spots of electrodes 143p for to the aiming of a contact.

Alternatively, the signals from the sensing unit 571 are fed by a cable 640, shown as dashed line, to the monitor not through primary electrodes 143p, but through connecting elements 486, preferably formed by connector 480.

FIG. 43B shows connection of the sensing unit 571 via the connecting field 711 to the monitor 349, which is located in the cooperating unit 121. The monitor communicates with the cooperating unit 121, in which it is located, or with external cooperating units via the communication circuits 368.

FIG. 44A shows function of the switching elements 367 for switching over of the electrodes 143p to the electrodes 143s of the sensing unit 571 through the connecting field 711 to the monitor (349) located in the cooperating unit 121 through the connecting elements 486, preferably formed by a connector 480, to the monitor 349 located in the cooperating unit 121. The switching elements 367 switch from sensing by means of electrodes 143p located on the surface of the cooperating unit 121 to the sensing by means of electrodes 143s of the sensing unit 571. During a switching to the sensing from the sensing unit 571, at the same time, the switching elements 367 disconnect the monitor 349 from the primary electrodes 143p. The electrodes 143p are located on the cooperating unit 121, and the switching elements 367 are located in the cooperating unit 121.

FIG. 44 shows an independent monitor 349, which is located separately from the cooperating unit 121, and the switching elements 367 are located in the monitor 349, and the primary electrodes 143p on the monitor 349. The monitor 349 communicates with the external cooperating unit 121 by means of the communication circuits 368. The interconnecting field 711 is located in the cooperating unit 121. In such case the connecting field 711 may take over also the function of the switching elements 367, and therefore, to disconnect the primary electrodes 143p from the monitor and to connect the sensing unit 571. In this case, so many conductors are led via the connector 480, how many electrodes 143 and outputs from the circuits 267 the sensing unit 571 comprises.

The monitor 349 is independent, and the connecting field 711 is located externally, and the primary electrodes 143p are on the monitor 349. The monitor 349 communicates with the external cooperating unit 121 with the help of the communication modules 368 through wires or wirelessly.

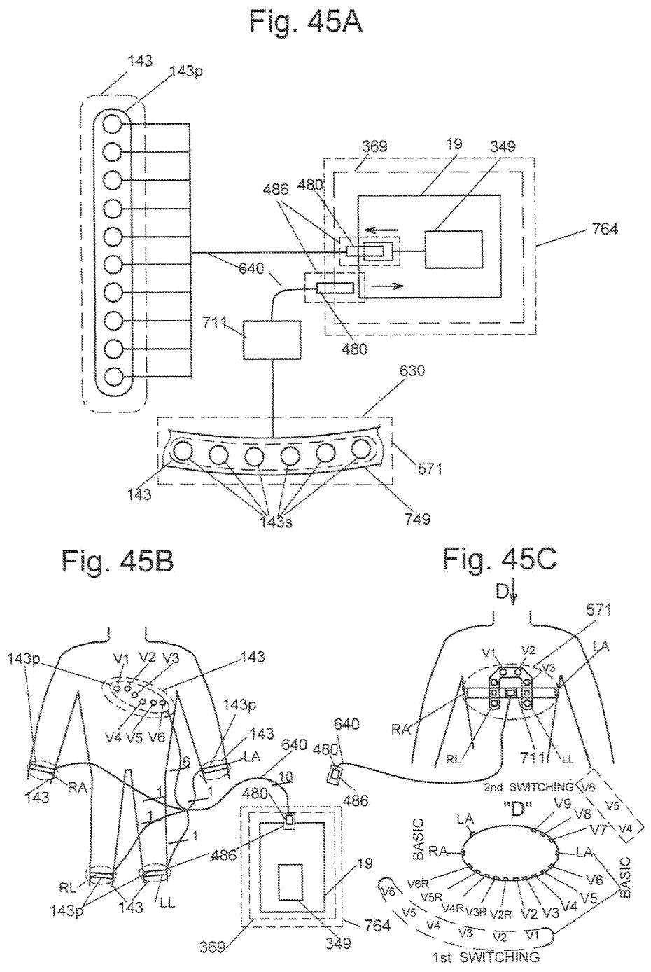

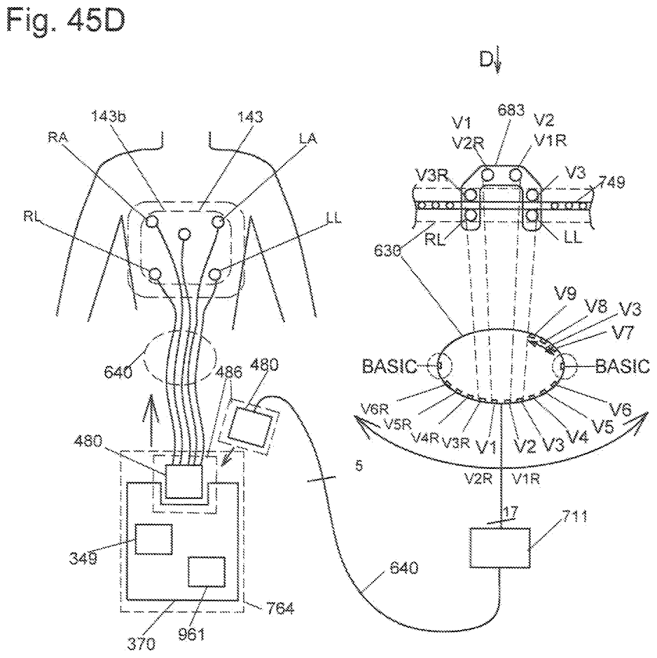

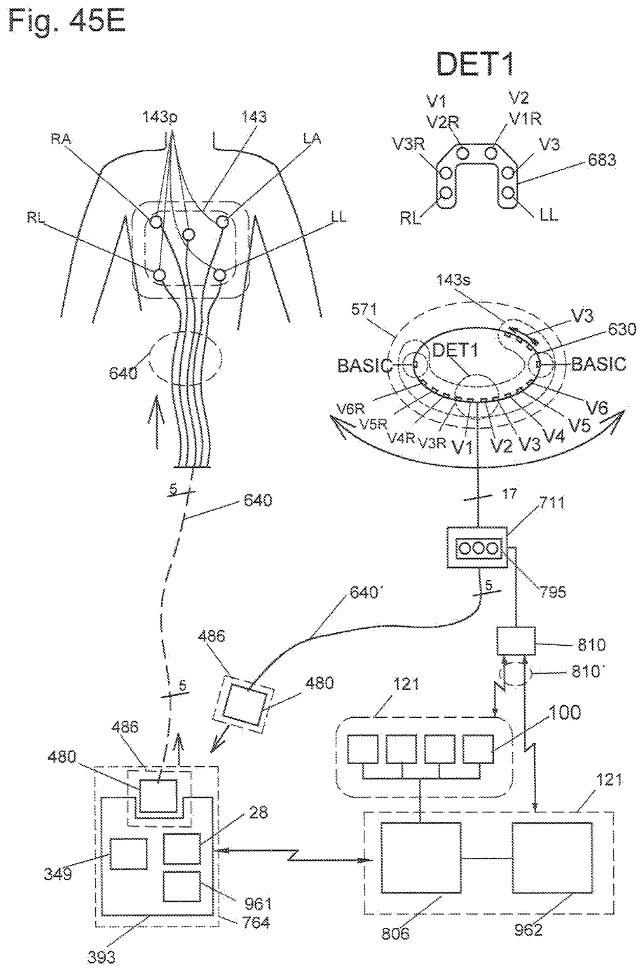

FIG. 45A shows primary electrodes 143p which are located externally to the ECG device 369 with external electrodes preferably formed by the ECG 19 or by a monitor for monitoring of patients on bed in hospital facilities. The electrodes 143p are connected to the device 369 by connecting elements 486, which are preferably formed by the connector 480, and they allow the basic sensing of cardiac signals for a certain number of leads.

For the enlarged sensing of cardiac signals for more leads, than what allow the primary electrodes 143p for the basic sensing of cardiac signals, allowing to sense for example from 2 to 12 leads, the primary electrodes 143p are disconnected by disconnecting the connecting elements 486, for example by pulling out the connector 480 from the ECG device 369 and by connecting with another connector 480 the sensing unit 571, which is provided with a greater number of secondary electrodes 143s than what is the number of primary electrodes 143p, which allows to sense cardiac signals for more leads than what allow the electrodes 143p, and this for up to 21 leads, by successive interconnecting of the electrodes 143s, which are located in the sensing unit 571, through the connecting field 711 to the monitor 349 located in the ECG device 369.

The device 369 for short-term static sensing, as for example the ECG device 19, processes, at first, the cardiac signals from the first connected set of secondary electrodes 143s of the sensing unit 571 connected through the connecting field 711, displays them and/or saves them in memory 691, and thereafter, it processes the signals from another set or sets of electrodes 143s and displays and/or saves data in the memory 691.

After processing and saving in memory of the cardiac signals from all sets of electrodes 143, preferably their record is displayed and/or printed, i.e. of data from the sets of electrodes successively or of all sets at once, preferably of up to 21 ECG leads stored in memory, or only the selected leads. For a long term live sensing, on the device 369, for example on the monitor for monitoring of patients on bed, up to 21 leads will be selected, which the device 369 can display by means of the electrodes 143s as the required leads for simultaneous displaying, and they are switched manually, automatically, or remotely to further leads to be displayed as needed.

FIG. 45B shows the standard connection of the ECG device 369 with external electrodes, formed by 12-lead ECG device 19 with 10 electrodes 143 for sensing formed by primary electrodes 143p, formed usually by clips for sensing of signals from RL, LL, RA, LA on limbs and by suction pads in places for sensing of chest signals V1 to V6, which are connected by a cable 640 and by connecting elements 486, preferably formed by connector 480 into the ECG device 19.

FIG. 45C shows, how by disconnecting of the connecting element 486, formed by the connector 480, which is interconnected to a device 369 formed by the 12-lead ECG device 10 electrodes 143p are disconnected from it, and by another connector 480 the sensing unit 571 with secondary electrodes 143s is connected, which is formed preferably by the multi-electrode base 630, which in the basic connection through the connecting field 711 connects the ECG 19 with monitor 349 to the first set of electrodes 143s, preferably for sensing of the same leads that were sensed by ten primary electrodes 143p, preferably 12 ECG leads, that is by leads I, II and V1 to V6.

Preferably, all or most of the electrodes are located on the multi-electrode 630 base in the form of a belt. Including the limb electrodes 143 for LA, RA, RL, and LL it is derived from RA, LAj4 and their location is shown in FIG. 31. During the first switching over to the second set of electrodes, instead of the leads V1 to V6, the leads V1R to VOR are sensed, and during the second switching over to the third set of electrodes, the leads V7 to V9 are sensed. Preferably, the limb electrodes LA, RA, LL, RL se are not switched over, and the electrode 143s for RL serves for reference, and the electrodes 143s for LA, RA, LL serve for formation of the Wilson's or the Kranz's central terminals, or of their variants.

Alternatively, the electrode LL of the multi-electrode base 630 is located under the waist belt, lower to the left in the abdominal area, as it is shown in FIG. 31 with dashed lines, where the cardiac signal is recordable with greater accuracy, and the electrodes for leads LA, RA are situated off the base so as it is shown for example in FIGS. 22, 24C, 24D.

The FIG. 45D shows a device 369 formed by Holter 370 with five electrodes 143, preferably formed by primary electrodes 143p, which are located externally, preferably glued, connected by a cable 640 and by a connecting element 486 preferably formed by connector 480 to a Holter 370. The electrodes 143p are designed for the limbs LA, RA, RL, LL and for one chest lead.

The monitor 349 in the Holter 370 processes the cardiac signals from these electrodes 143p for up to 7 leads, wherein it senses the I, II, V3 and calculates four leads III, aVF, aVR, aVL. For successive processing of up to 21 leads the primary electrodes 143 are disconnected by disconnecting the connecting element 486, preferably, by pulling the connector 480 out, and connected is the sensing unit 571, provided with electrodes 143s, preferably formed by multi-electrode base 630, shown in FIG. 31, via the connecting field 711 by inserting the connector 480. The monitor 349 of the base 630 is not connected and it is used the monitor 349 located in the Holter 370.

In the first connection of the connecting field 711 to the Holter 370, the electrodes 143s of the sensing unit 571 are connected for sensing of the same leads, which have sensed the primary electrodes 143p of the Holter 370, that is the RA, LA, RL, LL and one chest lead, for example the V3.

In the next switching, the connecting field 711 on the input of the Holter 370 switches successively the electrodes 143s of the sensing unit 571, determined for sensing of the V1, V2, V4, V5, V6, V3R-V6R, V7-V8, that is together 15 switches to the input amplifier of the monitor 349 of the Holter, originally determined for example for V3. After each switching of electrodes, the data from the optional period of time for sensing is saved in memory 961 of the Holter for later evaluation.

The switching over of the electrodes 143s for the chest leads Vx is preferably electronic, preferably it is carried out automatically, so that the Holter senses, preferably within 24 hours successively in the optional time periods all chest leads V1-V6, V1R-V6R, V7-V9 successively, and the leads I and II permanently, wherein, from them the leads III, aVR, aVL, aVF can be calculated.

Alternatively, for processing of the chest leads are used another two inputs of the monitor 349 to the amplifier for chest lead, which inputs were allocated originally for leady I and II for processing of chest leads, what allows switching over of three chest electrodes 143s of the sensing unit 571 to three amplifiers simultaneously, so that the lower number of 5 items of switching manage all 13 chest leads V1 to V6, V1R to V6R, V7 to V9.

In case, the Holter 370 for the primary sensing is adapted for sensing with more input circuits of more chest leads at once, than the above described one lead, the number of switchings will be proportionally lower.

In FIG. 45E, the common connection of the primary electrodes 143p to the monitor 393 at a hospital bed for ECG monitoring of a patient is shown in dashed lines, with five-core cable 640 on the connecting element 486, preferably formed by the connector 480, for sensing of up to 7 leads. For sensing of up to 21 leads, the monitor is switched to the electrodes 143s of the sensing unit 571 by disconnecting the connector 480 and by connecting the other connector 480′.

The sensing unit 571, preferably formed by the multi-electrode base 630, wherein, the monitor 349 is located in the monitor 393 at a hospital bed. The function of the monitor 393 with the sensing unit 571 is similar to that of the Holter 370, but the monitor 393 displays ECG on display 28 of the evaluating unit 764 live. The monitor 349 is connected to the cooperating unit 121, preferably the server 806 and the PC 962 for displaying in the monitoring room of the health care personnel and on mobile phones 100 for participants for a long-distance monitoring of patients connected to the monitor 393.

The electrodes 143s are connected to the monitor 393 via the connecting field 711 using manual switches or electronically, preferably remotely with the help of a long-distance control 810, which is connected with the help of the long-distance connections 810′, so that the health care personnel can make a choice which leads to follow remotely. Preferably, the interconnecting field 711 is adapted to switch over the sets of electrodes of the monitor 349 automatically according to a pre-programmed time schedule, so that the health care personnel can follow and evaluate successively all leads live or from the memory without any other setup. Thereby, the monitor 393, which is originally adapted for processing of a certain number of leads, in this example of seven leads, able to process a higher number of leads, up to 21, and display them live successively, or to save them in memory 967 for future displaying.

Det. 1 shows a bridge 683, which can be fastened to the multi-electrode base 630 for location of up to six ECG electrodes 143 from the base 630 into a more advantageous position for sensing of cardiac signals.

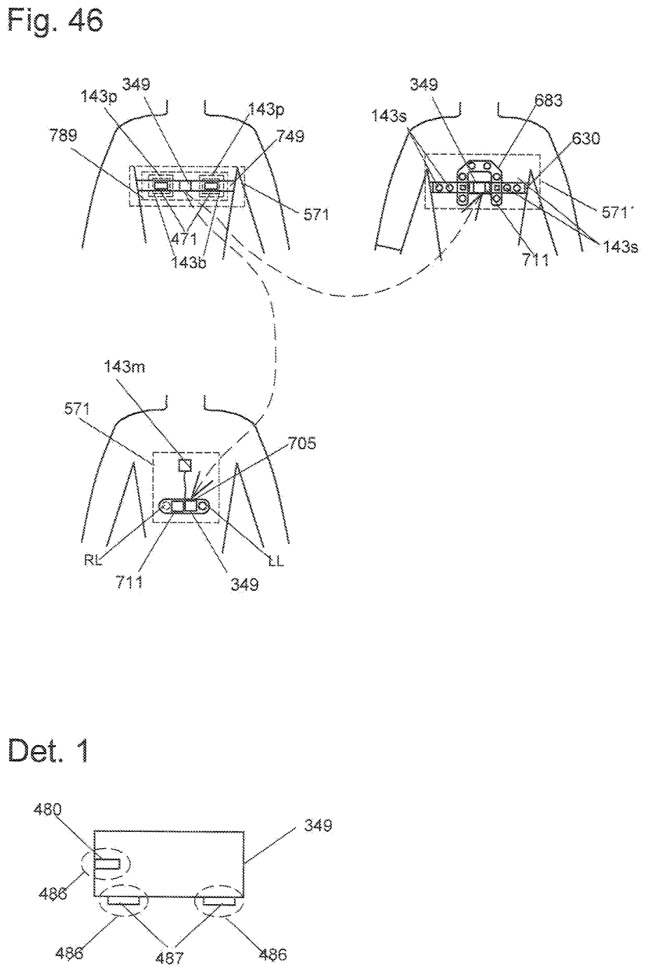

FIG. 46 shows switching of the monitor 349 from sensing of the primary electrodes 143p, to sensing of the secondary electrodes 143s of the sensing unit 571 by relocation of the monitor from one sensing unit 571 to another sensing unit 571′ indicated with the help of the connecting elements 486, in this example formed by snap fasteners 487 or by a connector 480. The relocation of the monitor allows for example to sense the cardiac signals permanently using the monitor situated on a simple base on the chest belt 789 and to move the monitor to the base 705 for the LL, RR with a relocateable electrode 143m or to the base 639, which allow to sense the cardiac signals of more leads for processing by the monitor 349, than what makes possible the base 143p with electrodes, onto which the monitor was placed before the relocation. This allows to use simpler bases that are more easy to implement, when the more complex bases are not necessary and to move the monitor 349 to a more powerful sensing unit 571 in case of need.

Detail 1 shows a detailed view of the monitor 349 with connecting elements 486, preferably formed by the connector 480 and/or by snap fasteners 487 for connection and/or fixing of the monitor 349 during relocation.

### Industrial Use

This equipment is usable in the border range between the consumer and medical fields of technology.

1. An equipment for increasing of the number of leads for the ECG devices, characterized in that it comprises:
at least two primary electrodes (143p) for the primary sensing of analog cardiac signals for their processing to digital data for at least one ECG lead by a monitor (349), wherein their maximum number is given by the number of input circuits of the monitor (349);
a sensing unit (571) comprising secondary electrodes (143s) for secondary sensing of analog cardiac signals for their processing by the monitor (349) to digital data for more leads or for sensing of other types of leads than what are those which allow to sense the primary electrodes (143p), and this for sensing of one of the chest leads, for a long-term continuous recording, and/or, the sensing unit (571) comprises circuits (267) for connecting elements (486) for connecting of the secondary electrodes (143s) directly or through a switching field (711) to the primary electrodes (143p), or the switching elements (367) for switching over of the monitor (349) directly or via the connecting field (711) from the primary electrodes (143p) to the secondary electrodes (143s), which are located in the sensing unit (571);
a monitor (349) for processing of analog cardiac signals sensed at least from two primary electrodes (143p) for processing to digital data for displaying of at least one ECG lead, which is adapted for sensing from the secondary electrodes (143s), which are located in the sensing unit (571), by switching over from the primary electrodes (143p) to the secondary electrodes (143s) or by connecting of the secondary electrodes (143s) to the primary electrodes (143p) for successive processing of more ECG leads than what is possible from the electrodes (143p).

2. The equipment for increasing of the number of leads for the ECG devices according to claim 1, characterized in that it comprises:
a memory (961) for storing of digital data, processed by monitor (349) from the analog cardiac signals sensed by the primary electrodes (143p)and/or by the secondary electrodes (143s), which are located in the sensing unit (571) for subsequent evaluation and/or for displaying of the ECG signals and for data transmission, what is carried out by relocation of the memory (961) to the cooperating unit (121) for transmission of the saved data to the cooperating unit (121);
or the data transmission is carried out with communication circuits (368) for wire or wireless transmission of digital data from the memory (961) or live from the monitor (349) to at least one cooperating unit (121), in which or on which the monitor (349) is located, or to the cooperating unit (121), which is located remotely from the monitor (349).

3. The equipment for increasing of the number of leads for the ECG devices according to claim 1, characterized in that it comprises:
at least one cooperating unit (121) located remotely with regard to the monitor (349), or a cooperating unit (121), in which or on which the monitor (349) is located, which is adapted for displaying and/or evaluation of the ECG curves from the digital data transmitted via the communication circuit (368) live or from the memory (961) or from the data transmitted from the relocated memory (961) to the cooperating unit (121), which is adapted for successive or simultaneous displaying of the optional ECG leads.

4. The equipment for increasing of the number of leads for the ECG devices according to claim 1, characterized in that
the primary electrodes (143p) are located on one of the following:
the monitor (349);
the cooperating unit (121), in which, or on which the monitor is located;
the cooperating unit (121) located remotely with regard to the monitor (349) and connected by a cable (640) and by a connecting element (486) to the monitor (349).

5. The equipment for increasing of the number of leads for the ECG devices according to claim 1, characterized in that
further, it comprises a connecting field (711), which successively connects the sets of electrodes (143s) and/or the circuits (267) of the sensing unit (571) to the monitor (349) to increase the number of the ECG leads, which can be obtained from the cardiac signals sensed with the electrodes (143s) in comparison to the ECG leads, which can be obtained by sensing from the electrodes (143p),
wherein, the increasing is reached by successive sensing of cardiac signals from the successively connected optional secondary electrodes (143) via the connecting field (711) or of optional sets of electrodes (143s) and/or of circuits (267), which are located in the sensing unit (571) for successive processing by the monitor (349).

6. The equipment for increasing of the number of leads for the ECG devices according to claim 1, characterized in that
the sensing unit (571) comprises at least one relocateable electrode (143m), for successive relocation for its application to the spots designated for sensing of cardiac signals from the chest leads for their processing by the monitor (349) to digital data for displaying of the chest ECG leads.

7. The equipment for increasing of the number of leads for the ECG devices according to claim 1, characterized in that
the monitor (349) is connected to the electrodes (143p) located on the monitor (349) or on the cooperating unit (121), in which the monitor (349) is located, which allow one-time sensing of cardiac signals by attaching of the electrodes (143p) to the chest, or by attaching of fingers of both hands to the electrodes (143p),
and this of a finger of the left hand to the electrode (143p), which is designated for the left hand and of a finger of the right hand to the electrode (143p), which is designated for the right hand for sensing of cardiac signals for one ECG lead, and by changing-over or by connecting of the electrodes (143p) to the electrodes (143s), which are located in the sensing unit (571)', sensing of more or leads of other leads is obtained, than what allow the primary electrodes (143p), optionally during a short-term or a long-term sensing.

8. The equipment for increasing of the number of leads for the ECG devices according to claim 1, characterized in that
the monitor (349), which is located independently or on the cooperating unit (121), is adapted for one-time or short-term sensing from the primary electrodes (143p), which are distant from the monitor (349), and which are connected to the monitor (349) by a cable (640) and by connecting elements (486),
and/or from the electrodes (143p), which are located on the monitor (349), or on the cooperating unit (121), in which the monitor is located, wherein, the electrodes (143p) are allocated for short-term attaching to the body of a patient, to the chest, hands or fingers, and the monitor is adaptable for the long-term or continuous monitoring by disconnecting of the connecting element (486) and by connecting of the other connecting element (486) via a cable (640) connecting to the electrodes (143s), which are located in the sensing unit (571) for the long-term sensing, wherein, preferably, the sensing unit (571) is formed by a sensing unit in the form of a belt, preferably by a multi-electrode base (630).

9. The equipment for increasing of the number of leads for the ECG devices according to claim 1, characterized in that
the monitor (349) is adapted for relocating with connecting elements (486) between the base with primary electrodes (143p) and the sensing units (571), and for sensing of cardiac signals by a higher number and/or by a different type of the electrodes (143s), what allows processing of the cardiac signals by the monitor (349) into a greater number, or to different ECG leads, than what would allow the primary electrodes (143p),
or in case of the monitor (349), which is located on the base with primary electrodes (143p), which are adapted only for the one-time or the short-term testing by attaching of the primary electrodes (143p), to the body, by relocation of the monitor (349) from the base to the sensing unit (571) with electrodes (143s), preferably in the form of a belt, it makes possible to carry out the long-term or the continuous sensing.

10. The equipment for increasing of the number of leads for the ECG devices according to claim 1, characterized in that
the memory (961) is adapted for successive storing of the digital data that is processed successively by the monitor from the analog cardiac signals, which signals are sensed from the electrodes (143s), which are located in the sensing unit (571) connected successively to the monitor (349) via the connecting field (711).

11. The equipment for increasing of the number of leads for the BCG devices according to claim 1, characterized in that
the circuits (267) are formed by one of the following:
circuits (372) for forming of a Wilson's central terminal, preferably with resistors (425);
a module (805) for sensing of the chest leads with Kranz's central terminal, preferably formed by an electrode (433) for the Kranz's central terminal;
an electrode (804) for the chest leads;
circuits (391) for a strengthened Kranz's central terminal, preferably formed by three resistors (425), which are connected to RA, LA Kranz's central terminal;
circuits (392) for a pseudo-Kranz's central terminal, preferably formed by two resistors;
circuits (393) for a pseudo-Wilson's central terminal, preferably formed by resistors (425), which are connected to RA, LA, LL.

12. The equipment for increasing of the number of leads for the ECG devices according to claim 1, characterized in that
the connecting field (711) is adapted for disconnecting of the primary electrodes (143p) from the monitor (349) and for connecting of a sensing unit (571) to the monitor (349) for switching between the sets of electrodes (143s) and/or circuits (267) of the sensing unit (571) to the monitor

13. The equipment for increasing of the number of leads for the ECG devices according to claim 1, characterized in that
the connecting field (711) is adapted for connecting to the monitor (349) of the electrodes (143s) comprised in the sensing unit (571),
which are divided into
a primary set of electrodes (143) and/or of circuits (267),
which are connected to the monitor (349) primarily, and
a secondary set of electrodes (143s) and/or of circuits (267), which are connected to the monitor (349) secondarily,
and preferably a tertiary set, and further sets of electrodes (143) and circuits (267), which are connected to the monitor successively (349),
wherein, the sets have an optional number of electrodes (143) and/or circuits (267) for sensing of the cardiac signals for processing by the monitor (349).

14. The equipment for increasing of the number of leads for the ECO devices according to claim 1, characterized in that
the connecting elements (486) are adapted for switching over of the monitor (349) from sensing of the primary electrodes (143p), which are located on the monitor (349), or on the cooperating unit (121), in which the monitor (349) is located, to sensing from electrodes (143s), or circuits (267) in the sensing unit (571),
wherein, preferably, the connecting elements (486) are formed by spring contacts (814) and they are connected to the electrodes (143p).
and/or, preferably, the connecting elements (486) are formed by a connector (480), which is connected to the monitor (349) and the connecting elements (486) are connected by conductors to the electrodes (143) in the sensing unit (571), preferably through a connecting field (711).

15. The equipment for increasing of the number of leads for the ECG devices according to claim 12, 13, characterized in that
the connecting field (711) is adapted for switching over of signals to the input of the monitor (349) from the primary set of electrodes (143p) to signals from the secondary set of electrodes (143s), and/or of circuits (267), and preferably to signals from further sets of electrodes (143s), which are comprised in the sensing unit (571) and
the connecting field (711) is connected between the monitor (349) and the sensing unit (571) and it is controlled by a controlling element (795) of the connecting field (711).

16. The equipment for increasing of the number of leads for the ECO devices according to claim 15, characterized in that
the connecting field (711) is adapted for initial interconnection with the primary set of a selected number of the primary electrodes (143) and/or circuits (267) to the monitor (349) for sensing of the analog cardiac signals for the initial processing to digital data to at least one ECG lead,
and after the initial processing of the cardiac signals by the monitor (349), and after securing of the digital data by saving it in the memory (961),
and/or by transmitting the data with a communication module (22) to the cooperating units (121),
preferably, the connecting field (711) is adapted for the second interconnection,
and in this case of a secondary set of the secondary electrodes (143s) and/or of the circuit (259) to the monitor (349)
for the second sensing of cardiac signals for the second processing to digital data by the monitor (349) to at least one ECG lead,
and after the secondary processing of the cardiac signals by the monitor (349) to ECG leads and after securing of the digital data,
preferably, the connecting field (711) is adapted for the next interconnection of further sets of electrodes (143) and/or circuits (259) to the monitor (349).

17. The equipment for increasing of the number of leads for the ECG devices according to claim 1, characterized in that
the switching elements (367), preferably formed by a switch, are adapted for switching over of the monitor (349) in the sensing unit (571) from the primary electrodes (143p) to the secondary electrodes (143s) by disconnection of the primary electrodes (143p) and by connecting of the monitor (349) in the sensing unit (571) to the secondary electrodes (143s).

18. The equipment for increasing of the number of leads for the ECG devices according to claim 1, characterized in that
the connecting field (711) is adapted for switching over of the monitor (349) from the primary electrodes (143p) to the secondary electrodes (143s) of the sensing unit (571) and preferably also for switching between the sets of electrodes (143) of the sensing unit (571) to the monitor (349),
wherein, the connecting field (711) is connected between the monitor and the sensing unit (571)
and it is controlled by a controlling element (795), which is located so as to allow local controlling on the sensing unit (711) or near the sensing unit (711),
which, for long-distance controlling, is connected by a cable, or which is connected wirelessly for remote control,
or which is connected via a local data network, or via Internet, to control with a long-distance control (800).

19. The equipment for increasing of the number of leads for the ECG devices according to claim 1, characterized in that
it comprises a memory (961), which is adapted for successive storage of digital data, which is processed successively by monitor (349) from the analog cardiac signals, which are sensed successively, preferably from the primary, secondary, and preferably further sets of the electrodes (143s) for the subsequent transmission via the communication circuits (368) of the digital data, successively stored in the memory (961),
for its evaluation and/or for successive or complete displaying of the ECG curves into at least one cooperating unit (121).

20. The equipment for increasing of the number of leads for the ECG devices according to claim 1, characterized in that
the communication circuits (368) are adapted for live transmission, or for transmission from the memory (961) of the digital data processed by monitor (349) from the memory (961), or for transmission of the digital data processed by monitor (349) successively and stored in the memory (961), to at least one cooperating unit (121), which is adapted for live displaying of a ECG curve, which corresponds to the live transmitted digital data, which are processed by the monitor (349),
or the cooperating unit (121) is adapted for successive or actual displaying of an ECG curve corresponding to the transmitted digital data, which are transmitted successively or simultaneously via the communication circuits (368).

21. The equipment for increasing of the number of leads for the ECG devices according to claim 1, characterized in that the independently located monitor (349) is connected to at least two primary electrodes (143p), which are located on the monitor (349),
or the monitor (349) is located on/in a cooperating unit (121), which is connected to at least 2 primary electrodes (143p), which are located on the cooperating unit (121),
wherein, switching over from sensing from the primary electrodes (143p), which are located on the (349) or on the cooperating unit (121), to sensing from the secondary electrodes (143s) of the sensing unit (571) is carried out by connecting elements (486) directly into the monitor (349) or via the electrodes (143p) located on the monitor (349) or on the cooperating unit (121).

22. The equipment for increasing of the number of leads for the ECG devices according to claim 1, characterized in that
the connecting elements (486) for interconnection by a connecting field (771) directly to the monitor (349) are formed by the connector (480) and for the interconnection via the electrodes (143) the connecting elements (486) are formed by flexible contacts (189) on electrodes (143), which are located on the monitor (349) or on the cooperating unit (121), in which the monitor (349) is located.

23. The equipment for increasing of the number of leads for the ECG devices according to claim 22, characterized in that
the monitor (349) is adapted for connecting of the primary electrodes (143p) for sensing of cardiac signals for at least one lead, and further it is adapted for switching over to the sensing unit (571), which is adapted for successive sensing from the sets of secondary electrodes (143s) for processing by the monitor (349) for displaying of more leads, than for which the primary electrodes (143p) are adapted, and the monitor is able to process simultaneously.

24. The equipment for increasing of the number of leads for the ECG devices according to claim 1, characterized in that
the monitor (349) is connected to the primary electrodes (143p), which are located externally with regard to the monitor (349), or on the monitor (349) or on the cooperating unit (121), on which/or in which the monitor (349) is located, wherein, the monitor (349) is adapted for switching over of the monitor (349) to electrodes (143) of the sensing unit (571) via the connecting field (711).

25. The equipment for increasing of the number of leads for the ECG devices according to claim 1, characterized in that
the connecting field (711), which is located within the monitor, or within the cooperating unit (121), in which, or on which the monitor (349) is located, is adapted for switching over of the monitor (349) for sensing of the cardiac signals from the primary electrodes (143) to sensing from electrodes (143s) of the sensing unit (571), preferably via the connecting elements (486).

26. The equipment for increasing of the number of leads for the ECG devices according to claim 1, characterized in that
the monitor (349) is located in the cooperating unit (121), which is formed preferably by a smart watch (416) used for displaying of an ECG from the digital data, which are processed by the monitor (349) from the cardiac signals sensed by primary electrodes (143p), which are located on the smart watch (416), which is adapted for switching over of the monitor (349) from sensing with the primary electrodes (143p) to sensing with the secondary electrodes (143s), which are located in the sensing unit (571), preferably via the connecting field (711), with connecting elements (486), which are connected to the primary electrodes (143p).

27. The equipment for increasing of the number of leads for the ECG devices according to claim 26, characterized in that
the monitor (349), which is located in the cooperating unit (121), which unit is formed by smart watch (416), is for sensing of cardiac signals interconnected to two primary electrodes (143p), which are located on the smart watch (416), which electrodes are formed by the electrodes (143′) for a wrist, used for sensing from a wrist, and an electrode (143″) for sensing from a finger,
and the monitor (349) is adapted for switching over via the connecting element (486),
and preferably, the connecting field (711) for sensing from the sensing unit (571) for successive interconnection of the electrode sets (143s), which are located in the sensing unit (571) for an increased number of the ECG leads, which the monitor (349) can process successively from the cardiac signals from the connected secondary electrodes (143s) of the sensing unit (571), in comparison to the original one ECG lead, which the monitor is adapted to process from two primary electrodes (143p).
and preferably, the connecting field (711) for sensing from the sensing unit (571) for successive interconnection of the electrode sets (143s),
which are located in the sensing unit (571) for an increased number of the ECG leads, which the monitor (349) can process successively from the cardiac signals from the connected secondary electrodes (143s) of the sensing unit (571),
in comparison to the original one ECG lead, which the monitor is adapted to process from two primary electrodes (143p).

28. The equipment for increasing of the number of leads for the ECG devices according to claim 27, characterized in that
during the period of sensing with the sensing unit (571), the base (259) is located under the smart watch (416) for fixing of the base by pushing it with the smart watch (416) by the action of the force exerted by the watch strap (394) and for isolating of the electrodes (143″) from the wrist.

29. The equipment for increasing of the number of leads for the ECG devices according to claim 28, characterized in that
the connecting element (486) for connecting of the base (259) to the electrode (143′) for a wrist is formed by a contact (189) of the base, and to the electrode (143″) for a finger it is formed by a contact (258) of the base, wherein, the connecting elements (486) are located on the base (259), which base is located for connecting of the electrodes under the smart watch (416) temporarily.

30. The equipment for increasing of the number of leads for the ECG devices according to claim 29, characterized in that
for temporary fixing, the base (259) is inserted under the smart watch (416), where it isolates the electrode (143″) for a wrist from a wrist and connects it to the sensing unit (711) by a connecting element (486), preferably through a connecting field (711), together with an electrode (143′) for a finger with another connecting element (486) for sensing from the sensing unit (571), and for sensing from the electrodes (143″, 143′) located on the smart watch (416), the base (259) is removed.

31. The equipment for increasing of the number of leads for the ECG devices according to claim 28, 29, characterized in that
the other contact (258) of the base is located on the sliding part of the base, which, for the switching over to the electrode (143″), moves to the smart watch (416), whereby, preferably, it is connected to the contact (189) of the base, which contact connects the other contact (258) of the base by interconnection (395) to the sensing unit (571), preferably via the connecting field (711), or it is firmly connected with the base, and the smart watch (416) slides into the base, preferably from above.

32. The equipment for increasing of the number of leads for the ECG devices according to claim 1, characterized in that
the cardiac signals, sensed by electrodes (143s), are processed by the monitor (349) to digital signals for displaying of the ECG leads, at least of one of the leads from: I, II, V1 to V6, V1R to V6R, V7 to V9, wherein, optionally, four leads: III, aVR, aVL, and aVF are calculated.

33. The equipment for increasing of the number of leads for the ECG devices according to claim 1, characterized in that
the monitor (349) for attaching, which is formed by the monitor (770),
with electrodes (143) formed by the primary electrodes (143p), which are formed by the electrodes (771) located on the surface of the monitor (770),
is adapted for sensing from primary electrodes (143p) by attaching it to fingers or to chest and is adapted for switching over to sensing from secondary electrodes (143s) of the sensing unit (571),
preferably by interconnecting it via an external connecting field (711),
with connecting elements (486), which are formed by a connector (480) and a cable (640), or it is connected with the connecting elements (486), which are formed by spring contacts (714) which are located preferably on the holder (774),
which is connected to the primary electrodes (143p),
and the spring contacts (714) are connected to electrodes (143s) of the sensing unit (571) with a cable (640), preferably via the external connecting field (711), or they are located on the holder (774),
and the connecting field (711) is interconnected to the secondary electrodes (143s) of the primary sensing unit (571),
from which the sets of electrodes (143s) for successive sensing of cardiac signals are connected, preferably successively, for processing of the cardiac signals by monitor (349) to more ECG leads than what the monitor (349) is able to process from the primary electrodes (143p) at one time.

34. The equipment for increasing of the number of leads for the ECG devices according to claim 1, characterized in that
the sensing unit (571) comprises an electrode (143) formed by relocateable electrode (143m) serving for successive sensing of the chest leads,
and preferably, the sensing unit (571) is formed by the sensing unit (571) with electrodes to be applied to the body for one-time or short-term monitoring of up to 21-lead ECG, or by a base (765) in the form of a belt for long-term or continuous monitoring.

35. The equipment for increasing of the number of leads for the ECG devices according to claim 1, characterized in that
the sensing unit (571) is formed with at least one of the following: the base (705), the base (707) for the chest leads, the multi-electrode base (630), the base (445) of the monitor for multi-lead ECO sensing with a small-plate provided with two electrodes, or with the plate (434) of leg electrodes, provided with three electrodes, the abdominal three-channel base (440) of the monitor with the electrode (450) of the second small-plate of two electrodes, the multi-channel base (468) with the chest small-plate (457) of two electrodes, or the plate (465) of the chest electrodes, base (420) of the universal monitor, base (620) for RL, base (622) for RL and twice V, elongated base (624), base (667) with the reference, complete base (712), base (789) in the form of a belt, base (720), base (705) for LL, RL with the glued electrode (1074).

36. The equipment for increasing of the number of leads for the ECG devices according to claim 1, characterized in that
in case of the monitor (349), which is located in the unit (369), provided with external electrodes, which are formed by external primary electrodes (143p), which are connected by a cable (640) via the connecting elements (486), preferably formed by the connector (480),
the connection to the sensing unit (571), which is formed preferably by a sensing unit in the form of a belt, preferably by the multi-electrode base (630),
is carried out by disconnecting of the primary electrodes (143p) by disconnecting of the connecting element (486),
which element are the primary electrodes (143p), which are connected to the unit (369) and by connecting of another connecting element (486),
preferably of the connector (480) provided with connected sensing unit (571),
preferably via the connecting field (711),
for successive sensing of cardiac signals from the secondary electrodes (143s), which are successively changed over through the connecting field (711)
for processing by the monitor (349) to more ECO leads, than what the monitor (349) is able to process from the primary electrodes (143p).

37. The equipment for increasing of the number of leads for the ECG devices according to claim 1, characterized in that
the unit (369) with external electrodes is formed by one of the following units: the ECG device (19), the Holter device (370), the monitor (393) at a hospital bed.

38. The equipment for increasing of the number of leads for the ECG devices according to claim, characterized in that
the connecting elements (486) are adapted for switching over between the sensing from the primary electrodes (143p) and sensing from the secondary electrodes (143s) of the sensing unit (571) by connecting the sensing units (571) to the monitor (349) instead of the electrodes (143p) through the connecting elements (486), which are formed preferably by the connector (480) or by the spring contacts (814).

39. The equipment for increasing of the number of leads for the ECG devices according to claim 1, characterized in that
the monitor is adapted for relocation from one sensing unit (571), which senses preferably cardiac signals with the primary electrodes (143p), to the second sensing unit (571),
and preferably to another sensing unit (571) with secondary electrodes (143s) for sensing of cardiac signals for more leads than what the primary electrodes (143p) are able to sense.

40. The equipment for increasing of the number of leads for the ECG devices according to claim 1, characterized in that
the monitor is adapted for relocation from the sensing unit (571) with primary electrodes (143p), which unit allows continuous sensing, formed by chest belt (749), preferably provided with united electrodes (471) for relocation to the sensing unit (571), preferably formed by base (667) with the reference or base (705) for LL, RL, which comprises at least one relocateable electrode (143m) for sensing of more chest leads than what allow the electrodes (143p),
or relocation to the base (571) formed by the chest belt (749), preferably formed by the multi-electrode base (630) to allow sensing of cardiac signals for their processing by the monitor (349) to more ECO leads, than how many allows the sensing unit (571) with primary electrodes (143p), which is formed by a chest belt (749) with electrodes (471).

41. The equipment for increasing of the number of leads for the ECG devices according to claim 1, characterized in that
the monitor (349), which is independent or located in the cooperating unit (121) for one-time or short-term sensing with the primary electrodes (143p), which are located on the monitor (349) or on the cooperating unit (121) or located externally, and connected by a cable (640), is adapted for switching over from primary electrodes (143p) to the secondary electrodes (143s) on the sensing unit (571), preferably in the form of a belt, which (what) allows the long term or continuous sensing.
